(19) United States
(12) Patent Application Publication
(10) Pub. No.: US 2016/0102074 A1
(43) Pub. Date: Apr. 14, 2016

(54) SUBSTITUTED AMIDE COMPOUNDS

(71) Applicant: PFIZER INC., New York, NY (US)

(72) Inventors: Etzer Darout, Sharon, MA (US); Kim F. McClure, Mystic, CT (US); David Piotrowski, Waterford, CT (US); Brian Raymer, Holliston, MA (US)

(73) Assignee: PFIZER INC., New York, NY (US)

(21) Appl. No.: 14/868,933

(22) Filed: Sep. 29, 2015

Related U.S. Application Data
(60) Provisional application No. 62/211,082, filed on Aug. 28, 2015.
(60) Provisional application No. 62/171,514, filed on Jun. 5, 2015.
(60) Provisional application No. 62/061,275, filed on Oct. 8, 2014.

(51) Int. Cl. C07D 401/14; A61K 45/06; A61K 31/4545

(52) U.S. Cl. CPC C07D 401/14; A61K 31/4545; A61K 45/06

(57) ABSTRACT

The present invention is directed at substituted amide compounds, pharmaceutical compositions containing such compounds and the use of such compounds to reduce plasma lipid levels, such as LDL-cholesterol and triglycerides and accordingly to treat diseases which are exacerbated by high levels of LDL-cholesterol and triglycerides, such as atherosclerosis and cardiovascular diseases, in mammals, including humans.

### BACKGROUND OF INVENTION

The present invention relates to substituted amide compounds, pharmaceutical compositions containing such compounds and the use of such compounds to treat cardiovascular disease including atherosclerosis, hyperlipidemia, hypercholesterolemia, and hypertriglyceridemia in mammals, including humans.

Atherosclerosis, a disease of the arteries, is recognized to be the leading cause of death in the United States and Western Europe. The pathological sequence leading to atherosclerosis and occlusive heart disease is well known. The earliest stage in this sequence is the formation of “fatty streaks” in the carotid, coronary and cerebral arteries and in the aorta. These lesions are yellow in color due to the presence of lipid deposits found principally within smooth-muscle cells and in macrophages of the intima layer of the arteries and aorta. Further, it is postulated that most of the cholesterol found within the fatty streaks, in turn, gives rise to development of the “fibrous plaque,” which consists of accumulated intimal smooth muscle cells laden with lipid and surrounded by extra-cellular lipid, collagen, elastin and proteoglycans. These cells plus matrix form a fibrous cap that covers a deeper deposit of cell debris and more extracellular lipid. The lipid is primarily free and esterified cholesterol. The fibrous plaque forms slowly, and is likely in time to become calcified and necrotic, advancing to the “complicated lesion,” which accounts for the arterial occlusion and tendency toward mural thrombosis and arterial muscle spasm that characterize advanced atherosclerosis.

Epidemiological evidence has firmly established hyperlipidemia as a primary risk factor in causing cardiovascular disease (CVD) due to atherosclerosis. In recent years, leaders of the medical profession have placed renewed emphasis on lowering plasma cholesterol levels, and low density lipoprotein cholesterol in particular, as an essential step in prevention of CVD. The upper limits of “normal” are now known to be significantly lower than heretofore appreciated. As a result, large segments of Western populations are now realized to be at particularly high risk. Additional independent risk factors include glucose intolerance, left ventricular hypertrophy, hypertension, and being of the male sex. Cardiovascular disease is especially prevalent among diabetic subjects, at least in part because of the existence of multiple independent risk factors in this population. Successful treatment of hyperlipidemia in the general population, and in diabetic subjects in particular, is therefore of exceptional medical importance.

While there are a variety of anti-atherosclerosis compounds, cardiovascular disesease is still a leading cause of death and accordingly, there is a continuing need and a continuing search in this field of art for alternative therapies.

### SUMMARY OF THE INVENTION

The present invention is directed to compounds of Formula I

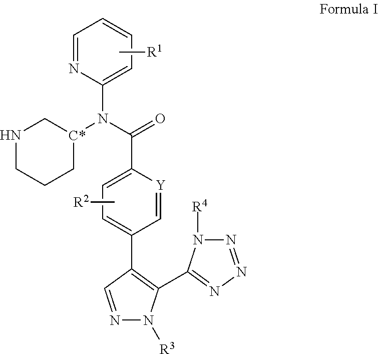

or a pharmaceutically acceptable salt thereof wherein
R¹ is optionally chloro or (C₁-C₂)alkyl;
Y is independently either N or C(H);
R² is H or fluoro;
R³ is H or (C₁-C₂)alkyl; and
R⁴ is (C₁-C₂)alkoxycarbonyloxy(C₁-C₂)alkyl;
with the proviso that diastereomeric mixture ethyl 1-{5-[1-methyl-4-(4-{(3-methylpyridin-2-yl)[(3R)-piperidin-3-yl]carbamoyl}phenyl)-1H-pyrazol-5-yl]-1H-tetrazol-1-yl}ethyl carbonate is not included. This does not mean that the individual diastereomers are not included.

The present invention is directed to compounds of Formula II

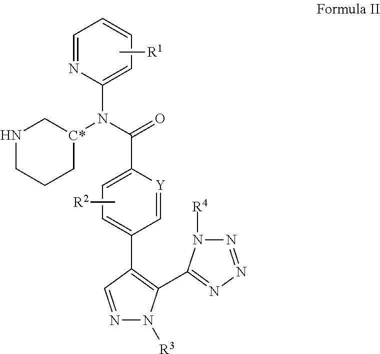

or a pharmaceutically acceptable salt thereof wherein
R¹ is optionally chloro or (C₁-C₂)alkyl;
Y is independently either N or C(H);
R² is H or fluoro;
R³ is H or (C₁-C₂)alkyl; and

### R⁴ is H;

with the proviso that N-(3-methylpyridin-2-yl)-4-[1-methyl-5-(2H-tetrazol-5-yl)-1H-pyrazol-4-yl]-N-[(3R)-piperidin-3-yl]benzamide and N-(3-chloropyridin-2-yl)-4-[1-methyl-5-(2H-tetrazol-5-yl)-1H-pyrazol-4-yl]-N-[(3R)-piperidin-3-yl]benzamide are not included.

The present application is also directed to methods for treating dyslipidemia, hypercholesterolemia (including heterozygous and homozygous familial hypercholesterolemia), hypertriglyceridemia, hyperlipidemia, hypoalphalipoproteinemia, metabolic syndrome, diabetic complications, atherosclerosis, stroke, vascular dimensia, chronic kidney disease, coronary heart disease, coronary artery disease, retinopathy, inflammation, thrombosis, peripheral vascular disease or congestive heart failure in a mammal by administering to a mammal in need of such treatment a therapeutically effective amount of a compound of Formula I or II or a pharmaceutically acceptable salt of said compound.

The present application also is directed to pharmaceutical compositions which comprise a therapeutically effective amount of a compound of Formula I or II, or a pharmaceutically acceptable salt of said compound and a pharmaceutically acceptable carrier, vehicle or diluent.

In addition, the present application is directed to pharmaceutical combination compositions comprising: a therapeutically effective amount of a composition comprising

a first compound, said first compound being a compound of Formula I or II or a pharmaceutically acceptable salt of said compound;

a second compound, said second compound being a lipid modulating agent; and

a pharmaceutically acceptable carrier, vehicle or diluent.

Examples of lipid modulating agents include a lipase inhibitor, an HMG-CoA reductase inhibitor, an HMG-CoA synthase inhibitor, an HMG-CoA reductase gene expression inhibitor, an HMG-CoA synthase gene expression inhibitor, an MTP/Apo B secretion inhibitor, a CETP inhibitor, a bile acid absorption inhibitor, a cholesterol absorption inhibitor, a cholesterol synthesis inhibitor, a squalene synthetase inhibitor, a squalene epoxidase inhibitor, a squalene cyclase inhibitor, a combined squalene epoxidase/squalene cyclase inhibitor, a fibrate, niacin, a combination of niacin and lovastatin, an ion-exchange resin, an antioxidant, an ACAT inhibitor and a bile acid sequestrant.

It is to be understood that both the foregoing general description and the following detailed description are exemplary and explanatory only and are not restrictive of the invention, as claimed.

### BRIEF DESCRIPTION OF THE FIGURES

FIG. 1 is an X-ray crystal structure (ORTEP drawing) of Preparation 14a.

FIG. 2 is an X-ray crystal structure (ORTEP drawing) of Preparation 15c.

FIG. 3 is a characteristic X-ray powder diffraction pattern showing a crystalline form of Example 5a (Vertical Axis: Intensity (CPS); Horizontal Axis: Two theta (degrees)).

FIG. 4 is a characteristic X-ray powder diffraction pattern showing a crystalline form of Example 6 (Vertical Axis: Intensity (CPS); Horizontal Axis: Two theta (degrees)).

FIG. 5 is a characteristic X-ray powder diffraction pattern showing a crystalline form of Example 7 (Vertical Axis: Intensity (CPS); Horizontal Axis: Two theta (degrees)).

### DETAILED DESCRIPTION OF THE INVENTION

The present invention may be understood more readily by reference to the following detailed description of exemplary embodiments of the invention and the examples included therein.

Before the present compounds, compositions and methods are disclosed and described, it is to be understood that this invention is not limited to specific synthetic methods of making the compounds that may of course vary. It is also to be understood that the terminology used herein is for the purpose of describing particular embodiments only and is not intended to be limiting.

A preferred group of compounds, designated the A Group, contains those compounds having the Formula I as shown above wherein the piperidinyl C* is the R configuration and R⁴ is ethoxycarbonyloxyethyl.

A group of compounds which is preferred among the A Group of compounds designated the B Group, contains those compounds wherein Y is N.

A group of compounds which is preferred among the B Group of compounds designated the C Group, contains those compounds wherein R¹ is chloro or methyl; R² is H or fluoro; and R³ is H or methyl.

A group of compounds which is preferred among the A Group of compounds designated the D Group, contains those compounds wherein Y is C(H).

A group of compounds which is preferred among the D Group of compounds designated the E Group, contains those compounds wherein R¹ is chloro or methyl; R² is H or fluoro; and R³ is H or methyl.

A preferred group of compounds, designated the F Group, contains those compounds having the Formula II as shown above wherein the piperidinyl C* is the R configuration.

A group of compounds which is preferred among the F Group of compounds designated the G Group, contains those compounds wherein Y is C(H).

A group of compounds which is preferred among the G Group of compounds designated the H Group, contains those compounds wherein R¹ is chloro or methyl; R² is H or fluoro; and R³ is H or methyl.

A group of compounds which is preferred among the F Group of compounds designated the I Group, contains those compounds wherein Y is N.

A group of compounds which is preferred among the I Group of compounds designated the J Group, contains those compounds wherein R¹ is chloro or methyl; R² is H or fluoro; and R³ is H or methyl.

A preferred compound is ethyl (S)-1-{5-[1-methyl-4-(4-{(3-methylpyridin-2-yl)[(3R)-piperidin-3-yl]carbamoyl}phenyl)-1H-pyrazol-5-yl]-1H-tetrazol-1-yl}ethyl carbonate or a pharmaceutically acceptable salt thereof.

A preferred compound is

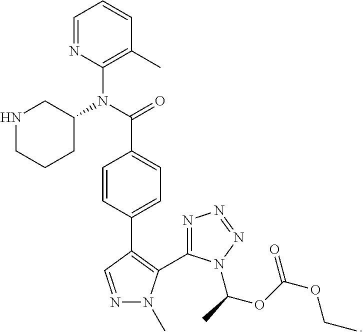 [SMILES: CCOC(=O)O[C@@H](C)n1nnnc1-c1c(-c2ccc(C(=O)N(c3ncccc3C)[C@@H]3CCCNC3)cc2)cnn1C]

A preferred compound is ethyl (R)-1-{5-[1-methyl-4-(4-{(3-methylpyridin-2-yl)[(3R)-piperidin-3-yl]carbamoyl}phenyl)-1H-pyrazol-5-yl]-1H-tetrazol-1-yl}ethyl carbonate or a pharmaceutically acceptable salt thereof.

A preferred compound is

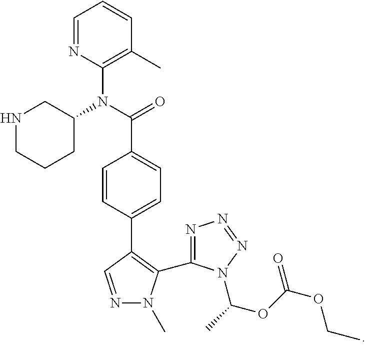 [SMILES: CCOC(=O)O[C@H](C)n1nnnc1-c1c(-c2ccc(C(=O)N(c3ncccc3C)[C@@H]3CCCNC3)cc2)cnn1C]

A preferred compound is ethyl (S)-1-{5-[1-methyl-4-(4-{(3-chloropyridin-2-yl)[(3R)-piperidin-3-yl]carbamoyl}phenyl)-1H-pyrazol-5-yl]-1H-tetrazol-1-yl}ethyl carbonate or a pharmaceutically acceptable salt thereof.

A preferred compound is

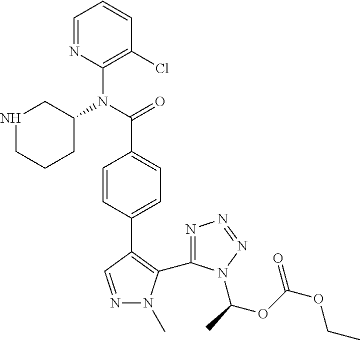 [SMILES: CCOC(=O)O[C@@H](C)n1nnnc1-c1c(-c2ccc(C(=O)N(c3ncccc3Cl)[C@@H]3CCCNC3)cc2)cnn1C]

A preferred compound is ethyl (S)-1-{5-[4-(4-{(3-chloropyridin-2-yl)[(3R)-piperidin-3-yl]carbamoyl}-2-fluorophenyl)-1-methyl-1H-pyrazol-5-yl]-1H-tetrazol-1-yl}ethyl carbonate or a pharmaceutically acceptable salt thereof.

A preferred compound is

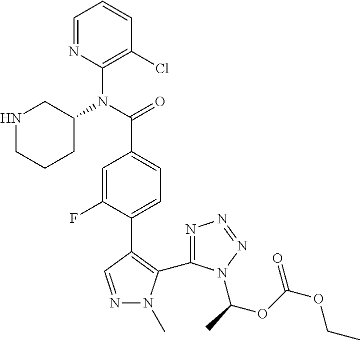 [SMILES: CCOC(=O)O[C@@H](C)n1nnnc1-c1c(-c2ccc(C(=O)N(c3ncccc3Cl)[C@@H]3CCCNC3)cc2F)cnn1C]

A preferred compound is ethyl (S)-1-{5-[4-(4-{(3-methylpyridin-2-yl)[(3R)-piperidin-3-yl]carbamoyl}-2-fluorophenyl)-1-methyl-1H-pyrazol-5-yl]-1H-tetrazol-1-yl}ethyl carbonate or a pharmaceutically acceptable salt thereof.

A preferred compound is

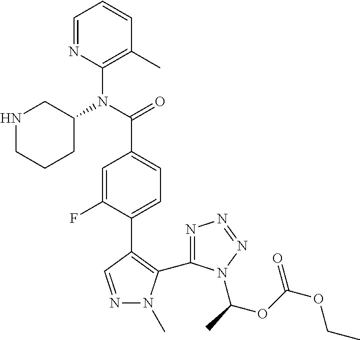 [SMILES: CCOC(=O)O[C@@H](C)n1nnnc1-c1c(-c2ccc(C(=O)N(c3ncccc3C)[C@@H]3CCCNC3)cc2F)cnn1C]

A preferred compound is ethyl (S)-1-{5-[1-methyl-4-(6-{(3-methylpyridin-2-yl)[(3R)-piperidin-3-yl]carbamoyl}pyridin-3-yl)-1H-pyrazol-5-yl]-1H-tetrazol-1-yl}ethyl carbonate or a pharmaceutically acceptable salt thereof.

A preferred compound is

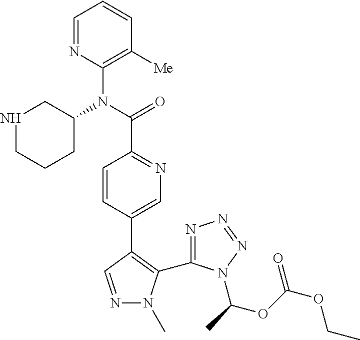 [SMILES: CCOC(=O)O[C@@H](C)n1nnnc1-c1c(-c2ccc(C(=O)N(c3ncccc3C)[C@@H]3CCCNC3)nc2)cnn1C]

A preferred is ethyl (S)-1-{5-[4-(6-{(3-chloropyridin-2-yl)[(3R)-piperidin-3-yl]carbamoyl}pyridin-3-yl)-1-methyl-1H-pyrazol-5-yl]-1H-tetrazol-1-yl}ethyl carbonate or a pharmaceutically acceptable salt thereof.

A preferred compound is

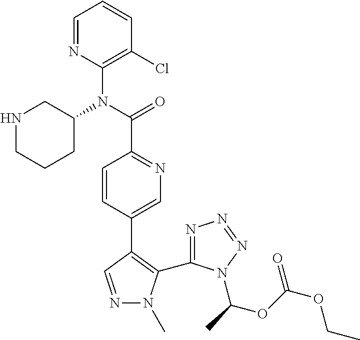 [SMILES: CCOC(=O)O[C@@H](C)n1nnnc1-c1c(-c2ccc(C(=O)N(c3ncccc3Cl)[C@@H]3CCCNC3)nc2)cnn1C]

A preferred compound is N-(3-methylpyridin-2-yl)-5-[1-methyl-5-(2H-tetrazol-5-yl)-1H-pyrazol-4-yl]-N-[(3R)-piperidin-3-yl]pyridine-2-carboxamide or a pharmaceutically acceptable salt thereof

A preferred compound is

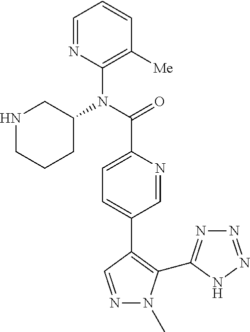 [SMILES: Cc1cccnc1N(C(=O)c1ccc(-c2cnn(C)c2-c2nnn[nH]2)cn1)[C@@H]1CCCNC1]

A preferred compound is N-(3-chloropyridin-2-yl)-5-[1-methyl-5-(2H-tetrazol-5-yl)-1H-pyrazol-4-yl]-N-[(3R)-piperidin-3-yl]pyridine-2-carboxamide or a pharmaceutically acceptable salt thereof.

A preferred compound is

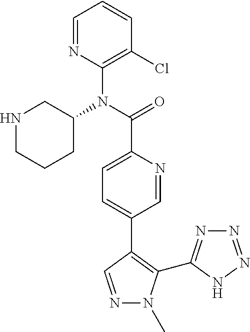 [SMILES: Cn1ncc(-c2ccc(C(=O)N(c3ncccc3Cl)[C@@H]3CCCNC3)nc2)c1-c1nnn[nH]1]

A preferred compound is N-(3-chloropyridin-2-yl)-3-fluoro-4-[1-methyl-5-(2H-tetrazol-5-yl)-1H-pyrazol-4-yl]-N-[(3R)-piperidin-3-yl]benzamide or a pharmaceutically acceptable salt thereof.

A preferred compound is

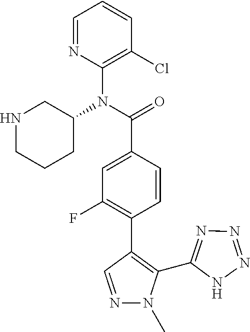 [SMILES: Cn1ncc(-c2ccc(C(=O)N(c3ncccc3Cl)[C@@H]3CCCNC3)cc2F)c1-c1nnn[nH]1]

A preferred compound is N-(3-methylpyridin-2-yl)-3-fluoro-4-[1-methyl-5-(2H-tetrazol-5-yl)-1H-pyrazol-4-yl]-N-[(3R)-piperidin-3-yl]benzamide or a pharmaceutically acceptable salt thereof.

A preferred compound is

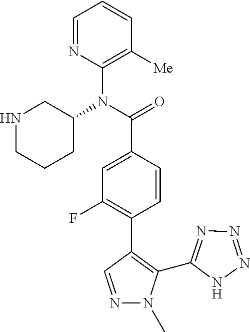 [SMILES: Cc1cccnc1N(C(=O)c1ccc(-c2cnn(C)c2-c2nnn[nH]2)c(F)c1)[C@@H]1CCCNC1]

A preferred compound is ethyl (R)-1-{5-[4-(4-{(3-chloropyridin-2-yl)[(3R)-piperidin-3-yl]carbamoyl}-2-fluorophenyl)-1-methyl-1H-pyrazol-5-yl]-1H-tetrazol-1-yl}ethyl carbonate or a pharmaceutically acceptable salt thereof.

A preferred compound is

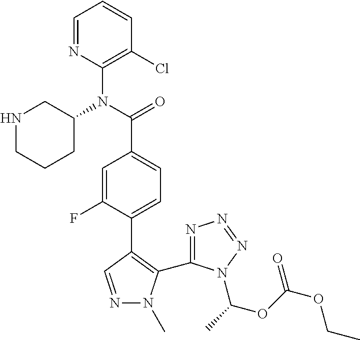 [SMILES: CCOC(=O)O[C@H](C)n1nnnc1-c1c(-c2ccc(C(=O)N(c3ncccc3Cl)[C@@H]3CCCNC3)cc2F)cnn1C]

A preferred compound is ethyl (R)-1-{5-[1-methyl-4-(4-{(3-chloropyridin-2-yl)[(3R)-piperidin-3-yl]carbamoyl}phenyl)-1H-pyrazol-5-yl]-1H-tetrazol-1-yl}ethyl carbonate or a pharmaceutically acceptable salt thereof.

A preferred compound is:

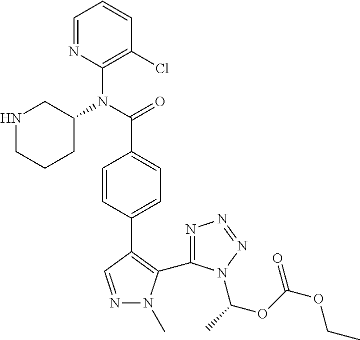 [SMILES: CCOC(=O)O[C@H](C)n1nnnc1-c1c(-c2ccc(C(=O)N(c3ncccc3Cl)[C@@H]3CCCNC3)cc2)cnn1C]

A preferred group of compounds, designated the P Group, contains the following compounds ethyl (S)-1-{5-[1-methyl-4-(4-{(3-methylpyridin-2-yl)[(3R)-piperidin-3-yl]carbamoyl}phenyl)-1H-pyrazol-5-yl]-1H-tetrazol-1-yl}ethyl carbonate;
ethyl (R)-1-{5-[1-methyl-4-(4-{(3-methylpyridin-2-yl)[(3R)-piperidin-3-yl]carbamoyl}phenyl)-1H-pyrazol-5-yl]-1H-tetrazol-1-yl}ethyl carbonate;
ethyl (S)-1-{5-[1-methyl-4-(4-{(3-chloropyridin-2-yl)[(3R)-piperidin-3-yl]carbamoyl}phenyl)-1H-pyrazol-5-yl]-1H-tetrazol-1-yl}ethyl carbonate;
ethyl (S)-1-{5-[4-(4-{(3-chloropyridin-2-yl)[(3R)-piperidin-3-yl]carbamoyl}-2-fluorophenyl)-1-methyl-1H-pyrazol-5-yl]-1H-tetrazol-1-yl}ethyl carbonate;
ethyl (S)-1-{5-[4-(4-{(3-methylpyridin-2-yl)[(3R)-piperidin-3-yl]carbamoyl}-2-fluorophenyl)-1-methyl-1H-pyrazol-5-yl]-1H-tetrazol-1-yl}ethyl carbonate;
ethyl (S)-1-{5-[1-methyl-4-(6-{(3-methylpyridin-2-yl)[(3R)-piperidin-3-yl]carbamoyl}pyridin-3-yl)-1H-pyrazol-5-yl]-1H-tetrazol-1-yl}ethyl carbonate; or
ethyl (S)-1-{5-[4-(6-{(3-chloropyridin-2-yl)[(3R)-piperidin-3-yl]carbamoyl}pyridin-3-yl)-1-methyl-1H-pyrazol-5-yl]-1H-tetrazol-1-yl}ethyl carbonate or a pharmaceutically acceptable salt of said each of said compounds.

A preferred group of compounds, designated the Q Group, contains the following compounds
N-(3-methylpyridin-2-yl)-5-[1-methyl-5-(2H-tetrazol-5-yl)-1H-pyrazol-4-yl]-N-[(3R)-piperidin-3-yl]pyridine-2-carboxamide;
N-(3-chloropyridin-2-yl)-5-[1-methyl-5-(2H-tetrazol-5-yl)-1H-pyrazol-4-yl]-N-[(3R)-piperidin-3-yl]pyridine-2-carboxamide;
N-(3-chloropyridin-2-yl)-3-fluoro-4-[1-methyl-5-(2H-tetrazol-5-yl)-1H-pyrazol-4-yl]-N-[(3R)-piperidin-3-yl]benzamide; or
N-(3-methylpyridin-2-yl)-3-fluoro-4-[1-methyl-5-(2H-tetrazol-5-yl)-1H-pyrazol-4-yl]-N-[(3R)-piperidin-3-yl]benzamide
   or a pharmaceutically acceptable salt of any of said compounds.

Another preferred group of compounds is each of the compounds in the P and Q groups taken individually.

It is also preferred that each of those compounds taken individually is a pharmaceutically acceptable salt, and especially preferred that each taken individually is an acid addition salt thereof. It is also especially preferred that the salt is the hydrochloride salt.

References to Compounds of Formula I or the like below are herein defined to also include Compounds of Formula II.

In one preferred embodiment of the pharmaceutical combination compositions, methods and kits of the present invention, the second compound is an HMG-CoA reductase inhibitor or a CETP inhibitor, such as rosuvastatin, rivastatin, pitavastatin, lovastatin, simvastatin, pravastatin, fluvastatin, atorvastatin or cerivastatin or a prodrug of said compound or a pharmaceutically acceptable salt of said compound or prodrug. It is especially preferred that the second compound is atorvastatin hemi-calcium.

Pharmaceutically acceptable salts of the compounds of Formula I include the acid addition and base salts thereof. Pharmaceutically acceptable salts of the compounds of Formula I formed with acids are preferred. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts.

Suitable base salts are formed from bases which form non-toxic salts. Examples include the aluminium, arginine, calcium, choline, diethylamine, glycine, lysine, magnesium, meglumine, olamine, potassium, sodium, trimethamine and zinc salts. Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, 2002).

The compounds of the invention may exist in both unsolvated and solvated forms. The term ‘solvate’ is used herein to describe a molecular complex comprising the compound of the invention and one or more pharmaceutically acceptable solvent molecules, for example, ethanol. Such solvent molecules are those commonly used in the pharmaceutical art, which are known to be innocuous to the recipient, e.g., water, ethanol, and the like. Other solvents may be used as intermediate solvates in the preparation of more desirable solvates, such as methanol, methyl t-butyl ether, ethyl acetate, methyl acetate, (S)-propylene glycol, (R)-propylene glycol, 1,4-butyne-diol, and the like. The term ‘hydrate’ is employed when said solvent is water. Pharmaceutically acceptable solvates include hydrates and other solvates wherein the solvent of crystallization may be isotopically substituted, e.g. D₂O, d₆-acetone, d₆-DMSO. The term “hydrate” refers to the complex where the solvent molecule is water. The solvates and/or hydrates preferably exist in crystalline form.

The compounds of the invention may also exist as complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components which may be in stoichiometric or non-stoichiometric amounts. The resulting complexes may be ionised, partially ionised, or non-ionised. For a review of such complexes, see J Pharm Sci, 64 (8), 1269-1288 by Haleblian (August 1975).

The compounds of the invention include compounds of Formula I as hereinbefore defined, polymorphs, and isomers thereof (including optical, geometric and tautomeric isomers including compounds exhibiting more than one type of isomerism, and mixtures of one or more thereof) and isotopically-labelled compounds of Formula I. Thus, the compounds of the present invention can exist in the form of various stereoisomers, R and S isomers, depending upon the presence of asymmetric carbon atoms. Herein, they may be referred to as the “R configuration” or “S configuration” or the like. The present invention encompasses both the individual isomers and mixtures thereof, including racemic and diastereomeric mixtures.

Compounds of Formula I containing an asymmetric carbon atom can exist as two or more stereoisomers. Alpha and Beta refer to the orientation of a substituent with reference to the plane of the ring. Beta is above the plane of the ring and Alpha is below the plane of the ring.

Where a compound of Formula I contains an alkenyl or alkenylene group or a cycloalkyl group, geometric cis/trans (or Z/E) isomers are possible. Thus, compounds of the invention exist as cis or trans configurations and as mixtures thereof. The term “cis” refers to the orientation of two substituents with reference to each other and the plane of the ring (either both “up” or both “down”). Analogously, the term “trans” refers to the orientation of two substituents with reference to each other and the plane of the ring (the substituents being on opposite sides of the ring).

Where the compound contains, for example, a keto or oxime group or an aromatic moiety, tautomeric isomerism (‘tautomerism’) can occur.

The present invention includes all pharmaceutically acceptable isotopically-labelled compounds of Formula I wherein one or more atoms are replaced by atoms having the same atomic number, but an atomic mass or mass number different from the atomic mass or mass number usually found in nature.

Examples of isotopes suitable for inclusion in the compounds of the invention include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulphur, such as ³⁵S.

Certain isotopically-labelled compounds of Formula (I), for example, those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection.

Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Tomography (PET) studies for examining substrate receptor occupancy.

References herein to “treat”, “treating”, “treatment” and the like include curative, palliative and prophylactic treatment.

As used herein, the expressions “reaction-inert solvent” and “inert solvent” refer to a solvent or a mixture thereof which does not interact with starting materials, reagents, intermediates or products in a manner which adversely affects the yield of the desired product.

By “pharmaceutically acceptable” is meant the carrier, vehicle, or diluent and/or salt must be compatible with the other ingredients of the formulation, and not deleterious to the recipient thereof.

The term “pharmaceutically effective amount”, as used herein, refers to an amount of the compound of Formula I (or a combination agent or a Formula I compound in combination with a combination agent) sufficient to treat, prevent onset of or delay or diminish the symptoms and physiological manifestations of the indications described herein.

The term “room temperature or ambient temperature” means a temperature between 18 to 25° C., “HPLC” refers to high pressure liquid chromatography, “MPLC” refers to medium pressure liquid chromatography, “TLC” refers to thin layer chromatography, “MS” refers to mass spectrum or mass spectroscopy or mass spectrometry, “NMR” refers to nuclear magnetic resonance spectroscopy, “DCM” refers to dichloromethane, “DMSO” refers to dimethyl sulfoxide, “DME” refers to dimethoxyethane, “EtOAc” refers to ethyl acetate, “MeOH” refers to methanol, “Ph” refers to the phenyl group, “Pr” refers to propyl, “trityl” refers to the triphenylmethyl group, “ACN” refers to acetonitrile, “DEAD” refers to diethylazodicarboxylate, and “DIAD” refers to diisopropylazodicarboxylate.

It is to be understood that if a carbocyclic or heterocyclic moiety may be bonded or otherwise attached to a designated substrate through differing ring atoms without denoting a specific point of attachment, then all possible points are intended, whether through a carbon atom or, for example, a trivalent nitrogen atom. For example, the term “pyridyl” means 2-, 3-, or 4-pyridyl, the term “thienyl” means 2-, or 3-thienyl, and so forth. In general the compounds of this invention can be made by processes which include processes analogous to those known in the chemical arts, particularly in light of the description contained herein.

The term “coronary artery disease”, as used herein, is selected, but not limited to, the group consisting of atherosclerotic plaque (e.g., prevention, regression, stablilization), vulnerable plaque (e.g., prevention, regression, stabilization), vulnerable plaque area (reduction), arterial calcification (e.g., calcific aortic stenosis), increased coronary artery calcium score, dysfunctional vascular reactivity, vasodilation disorders, coronary artery spasm, first myocardial infarction, myocardia re-infarction, ischemic cardiomyopathy, stent restenosis, PTCA restenosis, arterial restenosis, coronary bypass graft restenosis, vascular bypass restenosis, decreased exercise treadmill time, angina pectoris/chest pain, unstable angina pectoris, exertional dyspnea, decreased exercise capacity, ischemia (reduce time to), silent ischemia (reduce time to), increased severity and frequency of ischemic symptoms, reperfusion after thrombolytic therapy for acute myocardial infarction.

The term “hypertension”, as used herein, is selected, but not limited to, the group consisting of lipid disorders with hypertension, systolic hypertension and diastolic hypertension.

The term “peripheral vascular disease”, as used herein, is selected, but not limited to, the group consisting of peripheral vascular disease and claudication.

The term “diabetes”, as used herein, refers to any of a number of diabetogenic states including type I diabetes, type II diabetes, Syndrome X, Metabolic syndrome, lipid disorders associated with insulin resistance, impaired glucose tolerance, non-insulin dependent diabetes, microvascular diabetic complications, reduced nerve conduction velocity, reduced or loss of vision, diabetic retinopathy, increased risk of amputation, decreased kidney function, kidney failure, insulin resistance syndrome, pluri-metabolic syndrome, central adiposity (visceral) (upper body), diabetic dyslipidemia, decreased insulin sensitization, diabetic retinopathy/neuropathy, diabetic nephropathy/micro and macro angiopathy and micro/macro albuminuria, diabetic cardiomyopathy, diabetic gastroparesis, obesity, increased hemoglobin glycosilation (including HbAlC), improved glucose control, impaired renal function (dialysis, endstage) and hepatic function (mild, moderate, severe).

“Metabolic syndrome,” also known as “Syndrome X,” refers to a common clinical disorder that is defined as the presence of increased insulin concentrations in association with other disorders including viceral obesity, hyperlipidemia, dyslipidemia, hyperglycemia, hypertension, and potentially hyperuricemis and renal dysfunction.

The carbon atom content of various hydrocarbon-containing moieties is indicated by a prefix designating the minimum and maximum number of carbon atoms in the moiety, i.e., the prefix Cᵢ-Cⱼ indicates a moiety of the integer “i” to the integer “j” carbon atoms, inclusive. Thus, for example, C₁-C₃ alkyl refers to alkyl of one to three carbon atoms, inclusive, or methyl, ethyl, propyl and isopropyl, and all isomeric forms and straight and branched forms thereof.

By “halo” or “halogen” is meant chloro, bromo, iodo, or fluoro.

By “alkyl” is meant straight chain saturated hydrocarbon or branched chain saturated hydrocarbon. Exemplary of such alkyl groups (assuming the designated length encompasses the particular example) are methyl, ethyl, propyl, isopropyl, butyl, sec-butyl, tertiary butyl, pentyl, isopentyl, neopentyl, tertiary pentyl, 1-methylbutyl, 2-methylbutyl, 3-methylbutyl, hexyl, isohexyl, heptyl and octyl. This term also includes a saturated hydrocarbon (straight chain or branched) wherein a hydrogen atom is removed from each of the terminal carbons.

“Alkenyl” referred to herein may be linear or branched, and they may also be cyclic (e.g. cyclobutenyl, cyclopentenyl, cyclohexenyl) or bicyclic or contain cyclic groups. They contain 1-3 carbon-carbon double bonds, which can be cis or trans.

By “alkoxy” is meant straight chain saturated alkyl or branched chain saturated alkyl bonded through an oxy. Exemplary of such alkoxy groups (assuming the designated length encompasses the particular example) are methoxy, ethoxy, propoxy, isopropoxy, butoxy, isobutoxy, tertiary butoxy, pentoxy, isopentoxy, neopentoxy, tertiary pentoxy, hexoxy, isohexoxy, heptoxy and octoxy.

Certain processes for the manufacture of the compounds of this invention are provided as further features of the invention and are illustrated by the following exemplary reaction schemes. Those skilled in the art will appreciate that other synthetic routes may be used to synthesize the inventive compounds. For a more detailed description of the individual reaction steps, see the Examples section below. Although specific starting materials and reagents are depicted in the schemes and discussed below, other starting materials and reagents can be easily substituted to provide a variety of derivatives and/or reaction conditions. In addition, many of the compounds prepared by the methods described below can be further modified in light of this disclosure using conventional chemistry well known to those skilled in the art. In particular, it is noted that the compounds prepared according to these Schemes may be modified further to provide new Examples within the scope of this invention. In addition, it will be evident from the detailed descriptions given in the Experimental section that the modes of preparation employed extend further than the general procedures described herein.

The starting materials are generally available from commercial sources such as Aldrich Chemicals (Milwaukee, Wis.) or are readily prepared using methods known to those skilled in the art (e.g., prepared by methods generally described in Louis F. Fieser and Mary Fieser, Reagents for Organic Synthesis, v. 1-19, Wiley, New York (1967-1999 ed.), or Beilsteins Handbuch der organischen Chemie, 4, Aufl. ed. Springer-Verlag, Berlin, including supplements (also available via the Beilstein online database).

As an initial note, in the preparation of compounds of the present invention, it is noted that some of the preparation methods useful for the preparation of the compounds described herein may require protection of remote functionality (e.g., primary amine, secondary amine, carboxyl in intermediates). The need for such protection will vary depending on the nature of the remote functionality and the conditions of the preparative methods and can be readily determined by one of ordinary skill in the art. The use of such protection/deprotection methods is also within the ordinary skill in the art. For a general description of protecting groups and their use, see T. W. Greene, Protective Groups in Organic Synthesis, John Wiley & Sons, New York, 1991.

For example, in the reaction schemes below, certain compounds contain primary amines or carboxylic acid functionalities, which may interfere with reactions at other sites of the molecule if left unprotected. Accordingly, such functionalities may be protected by an appropriate protecting group, which may be removed in a subsequent step. Suitable protecting groups for amine and carboxylic acid protection include those protecting groups commonly used in peptide synthesis (such as N-t-butoxycarbonyl, benzyloxycarbonyl, and 9-fluorenylmethylenoxycarbonyl for amines and lower alkyl or benzyl esters for carboxylic acids) which are generally not chemically reactive under the reaction conditions described and can typically be removed without chemically altering other functionality in the compound.

The schemes below, while depicting racemic mixtures, can be used to synthesize individual enantiomers by starting with the appropriate chiral starting materials.

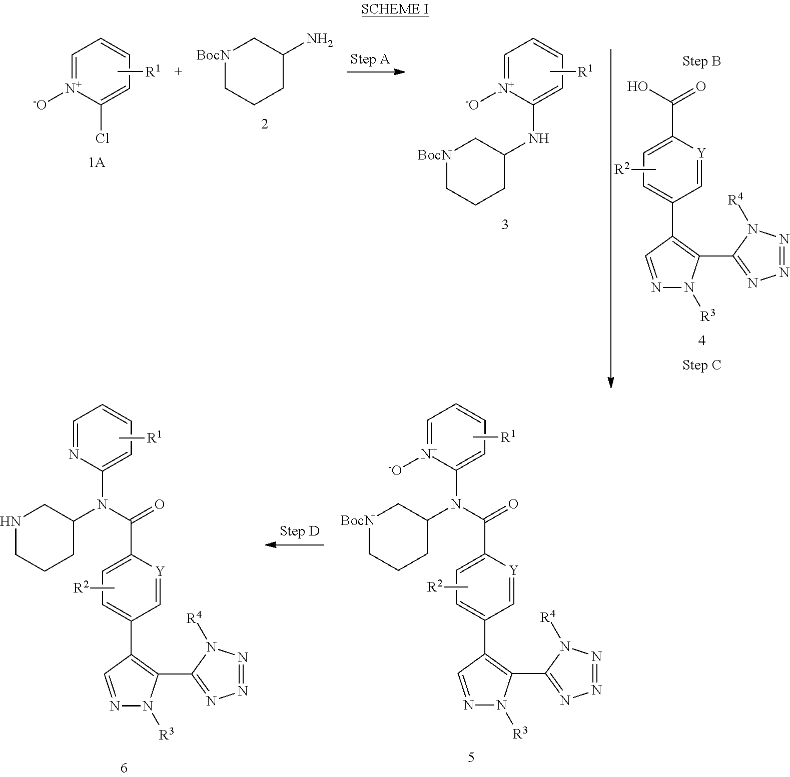

Compounds of Formula I, wherein R¹, R², R³ and Y are as defined above and R⁴ is H are prepared as depicted in Scheme I above. In Step A, the Formula 2 amine and Formula 1A N-oxide (readily obtained from commercial sources) are preferably reacted in the presence of a base such diisopropylethylamine, triethylamine (optionally with an additive such as cesium fluoride), potassium acetate, cesium carbonate, or other carbonate sources in solvents such as dimethylsulfoxide (DMSO), acetonitrile, or isopropanol at a temperature of about 20° C. to about 160° C. for about 1 hour to about 24 hours resulting in the Formula 3 N-oxide. In Step B, Formula 4 carboxylic acid and Formula 3 N-oxide are reacted to provide the Formula 5 compound (Londregan, A. T. et al Tetrahedron Lett., 2009, 1986-1988). The reaction preferably proceeds with an activating agent such as oxalyl chloride, benzotriazol-1-yloxy-tris(dimethylamino)-phosphonium hexafluorophosphate (BOP), bromo-tris-pyrrolidino phosphoniumhexafluorophosphate (PyBrOP), or suitable substitute in solvents such as dichloromethane, 1,4-dioxane, tetrahydrofuran (THF), acetonitrile, and DMF at a temperature of about 0° C. to about 50° C. for about 0.5 hours to about 24 hours. In addition, Step B is carried out in the presence of additives such as diisopropylethylamine, triethylamine, 2,6-lutidine or similar bases. The Formula 4 acid R², R³ and Y substituents are selected to provide the desired Formula I substituents, or the R², R³, R⁴ and Y substituents can be modified after addition by procedures known in the chemical art to obtain alternative Formula I R², R³ and R⁴ substituents. Step C includes a one pot reduction of the Formula 5 N-oxide, followed by cleavage (Step D) of the t-butoxycarbonyl group (BOC). The t-butoxycarbonyl (BOC) is cleaved in Step D with acids such as hydrochloric acid (HCl), trifluoracetic acid (TFA), p-toluene sulfonic acid in aqueous or non-aqueous (e.g. dichloromethane, tetrahydrofuran, ethyl acetate, toluene) conditions at a temperature of about 0° C. to about 50° C. for about 0.5 hours to about 18 hours. Those skilled in the art will recognize that a variety of other conditions may be used to cleave the t-butoxycarbonyl (BOC) group.

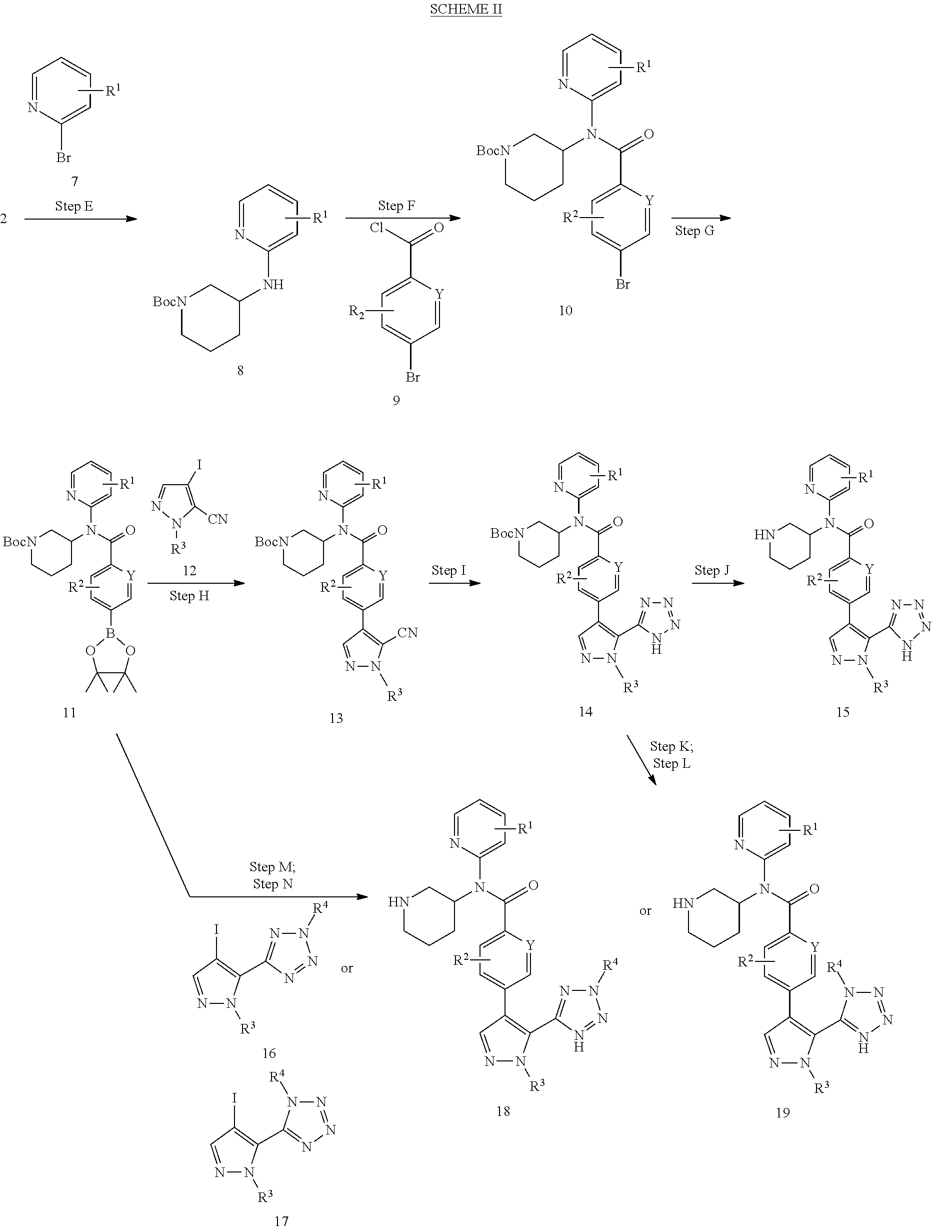

Formula I compounds can also be prepared according to Scheme II. Step E is preferably carried out with a Formula 2 amine and a Formula 7 aryl bromide in the presence of a palladium catalyst, or precatalyst and ligand (e.g. 2-(dimethylaminomethyl)ferrocen-1-yl-palladium(II) chloride dinorbornylphosphine, palladium acetate (Pd(OAc)₂), Brettphos, PEPPSI™, Josiphos, BINAP) or other suitable catalysts. The reaction proceeds at a temperature of about 90° C. to about 150° C. for about 1 hour to about 24 hours in solvents such as methanol, ethanol, water, acetonitrile, N,N-dimethylformamide (DMF), 1,4-dioxane, and THF. Exemplary bases for this reaction are potassium t-butoxide (KOt-Bu) and cesium carbonate (Cs₂CO₃). In Step F the Formula 10 compound is synthesized by deprotonation of the Formula 8 protected amine with a strong base such as methylmagnesium chloride (MeMgCl), n-butyllithium (n-BuLi), lithium N,N-diisopropylamine, lithium hexamethyldisilazide (LiHMDS) or other similar bases in solvents such THF, 1,4-dioxane, or 1,2-dimethoxyethane (DME) at a temperature of about −78° C. to about 23° C. for about 1 hour to about 4 hours. Addition of the Formula 9 acyl chloride at a temperature of about −10° C. to about 23° C. for about 1 hour to about 18 hours yields the Formula 10 compound. The Formula 9 acyl chloride is commercially available or synthesized using methods known to those skilled in the chemical arts.

Step G is preferably carried out with a suitable boronate source, such as bis(pinacolato)diboron in the presence of a palladium compound (e.g. tris(dibenzylideneacetone)dipalladium (Pd₂(dba)₃), 1,1-bis(diphenylphosphino)ferrocene]dichloropalladium(II) (PdCl₂(dppf)₂), tetrakis(triphenylphosphine)palladium (Pd(PPh₃)₄) or other suitable catalysts. The reaction proceeds at a temperature of about 23° C. to about 180° C. for about 1 hour to about 24 hours. Exemplary solvents for Step G are methanol, ethanol, water, acetonitrile, N,N-dimethylformamide (DMF), 1,4-dioxane, and tetrahydrofuran (THF). Step G is carried out in the presence of a base such as potassium acetate (KOAc), cesium carbonate (Cs₂CO₃), sodium hydroxide, (NaOH), potassium hydroxide (KOH), potassium or sodium carbonate and sodium bicarbonate (K₂CO₃, Na₂CO₃, NaHCO₃).

In Step H, Formula 11 boronate and a Formula 12 pyrazole are combined via a cross-coupling reaction under conditions similar to those used in Step G. The Formula 12 cyano-pyrazole R³ substituent is selected to provide the desired Formula I substituents, or the R² and R³ substituents can be modified after addition by procedures known in the chemical art.

In Step I, the Formula 13 cyano-pyrazole is converted into a tetrazole derivative by procedures known in the chemical arts. Conditions for this transformation include but are not limited to the reaction of a cyano derivative with an inorganic, organometallic, or organosilicon azide source with or without a Lewis or Brønsted acid (Roh et al, Eur. J. Org. Chem. 2012, 6101-6118 and pertinent references therein). In Step J, compounds of Formula 14 are subjected to acidic conditions, as described in Scheme I Step D, to remove the t-butoxycarbonyl (BOC) group. Alternatively, compounds of Formula 14 can be further derivatized in Step K, followed by cleavage of the t-butoxycarbonyl group to give Formula I compounds. In Step K, reactions of the Formula 14 compound with alkylating agents produce the two regioisomers of Formula 18 and 19 shown in Scheme II. In Step L, the t-butoxycarbonyl group is then removed as in Scheme I Step D to provide compounds of Formula I as described above. These regioisomers can be used as a single pharmaceutical ingredient or used as two separate and distinct pharmaceutical ingredients. Compounds of Formula 18 and 19 can also be prepared by reacting compounds of Formula 11 with Formula 16 or Formula 17 compounds in Step M, using conditions similar to those in Step H, followed by Step N, as described in Scheme I Step D, to provide the two regioisomers of Formula 18 and 19.

After the reaction is completed, the desired Formula I compound, exemplified in the above schemes may be recovered and isolated as known in the art. It may be recovered by evaporation and/or extraction as is known in the art. It may optionally be purified by chromatography, recrystallization, distillation, or other techniques known in the art.

The Formula I compounds of this invention may also be used in conjunction with other pharmaceutical agents (e.g., LDL-cholesterol lowering agents, triglyceride lowering agents) for the treatment of the disease/conditions described herein. For example, they may be used in combination with lipid modulating agents, antidiabetic agents and cardiovascular agents.

Lipid modulating agents may be used as a combination agent in conjunction with the Formula I compounds. Any HMG-CoA reductase inhibitor may be used in the combination aspect of this invention. The conversion of 3-hydroxy-3-methylglutaryl-coenzyme A (HMG-CoA) to mevalonate is an early and rate-limiting step in the cholesterol biosynthetic pathway. This step is catalyzed by the enzyme HMG-CoA reductase. Statins inhibit HMG-CoA reductase from catalyzing this conversion. The following paragraphs describe exemplary statins.

The term HMG-CoA reductase inhibitor refers to compounds which inhibit the bioconversion of hydroxymethylglutaryl-coenzyme A to mevalonic acid catalyzed by the enzyme HMG-CoA reductase. Such inhibition is readily determined by those skilled in the art according to standard assays (e.g., Meth. Enzymol. 1981; 71:455-509 and references cited therein). A variety of these compounds are described and referenced below however other HMG-CoA reductase inhibitors will be known to those skilled in the art. U.S. Pat. No. 4,231,938 (the disclosure of which is hereby incorporated by reference) discloses certain compounds isolated after cultivation of a microorganism belonging to the genus Aspergillus, such as lovastatin. Also, U.S. Pat. No. 4,444,784 (the disclosure of which is hereby incorporated by reference) discloses synthetic derivatives of the aforementioned compounds, such as simvastatin. Also, U.S. Pat. No. 4,739,073 (the disclosure of which is incorporated by reference) discloses certain substituted indoles, such as fluvastatin. Also, U.S. Pat. No. 4,346,227 (the disclosure of which is incorporated by reference) discloses ML-236B derivatives, such as pravastatin. Also, EP-491226A (the disclosure of which is incorporated by reference) discloses certain pyridyldihydroxyheptenoic acids, such as cerivastatin. In addition, U.S. Pat. No. 5,273,995 (the disclosure of which is incorporated by reference) discloses certain 6-[2-(substituted-pyrrol-1-yl)alkyl]pyran-2-ones such as atorvastatin and any pharmaceutically acceptable form thereof (i.e. LIPITOR®). Additional HMG-CoA reductase inhibitors include rosuvastatin and pitavastatin.

Atorvastatin calcium (i.e., atorvastatin hemicalcium), disclosed in U.S. Pat. No. 5,273,995, which is incorporated herein by reference, is currently sold as Lipitor®.

Statins also include such compounds as rosuvastatin disclosed in U.S. RE37,314 E, pitivastatin disclosed in EP 304063 B1 and U.S. Pat. No. 5,011,930, simvastatin, disclosed in U.S. Pat. No. 4,444,784, which is incorporated herein by reference; pravastatin, disclosed in U.S. Pat. No. 4,346,227 which is incorporated herein by reference; cerivastatin, disclosed in U.S. Pat. No. 5,502,199, which is incorporated herein by reference; mevastatin, disclosed in U.S. Pat. No. 3,983,140, which is incorporated herein by reference; velostatin, disclosed in U.S. Pat. No. 4,448,784 and U.S. Pat. No. 4,450,171, both of which are incorporated herein by reference; fluvastatin, disclosed in U.S. Pat. No. 4,739,073, which is incorporated herein by reference; compactin, disclosed in U.S. Pat. No. 4,804,770, which is incorporated herein by reference; lovastatin, disclosed in U.S. Pat. No. 4,231,938, which is incorporated herein by reference; dalvastatin, disclosed in European Patent Application Publication No. 738510 A2; fluindostatin, disclosed in European Patent Application Publication No. 363934 A1; and dihydrocompactin, disclosed in U.S. Pat. No. 4,450,171, which is incorporated herein by reference.

Any HMG-CoA synthase inhibitor may be used in the combination aspect of this invention. The term HMG-CoA synthase inhibitor refers to compounds which inhibit the biosynthesis of hydroxymethylglutaryl-coenzyme A from acetyl-coenzyme A and acetoacetyl-coenzyme A, catalyzed by the enzyme HMG-CoA synthase. Such inhibition is readily determined by those skilled in the art according to standard assays (Meth Enzymol. 1975; 35:155-160: Meth. Enzymol. 1985; 110:19-26 and references cited therein). A variety of these compounds are described and referenced below, however other HMG-CoA synthase inhibitors will be known to those skilled in the art. U.S. Pat. No. 5,120,729 (the disclosure of which is hereby incorporated by reference) discloses certain beta-lactam derivatives. U.S. Pat. No. 5,064,856 (the disclosure of which is hereby incorporated by reference) discloses certain spiro-lactone derivatives prepared by culturing a microorganism (MF5253). U.S. Pat. No. 4,847,271 (the disclosure of which is hereby incorporated by reference) discloses certain oxetane compounds such as 11-(3-hydroxymethyl-4-oxo-2-oxetayl)-3,5,7-trimethyl-2,4-undeca-dienoic acid derivatives.

Any compound that decreases HMG-CoA reductase gene expression may be used in the combination aspect of this invention. These agents may be HMG-CoA reductase transcription inhibitors that block the transcription of DNA or translation inhibitors that prevent or decrease translation of mRNA coding for HMG-CoA reductase into protein. Such compounds may either affect transcription or translation directly, or may be biotransformed to compounds that have the aforementioned activities by one or more enzymes in the cholesterol biosynthetic cascade or may lead to the accumulation of an isoprene metabolite that has the aforementioned activities. Such compounds may cause this effect by decreasing levels of SREBP (sterol regulatory element binding protein) by inhibiting the activity of site-1 protease (S1P) or agonizing the oxysterol receptor or antagonizing SOAP. Such regulation is readily determined by those skilled in the art according to standard assays (Meth. Enzymol. 1985; 110:9-19). Several compounds are described and referenced below, however other inhibitors of HMG-CoA reductase gene expression will be known to those skilled in the art. U.S. Pat. No. 5,041,432 (the disclosure of which is incorporated by reference) discloses certain 15-substituted lanosterol derivatives.

Other oxygenated sterols that suppress synthesis of HMG-CoA reductase are discussed by E. I. Mercer (Prog. Lip. Res. 1993; 32:357-416).

Any compound having activity as a CETP inhibitor can serve as the second compound in the combination therapy aspect of the present invention. The term CETP inhibitor refers to compounds that inhibit the cholesteryl ester transfer protein (CETP) mediated transport of various cholesteryl esters and triglycerides from HDL to LDL and VLDL. Such CETP inhibition activity is readily determined by those skilled in the art according to standard assays (e.g., U.S. Pat. No. 6,140,343). A variety of CETP inhibitors will be known to those skilled in the art, for example, those disclosed in commonly assigned U.S. Pat. No. 6,140,343 and commonly assigned U.S. Pat. No. 6,197,786. CETP inhibitors are also described in U.S. Pat. No. 6,723,752, which includes a number of CETP inhibitors including (2R)-3-{[3-(4-Chloro-3-ethyl-phenoxy)-phenyl]-[[3-(1,1,2,2-tetrafluoro-ethoxy)-phenyl]-methyl]-amino}-1,1,1-trifluoro-2-propanol. Moreover, CETP inhibitors included herein are also described in U.S. patent application Ser. No. 10/807,838 filed Mar. 23, 2004. U.S. Pat. No. 5,512,548 discloses certain polypeptide derivatives having activity as CETP inhibitors, while certain CETP-inhibitory rosenonolactone derivatives and phosphate-containing analogs of cholesteryl ester are disclosed in J. Antibiot., 49(8): 815-816 (1996), and Bioorg. Med. Chem. Lett.; 6:1951-1954 (1996), respectively.

Any PPAR modulator may be used in the combination aspect of this invention. The term PPAR modulator refers to compounds which modulate peroxisome proliferator activator receptor (PPAR) activity in mammals, particularly humans. Such modulation is readily determined by those skilled in the art according to standard assays known in the literature. It is believed that such compounds, by modulating the PPAR receptor, regulate transcription of key genes involved in lipid and glucose metabolism such as those in fatty acid oxidation and also those involved in high density lipoprotein (HDL) assembly (for example, apolipoprotein Al gene transcription), accordingly reducing whole body fat and increasing HDL cholesterol. By virtue of their activity, these compounds also reduce plasma levels of triglycerides, VLDL cholesterol, LDL cholesterol and their associated components such as apolipoprotein B in mammals, particularly humans, as well as increasing HDL cholesterol and apolipoprotein A1. Hence, these compounds are useful for the treatment and correction of the various dyslipidemias observed to be associated with the development and incidence of atherosclerosis and cardiovascular disease, including hypoalphalipoproteinemia and hypertriglyceridemia. A variety of these compounds are described and referenced below, however, others will be known to those skilled in the art. International Publication Nos. WO 02/064549 and 02/064130 and U.S. patent application Ser. No. 10/720,942, filed Nov. 24, 2003 and U.S. patent application 60/552,114 filed Mar. 10, 2004 (the disclosures of which are hereby incorporated by reference) disclose certain compounds which are PPARa activators.

Any other PPAR modulator may be used in the combination aspect of this invention. In particular, modulators of PPARβ and/or PPARγ may be useful in combination with compounds of the present invention. An example PPAR inhibitor is described in US2003/0225158 as {5-Methoxy-2-methyl-4-[4-(4-trifluoromethyl-benzyloxy)-benzylsulfany]-phenoxy}-acetic acid.

Any MTP/Apo B (microsomal triglyceride transfer protein and or apolipoprotein B) secretion inhibitor may be used in the combination aspect of this invention. The term MTP/Apo B secretion inhibitor refers to compounds which inhibit the secretion of triglycerides, cholesteryl ester, and phospholipids. Such inhibition is readily determined by those skilled in the art according to standard assays (e.g., Wetterau, J. R. 1992; Science 258:999). A variety of MTP/Apo B secretion inhibitors will be known to those skilled in the art, including imputapride (Bayer) and additional compounds such as those disclosed in WO 96/40640 and WO 98/23593, (two exemplary publications).

Any squalene synthetase inhibitor may be used in the combination aspect of this invention. The term squalene synthetase inhibitor refers to compounds which inhibit the condensation of 2 molecules of farnesylpyrophosphate to form squalene, catalyzed by the enzyme squalene synthetase. Such inhibition is readily determined by those skilled in the art according to standard assays (Meth. Enzymol. 1969; 15: 393-454 and Meth. Enzymol. 1985; 110:359-373 and references contained therein). A variety of these compounds are described in and referenced below however other squalene synthetase inhibitors will be known to those skilled in the art. U.S. Pat. No. 5,026,554 (the disclosure of which is incorporated by reference) discloses fermentation products of the microorganism MF5465 (ATCC 74011) including zaragozic acid. A summary of other patented squalene synthetase inhibitors has been compiled (Curr. Op. Ther. Patents (1993) 861-4).

Any squalene epoxidase inhibitor may be used in the combination aspect of this invention. The term squalene epoxidase inhibitor refers to compounds which inhibit the bioconversion of squalene and molecular oxygen into squalene-2,3-epoxide, catalyzed by the enzyme squalene epoxidase. Such inhibition is readily determined by those skilled in the art according to standard assays (Biochim. Biophys. Acta 1984; 794:466-471). A variety of these compounds are described and referenced below, however other squalene epoxidase inhibitors will be known to those skilled in the art. U.S. Pat. Nos. 5,011,859 and 5,064,864 (the disclosures of which are incorporated by reference) disclose certain fluoro analogs of squalene. EP publication 395,768 A (the disclosure of which is incorporated by reference) discloses certain substituted allylamine derivatives. PCT publication WO 9312069 A (the disclosure of which is hereby incorporated by reference) discloses certain amino alcohol derivatives. U.S. Pat. No. 5,051,534 (the disclosure of which is hereby incorporated by reference) discloses certain cyclopropyloxy-squalene derivatives.

Any squalene cyclase inhibitor may be used as the second component in the combination aspect of this invention. The term squalene cyclase inhibitor refers to compounds which inhibit the bioconversion of squalene-2,3-epoxide to lanosterol, catalyzed by the enzyme squalene cyclase. Such inhibition is readily determined by those skilled in the art according to standard assays (FEBS Lett. 1989; 244:347-350). In addition, the compounds described and referenced below are squalene cyclase inhibitors, however other squalene cyclase inhibitors will also be known to those skilled in the art. PCT publication WO9410150 (the disclosure of which is hereby incorporated by reference) discloses certain 1,2,3,5,6,7,8,8a-octahydro-5,5,8(beta)-trimethyl-6-isoquinolineamine derivatives, such as N-trifluoroacetyl-1,2,3,5,6,7,8,8a-octahydro-2-allyl-5,5,8(beta)-trimethyl-6(beta)-isoquinolineamine. French patent publication 2697250 (the disclosure of which is hereby incorporated by reference) discloses certain beta, beta-dimethyl-4-piperidine ethanol derivatives such as 1-(1,5,9-trimethyldecyl)-beta,beta-dimethyl-4-piperidineethanol

Any combined squalene epoxidase/squalene cyclase inhibitor may be used as the second component in the combination aspect of this invention. The term combined squalene epoxidase/squalene cyclase inhibitor refers to compounds that inhibit the bioconversion of squalene to lanosterol via a squalene-2,3-epoxide intermediate. In some assays it is not possible to distinguish between squalene epoxidase inhibitors and squalene cyclase inhibitors; however, these assays are recognized by those skilled in the art. Thus, inhibition by combined squalene epoxidase/squalene cyclase inhibitors is readily determined by those skilled in art according to the aforementioned standard assays for squalene cyclase or squalene epoxidase inhibitors. A variety of these compounds are described and referenced below, however other squalene epoxidase/squalene cyclase inhibitors will be known to those skilled in the art. U.S. Pat. Nos. 5,084,461 and 5,278,171 (the disclosures of which are incorporated by reference) disclose certain azadecalin derivatives. EP publication 468,434 (the disclosure of which is incorporated by reference) discloses certain piperidyl ether and thio-ether derivatives such as 2-(1-piperidyl)pentyl isopentyl sulfoxide and 2-(1-piperidyl)ethyl ethyl sulfide. PCT publication WO 9401404 (the disclosure of which is hereby incorporated by reference) discloses certain acyl-piperidines such as 1-(1-oxopentyl-5-phenylthio)-4-(2-hydroxy-1-methyl)-ethyl)piperidine. U.S. Pat. No. 5,102,915 (the disclosure of which is hereby incorporated by reference) discloses certain cyclopropyloxy-squalene derivatives.

The compounds of the present invention can also be administered in combination with naturally occurring compounds that act to lower plasma cholesterol levels. These naturally occurring compounds are commonly called nutraceuticals and include, for example, garlic extract and niacin. A slow-release form of niacin is available and is known as Niaspan. Niacin may also be combined with other therapeutic agents such as lovastatin, or another HMG-CoA reductase inhibitor. This combination therapy with lovastatin is known as ADVICOR™ (Kos Pharmaceuticals Inc.).

Any cholesterol absorption inhibitor can be used as an additional compound in the combination aspect of the present invention. The term cholesterol absorption inhibition refers to the ability of a compound to prevent cholesterol contained within the lumen of the intestine from entering into the intestinal cells and/or passing from within the intestinal cells into the lymph system and/or into the blood stream. Such cholesterol absorption inhibition activity is readily determined by those skilled in the art according to standard assays (e.g., J. Lipid Res. (1993) 34: 377-395). Cholesterol absorption inhibitors are known to those skilled in the art and are described, for example, in PCT WO 94/00480. An example of a cholesterol absorption inhibitor is ZETIA™ (ezetimibe) (Schering-Plough/Merck).

Any ACAT inhibitor may be used in the combination therapy aspect of the present invention. The term ACAT inhibitor refers to compounds that inhibit the intracellular esterification of dietary cholesterol by the enzyme acyl CoA: cholesterol acyltransferase. Such inhibition may be determined readily by one of skill in the art according to standard assays, such as the method of Heider et al. described in Journal of Lipid Research., 24:1127 (1983). A variety of these compounds are known to those skilled in the art, for example, U.S. Pat. No. 5,510,379 discloses certain carboxysulfonates, while WO 96/26948 and WO 96/10559 both disclose urea derivatives having ACAT inhibitory activity. Examples of ACAT inhibitors include compounds such as Avasimibe (Pfizer), CS-505 (Sankyo) and Eflucimibe (Eli Lilly and Pierre Fabre).

A lipase inhibitor may be used in the combination therapy aspect of the present invention. A lipase inhibitor is a compound that inhibits the metabolic cleavage of dietary triglycerides or plasma phospholipids into free fatty acids and the corresponding glycerides (e.g. EL, HL, etc.). Under normal physiological conditions, lipolysis occurs via a two-step process that involves acylation of an activated serine moiety of the lipase enzyme. This leads to the production of a fatty acid-lipase hemiacetal intermediate, which is then cleaved to release a diglyceride. Following further deacylation, the lipase-fatty acid intermediate is cleaved, resulting in free lipase, a glyceride and fatty acid. In the intestine, the resultant free fatty acids and monoglycerides are incorporated into bile acid-phospholipid micelles, which are subsequently absorbed at the level of the brush border of the small intestine. The micelles eventually enter the peripheral circulation as chylomicrons. Such lipase inhibition activity is readily determined by those skilled in the art according to standard assays (e.g., Methods Enzymol. 286: 190-231).

Pancreatic lipase mediates the metabolic cleavage of fatty acids from triglycerides at the 1- and 3-carbon positions. The primary site of the metabolism of ingested fats is in the duodenum and proximal jejunum by pancreatic lipase, which is usually secreted in vast excess of the amounts necessary for the breakdown of fats in the upper small intestine. Because pancreatic lipase is the primary enzyme required for the absorption of dietary triglycerides, inhibitors have utility in the treatment of obesity and the other related conditions. Such pancreatic lipase inhibition activity is readily determined by those skilled in the art according to standard assays (e.g., Methods Enzymol. 286: 190-231).

Gastric lipase is an immunologically distinct lipase that is responsible for approximately 10 to 40% of the digestion of dietary fats. Gastric lipase is secreted in response to mechanical stimulation, ingestion of food, the presence of a fatty meal or by sympathetic agents. Gastric lipolysis of ingested fats is of physiological importance in the provision of fatty acids needed to trigger pancreatic lipase activity in the intestine and is also of importance for fat absorption in a variety of physiological and pathological conditions associated with pancreatic insufficiency. See, for example, C. K. Abrams, et al., Gastroenterology, 92, 125 (1987). Such gastric lipase inhibition activity is readily determined by those skilled in the art according to standard assays (e.g., Methods Enzymol. 286: 190-231).

A variety of gastric and/or pancreatic lipase inhibitors are known to one of ordinary skill in the art. Preferred lipase inhibitors are those inhibitors that are selected from the group consisting of lipstatin, tetrahydrolipstatin (orlistat), valilactone, esterastin, ebelactone A, and ebelactone B. The compound tetrahydrolipstatin is especially preferred. The lipase inhibitor, N-3-trifluoromethylphenyl-N′-3-chloro-4′-trifluoromethylphenylurea, and the various urea derivatives related thereto, are disclosed in U.S. Pat. No. 4,405,644. The lipase inhibitor, esteracin, is disclosed in U.S. Pat. Nos. 4,189,438 and 4,242,453. The lipase inhibitor, cyclo-O,O′-[(1,6-hexanediyl)-bis-(iminocarbonyl)]dioxime, and the various bis(iminocarbonyl)dioximes related thereto may be prepared as described in Petersen et al., Liebig's Annalen, 562, 205-229 (1949).

A variety of pancreatic lipase inhibitors are described herein below. The pancreatic lipase inhibitors lipstatin, (2S,3S,5S,7Z,10Z)-5-[(S)-2-formamido-4-methyl-valeryloxy]-2-hexyl-3-hydroxy-7,10-hexadecanoic acid lactone, and tetrahydrolipstatin (orlistat), (2S,3S,5S)-5-[(S)-2-formamido-4-methyl-valeryloxy]-2-hexyl-3-hydroxy-hexadecanoic 1,3 acid lactone, and the variously substituted N-formylleucine derivatives and stereoisomers thereof, are disclosed in U.S. Pat. No. 4,598,089. For example, tetrahydrolipstatin is prepared as described in, e.g., U.S. Pat. Nos. 5,274,143; 5,420,305; 5,540,917; and 5,643,874. The pancreatic lipase inhibitor, FL-386, 1-[4-(2-methylpropyl)cyclohexyl]-2-[(phenylsulfonyl)oxy]-ethanone, and the variously substituted sulfonate derivatives related thereto, are disclosed in U.S. Pat. No. 4,452,813. The pancreatic lipase inhibitor, WAY-121898, 4-phenoxyphenyl-4-methylpiperidin-1-yl-carboxylate, and the various carbamate esters and pharmaceutically acceptable salts related thereto, are disclosed in U.S. Pat. Nos. 5,512,565; 5,391,571 and 5,602,151. The pancreatic lipase inhibitor, valilactone, and a process for the preparation thereof by the microbial cultivation of Actinomycetes strain MG147-CF2, are disclosed in Kitahara, et al., J. Antibiotics, 40 (11), 1647-1650 (1987). The pancreatic lipase inhibitors, ebelactone A and ebelactone B, and a process for the preparation thereof by the microbial cultivation of Actinomycetes strain MG7-G1, are disclosed in Umezawa, et al., J. Antibiotics, 33, 1594-1596 (1980). The use of ebelactones A and B in the suppression of monoglyceride formation is disclosed in Japanese Kokai 08-143457, published Jun. 4, 1996.

Other compounds that are marketed for hyperlipidemia, including hypercholesterolemia and which are intended to help prevent or treat atherosclerosis include bile acid sequestrants, such as Welchol®, Colestid®, LoCholest® and Questran®; and fibric acid derivatives, such as Atromid®, Lopid® and Tricor®.

Given the association between diabetes and atherosclerosis (e.g., Metabolic Syndrome) the compounds of formula I may be administered with antidiabetic compounds. Diabetes can be treated by administering to a patient having diabetes (especially Type II), insulin resistance, impaired glucose tolerance, metabolic syndrome, or the like, or any of the diabetic complications such as neuropathy, nephropathy, retinopathy or cataracts, a therapeutically effective amount of a compound of the present invention in combination with other agents (e.g., insulin) that can be used to treat diabetes. This includes the classes of anti-diabetic agents (and specific agents) described herein.

Any glycogen phosphorylase inhibitor can be used as the second agent in combination with a compound of the present invention. The term glycogen phosphorylase inhibitor refers to compounds that inhibit the bioconversion of glycogen to glucose-1-phosphate which is catalyzed by the enzyme glycogen phosphorylase. Such glycogen phosphorylase inhibition activity is readily determined by those skilled in the art according to standard assays (e.g., J. Med. Chem. 41 (1998) 2934-2938). A variety of glycogen phosphorylase inhibitors are known to those skilled in the art including those described in WO 96/39384 and WO 96/39385.

Any aldose reductase inhibitor can be used in combination with a compound of the present invention. The term aldose reductase inhibitor refers to compounds that inhibit the bioconversion of glucose to sorbitol, which is catalyzed by the enzyme aldose reductase. Aldose reductase inhibition is readily determined by those skilled in the art according to standard assays (e.g., J. Malone, Diabetes, 29:861-864 (1980). “Red Cell Sorbitol, an Indicator of Diabetic Control”). A variety of aldose reductase inhibitors are known to those skilled in the art, such as those described in U.S. Pat. No. 6,579,879, which includes 6-(5-chloro-3-methyl-benzofuran-2-sulfonyl)-2H-pyridazin-3-one.

Any sorbitol dehydrogenase inhibitor can be used in combination with a compound of the present invention. The term sorbitol dehydrogenase inhibitor refers to compounds that inhibit the bioconversion of sorbitol to fructose which is catalyzed by the enzyme sorbitol dehydrogenase. Such sorbitol dehydrogenase inhibitor activity is readily determined by those skilled in the art according to standard assays (e.g., Analyt. Biochem (2000) 280: 329-331). A variety of sorbitol dehydrogenase inhibitors are known, for example, U.S. Pat. Nos. 5,728,704 and 5,866,578 disclose compounds and a method for treating or preventing diabetic complications by inhibiting the enzyme sorbitol dehydrogenase.

Any glucosidase inhibitor can be used in combination with a compound of the present invention. A glucosidase inhibitor inhibits the enzymatic hydrolysis of complex carbohydrates by glycoside hydrolases, for example amylase or maltase, into bioavailable simple sugars, for example, glucose. The rapid metabolic action of glucosidases, particularly following the intake of high levels of carbohydrates, results in a state of alimentary hyperglycemia which, in adipose or diabetic subjects, leads to enhanced secretion of insulin, increased fat synthesis and a reduction in fat degradation. Following such hyperglycemias, hypoglycemia frequently occurs, due to the augmented levels of insulin present. Additionally, it is known chyme remaining in the stomach promotes the production of gastric juice, which initiates or favors the development of gastritis or duodenal ulcers. Accordingly, glucosidase inhibitors are known to have utility in accelerating the passage of carbohydrates through the stomach and inhibiting the absorption of glucose from the intestine. Furthermore, the conversion of carbohydrates into lipids of the fatty tissue and the subsequent incorporation of alimentary fat into fatty tissue deposits is accordingly reduced or delayed, with the concomitant benefit of reducing or preventing the deleterious abnormalities resulting therefrom. Such glucosidase inhibition activity is readily determined by those skilled in the art according to standard assays (e.g., Biochemistry (1969) 8: 4214).

A generally preferred glucosidase inhibitor includes an amylase inhibitor. An amylase inhibitor is a glucosidase inhibitor that inhibits the enzymatic degradation of starch or glycogen into maltose. Such amylase inhibition activity is readily determined by those skilled in the art according to standard assays (e.g., Methods Enzymol. (1955) 1: 149). The inhibition of such enzymatic degradation is beneficial in reducing amounts of bioavailable sugars, including glucose and maltose, and the concomitant deleterious conditions resulting therefrom.

A variety of glucosidase inhibitors are known to one of ordinary skill in the art and examples are provided below. Preferred glucosidase inhibitors are those inhibitors that are selected from the group consisting of acarbose, adiposine, voglibose, miglitol, emiglitate, camiglibose, tendamistate, trestatin, pradimicin-Q and salbostatin. The glucosidase inhibitor, acarbose, and the various amino sugar derivatives related thereto are disclosed in U.S. Pat. Nos. 4,062,950 and 4,174,439 respectively. The glucosidase inhibitor, adiposine, is disclosed in U.S. Pat. No. 4,254,256. The glucosidase inhibitor, voglibose, 3,4-dideoxy-4-[[2-hydroxy-1-(hydroxymethyl)ethyl]amino]-2-C-(hydroxymethyl)-D-epi-inositol, and the various N-substituted pseudo-aminosugars related thereto, are disclosed in U.S. Pat. No. 4,701,559. The glucosidase inhibitor, miglitol, (2R,3R,4R,5S)-1-(2-hydroxyethyl)-2-(hydroxymethyl)-3,4,5-piperidinetriol, and the various 3,4,5-trihydroxypiperidines related thereto, are disclosed in U.S. Pat. No. 4,639,436. The glucosidase inhibitor, emiglitate, ethyl p-[2-[(2R,3R,4R,5S)-3,4,5-trihydroxy-2-(hydroxymethyl)piperidino]ethoxy]-benzoate, the various derivatives related thereto and pharmaceutically acceptable acid addition salts thereof, are disclosed in U.S. Pat. No. 5,192,772. The glucosidase inhibitor, MDL-25637, 2,6-dideoxy-7-O-β-D-glucopyrano-syl-2,6-imino-D-glycero-L-gluco-heptitol, the various homodisaccharides related thereto and the pharmaceutically acceptable acid addition salts thereof, are disclosed in U.S. Pat. No. 4,634,765. The glucosidase inhibitor, camiglibose, methyl 6-deoxy-6-[(2R,3R,4R,5S)-3,4,5-trihydroxy-2-(hydroxymethyl)piperidino]-α-D-glucopyranoside sesquihydrate, the deoxy-nojirimycin derivatives related thereto, the various pharmaceutically acceptable salts thereof and synthetic methods for the preparation thereof, are disclosed in U.S. Pat. Nos. 5,157,116 and 5,504,078. The glycosidase inhibitor, salbostatin and the various pseudosaccharides related thereto, are disclosed in U.S. Pat. No. 5,091,524.

A variety of amylase inhibitors are known to one of ordinary skill in the art. The amylase inhibitor, tendamistat and the various cyclic peptides related thereto, are disclosed in U.S. Pat. No. 4,451,455. The amylase inhibitor AI-3688 and the various cyclic polypeptides related thereto are disclosed in U.S. Pat. No. 4,623,714. The amylase inhibitor, trestatin, consisting of a mixture of trestatin A, trestatin B and trestatin C and the various trehalose-containing aminosugars related thereto are disclosed in U.S. Pat. No. 4,273,765.

Additional anti-diabetic compounds, which can be used as the second agent in combination with a compound of the present invention, includes, for example, the following: biguanides (e.g., metformin), insulin secretagogues (e.g., sulfonylureas and glinides), glitazones, non-glitazone PPARγ agonists, PPARβ agonists, inhibitors of DPP-IV, inhibitors of PDE5, inhibitors of GSK-3, glucagon antagonists, inhibitors of f-1,6-BPase(Metabasis/Sankyo), GLP-1/analogs (AC 2993, also known as exendin-4), insulin and insulin mimetics (Merck natural products). Other examples would include PKC-β inhibitors and AGE breakers.

The compounds of the present invention can also be used in combination with cardiovascular agents such as antihypertensive agents. Any anti-hypertensive agent can be used as the second agent in such combinations and examples are provided herein. Such antihypertensive activity is readily determined by those skilled in the art according to standard assays (e.g., blood pressure measurements).

Amlodipine and related dihydropyridine compounds are disclosed in U.S. Pat. No. 4,572,909, which is incorporated herein by reference, as potent anti-ischemic and antihypertensive agents. U.S. Pat. No. 4,879,303, which is incorporated herein by reference, discloses amlodipine benzenesulfonate salt (also termed amlodipine besylate). Amlodipine and amlodipine besylate are potent and long lasting calcium channel blockers. As such, amlodipine, amlodipine besylate, amlodipine maleate and other pharmaceutically acceptable acid addition salts of amlodipine have utility as antihypertensive agents and as antiischemic agents. Amlodipine besylate is currently sold as Norvasc®.

Calcium channel blockers which are within the scope of this invention include, but are not limited to: bepridil, which may be prepared as disclosed in U.S. Pat. No. 3,962,238 or U.S. Reissue No. 30,577; clentiazem, which may be prepared as disclosed in U.S. Pat. No. 4,567,175; diltiazem, which may be prepared as disclosed in U.S. Pat. No. 3,562, fendiline, which may be prepared as disclosed in U.S. Pat. No. 3,262,977; gallopamil, which may be prepared as disclosed in U.S. Pat. No. 3,261,859; mibefradil, which may be prepared as disclosed in U.S. Pat. No. 4,808,605; prenylamine, which may be prepared as disclosed in U.S. Pat. No. 3,152,173; semotiadil, which may be prepared as disclosed in U.S. Pat. No. 4,786,635; terodiline, which may be prepared as disclosed in U.S. Pat. No. 3,371,014; verapamil, which may be prepared as disclosed in U.S. Pat. No. 3,261,859; aranipine, which may be prepared as disclosed in U.S. Pat. No. 4,572,909; barnidipine, which may be prepared as disclosed in U.S. Pat. No. 4,220,649; benidipine, which may be prepared as disclosed in European Patent Application Publication No. 106,275; cilnidipine, which may be prepared as disclosed in U.S. Pat. No. 4,672,068; efonidipine, which may be prepared as disclosed in U.S. Pat. No. 4,885,284; elgodipine, which may be prepared as disclosed in U.S. Pat. No. 4,952,592; felodipine, which may be prepared as disclosed in U.S. Pat. No. 4,264,611; isradipine, which may be prepared as disclosed in U.S. Pat. No. 4,466,972; lacidipine, which may be prepared as disclosed in U.S. Pat. No. 4,801,599; lercanidipine, which may be prepared as disclosed in U.S. Pat. No. 4,705,797; manidipine, which may be prepared as disclosed in U.S. Pat. No. 4,892,875; nicardipine, which may be prepared as disclosed in U.S. Pat. No. 3,985,758; nifedipine, which may be prepared as disclosed in U.S. Pat. No. 3,485,847; nilvadipine, which may be prepared as disclosed in U.S. Pat. No. 4,338,322; nimodipine, which may be prepared as disclosed in U.S. Pat. No. 3,799,934; nisoldipine, which may be prepared as disclosed in U.S. Pat. No. 4,154,839; nitrendipine, which may be prepared as disclosed in U.S. Pat. No. 3,799,934; cinnarizine, which may be prepared as disclosed in U.S. Pat. No. 2,882,271; flunarizine, which may be prepared as disclosed in U.S. Pat. No. 3,773,939; lidoflazine, which may be prepared as disclosed in U.S. Pat. No. 3,267,104; lomerizine, which may be prepared as disclosed in U.S. Pat. No. 4,663,325; bencyclane, which may be prepared as disclosed in Hungarian Patent No. 151,865; etafenone, which may be prepared as disclosed in German Patent No. 1,265,758; and perhexiline, which may be prepared as disclosed in British Patent No. 1,025,578. The disclosures of all such U.S. patents are incorporated herein by reference. Examples of presently marketed products containing antihypertensive agents include calcium channel blockers, such as Cardizem®, Adalat®, Calan®, Cardene®, Covera®, Dilacor®, DynaCirc®, Procardia XL®, Sular®, Tiazac®, Vascor®, Verelan®, Isoptin®, Nimotop®, Norvasc®, and Plendil®; angiotensin converting enzyme (ACE) inhibitors, such as Accupril®, Altace®, Captopril®, Lotensin®, Mavik®, Monopril®, Prinivil®, Univasc®, Vasotec® and Zestril®.

Angiotensin Converting Enzyme Inhibitors (ACE-Inhibitors) which are within the scope of this invention include, but are not limited to: alacepril, which may be prepared as disclosed in U.S. Pat. No. 4,248,883; benazepril, which may be prepared as disclosed in U.S. Pat. No. 4,410,520; captopril, which may be prepared as disclosed in U.S. Pat. Nos. 4,046,889 and 4,105,776; ceronapril, which may be prepared as disclosed in U.S. Pat. No. 4,452,790; delapril, which may be prepared as disclosed in U.S. Pat. No. 4,385,051; enalapril, which may be prepared as disclosed in U.S. Pat. No. 4,374,829; fosinopril, which may be prepared as disclosed in U.S. Pat. No. 4,337,201; imadapril, which may be prepared as disclosed in U.S. Pat. No. 4,508,727; lisinopril, which may be prepared as disclosed in U.S. Pat. No. 4,555,502; moveltopril, which may be prepared as disclosed in Belgian Patent No. 893,553; perindopril, which may be prepared as disclosed in U.S. Pat. No. 4,508,729; quinapril, which may be prepared as disclosed in U.S. Pat. No. 4,344,949; ramipril, which may be prepared as disclosed in U.S. Pat. No. 4,587,258; spirapril, which may be prepared as disclosed in U.S. Pat. No. 4,470,972; temocapril, which may be prepared as disclosed in U.S. Pat. No. 4,699,905; and trandolapril, which may be prepared as disclosed in U.S. Pat. No. 4,933,361. The disclosures of all such U.S. patents are incorporated herein by reference.

Angiotensin-II receptor antagonists (A-II antagonists) which are within the scope of this invention include, but are not limited to: candesartan, which may be prepared as disclosed in U.S. Pat. No. 5,196,444; eprosartan, which may be prepared as disclosed in U.S. Pat. No. 5,185,351; irbesartan, which may be prepared as disclosed in U.S. Pat. No. 5,270,317; losartan, which may be prepared as disclosed in U.S. Pat. No. 5,138,069; and valsartan, which may be prepared as disclosed in U.S. Pat. No. 5,399,578. The disclosures of all such U.S. patents are incorporated herein by reference.

Beta-adrenergic receptor blockers (beta- or β-blockers) which are within the scope of this invention include, but are not limited to: acebutolol, which may be prepared as disclosed in U.S. Pat. No. 3,857,952; alprenolol, which may be prepared as disclosed in Netherlands Patent Application No. 6,605,692; amosulalol, which may be prepared as disclosed in U.S. Pat. No. 4,217,305; arotinolol, which may be prepared as disclosed in U.S. Pat. No. 3,932,400; atenolol, which may be prepared as disclosed in U.S. Pat. No. 3,663,607 or 3,836,671; befunolol, which may be prepared as disclosed in U.S. Pat. No. 3,853,923; betaxolol, which may be prepared as disclosed in U.S. Pat. No. 4,252,984; bevantolol, which may be prepared as disclosed in U.S. Pat. No. 3,857,981; bisoprolol, which may be prepared as disclosed in U.S. Pat. No. 4,171,370; bopindolol, which may be prepared as disclosed in U.S. Pat. No. 4,340,541; bucumolol, which may be prepared as disclosed in U.S. Pat. No. 3,663,570; bufetolol, which may be prepared as disclosed in U.S. Pat. No. 3,723,476; bufuralol, which may be prepared as disclosed in U.S. Pat. No. 3,929,836; bunitrolol, which may be prepared as disclosed in U.S. Pat. Nos. 3,940,489 and 3,961,071; buprandolol, which may be prepared as disclosed in U.S. Pat. No. 3,309,406; butiridine hydrochloride, which may be prepared as disclosed in French Patent No. 1,390,056; butofilolol, which may be prepared as disclosed in U.S. Pat. No. 4,252,825; carazolol, which may be prepared as disclosed in German Patent No. 2,240,599; carteolol, which may be prepared as disclosed in U.S. Pat. No. 3,910,924; carvedilol, which may be prepared as disclosed in U.S. Pat. No. 4,503,067; celiprolol, which may be prepared as disclosed in U.S. Pat. No. 4,034,009; cetamolol, which may be prepared as disclosed in U.S. Pat. No. 4,059,622; cloranolol, which may be prepared as disclosed in German Patent No. 2,213,044; dilevalol, which may be prepared as disclosed in Clifton et al., Journal of Medicinal Chemistry, 1982, 25, 670; epanolol, which may be prepared as disclosed in European Patent Publication Application No. 41,491; indenolol, which may be prepared as disclosed in U.S. Pat. No. 4,045,482; labetalol, which may be prepared as disclosed in U.S. Pat. No. 4,012,444; levobunolol, which may be prepared as disclosed in U.S. Pat. No. 4,463,176; mepindolol, which may be prepared as disclosed in Seeman et al., Helv. Chim. Acta, 1971, 54, 241; metipranolol, which may be prepared as disclosed in Czechoslovakian Patent Application No. 128,471; metoprolol, which may be prepared as disclosed in U.S. Pat. No. 3,873,600; moprolol, which may be prepared as disclosed in U.S. Pat. No. 3,501,769l; nadolol, which may be prepared as disclosed in U.S. Pat. No. 3,935,267; nadoxolol, which may be prepared as disclosed in U.S. Pat. No. 3,819,702; nebivalol, which may be prepared as disclosed in U.S. Pat. No. 4,654,362; nipradilol, which may be prepared as disclosed in U.S. Pat. No. 4,394,382; oxprenolol, which may be prepared as disclosed in British Patent No. 1,077,603; perbutolol, which may be prepared as disclosed in U.S. Pat. No. 3,551,493; pindolol, which may be prepared as disclosed in Swiss Patent Nos. 469,002 and 472,404; practolol, which may be prepared as disclosed in U.S. Pat. No. 3,408,387; pronethalol, which may be prepared as disclosed in British Patent No. 909,357; propranolol, which may be prepared as disclosed in U.S. Pat. Nos. 3,337,628 and 3,520,919; sotalol, which may be prepared as disclosed in Uloth et al., Journal of Medicinal Chemistry, 1966, 9, 88; sufinalol, which may be prepared as disclosed in German Patent No. 2,728,641; talindol, which may be prepared as disclosed in U.S. Pat. Nos. 3,935,259 and 4,038,313; tertatolol, which may be prepared as disclosed in U.S. Pat. No. 3,960,891; tilisolol, which may be prepared as disclosed in U.S. Pat. No. 4,129,565; timolol, which may be prepared as disclosed in U.S. Pat. No. 3,655,663; toliprolol, which may be prepared as disclosed in U.S. Pat. No. 3,432,545; and xibenolol, which may be prepared as disclosed in U.S. Pat. No. 4,018,824. The disclosures of all such U.S. patents are incorporated herein by reference.

Alpha-adrenergic receptor blockers (alpha- or α-blockers) which are within the scope of this invention include, but are not limited to: amosulalol, which may be prepared as disclosed in U.S. Pat. No. 4,217,307; arotinolol, which may be prepared as disclosed in U.S. Pat. No. 3,932,400; dapiprazole, which may be prepared as disclosed in U.S. Pat. No. 4,252,721; doxazosin, which may be prepared as disclosed in U.S. Pat. No. 4,188,390; fenspiride, which may be prepared as disclosed in U.S. Pat. No. 3,399,192; indoramin, which may be prepared as disclosed in U.S. Pat. No. 3,527,761; labetolol, which may be prepared as disclosed above; naftopidil, which may be prepared as disclosed in U.S. Pat. No. 3,997,666; nicergoline, which may be prepared as disclosed in U.S. Pat. No. 3,228,943; prazosin, which may be prepared as disclosed in U.S. Pat. No. 3,511,836; tamsulosin, which may be prepared as disclosed in U.S. Pat. No. 4,703,063; tolazoline, which may be prepared as disclosed in U.S. Pat. No. 2,161,938; trimazosin, which may be prepared as disclosed in U.S. Pat. No. 3,669,968; and yohimbine, which may be isolated from natural sources according to methods well known to those skilled in the art. The disclosures of all such U.S. patents are incorporated herein by reference.

The term “vasodilator,” where used herein, is meant to include cerebral vasodilators, coronary vasodilators and peripheral vasodilators. Cerebral vasodilators within the scope of this invention include, but are not limited to: bencyclane, which may be prepared as disclosed above; cinnarizine, which may be prepared as disclosed above; citicoline, which may be isolated from natural sources as disclosed in Kennedy et al., Journal of the American Chemical Society, 1955, 77, 250 or synthesized as disclosed in Kennedy, Journal of Biological Chemistry, 1956, 222, 185; cyclandelate, which may be prepared as disclosed in U.S. Pat. No. 3,663,597; ciclonicate, which may be prepared as disclosed in German Patent No. 1,910,481; diisopropylamine dichloroacetate, which may be prepared as disclosed in British Patent No. 862,248; eburnamonine, which may be prepared as disclosed in Hermann et al., Journal of the American Chemical Society, 1979, 101, 1540; fasudil, which may be prepared as disclosed in U.S. Pat. No. 4,678,783; fenoxedil, which may be prepared as disclosed in U.S. Pat. No. 3,818,021; flunarizine, which may be prepared as disclosed in U.S. Pat. No. 3,773,939; ibudilast, which may be prepared as disclosed in U.S. Pat. No. 3,850,941; ifenprodil, which may be prepared as disclosed in U.S. Pat. No. 3,509,164; lomerizine, which may be prepared as disclosed in U.S. Pat. No. 4,663,325; nafronyl, which may be prepared as disclosed in U.S. Pat. No. 3,334,096; nicametate, which may be prepared as disclosed in Blicke et al., Journal of the American Chemical Society, 1942, 64, 1722; nicergoline, which may be prepared as disclosed above; nimodipine, which may be prepared as disclosed in U.S. Pat. No. 3,799,934; papaverine, which may be prepared as reviewed in Goldberg, Chem. Prod. Chem. News, 1954, 17, 371; pentifylline, which may be prepared as disclosed in German Patent No. 860,217; tinofedrine, which may be prepared as disclosed in U.S. Pat. No. 3,563,997; vincamine, which may be prepared as disclosed in U.S. Pat. No. 3,770,724; vinpocetine, which may be prepared as disclosed in U.S. Pat. No. 4,035,750; and viquidil, which may be prepared as disclosed in U.S. Pat. No. 2,500,444. The disclosures of all such U.S. patents are incorporated herein by reference.

Coronary vasodilators within the scope of this invention include, but are not limited to: amotriphene, which may be prepared as disclosed in U.S. Pat. No. 3,010,965; bendazol, which may be prepared as disclosed in J. Chem. Soc. 1958, 2426; benfurodil hemisuccinate, which may be prepared as disclosed in U.S. Pat. No. 3,355,463; benziodarone, which may be prepared as disclosed in U.S. Pat. No. 3,012,042; chloracizine, which may be prepared as disclosed in British Patent No. 740,932; chromonar, which may be prepared as disclosed in U.S. Pat. No. 3,282,938; clobenfural, which may be prepared as disclosed in British Patent No. 1,160,925; clonitrate, which may be prepared from propanediol according to methods well known to those skilled in the art, e.g., see Annalen, 1870, 155, 165; cloricromen, which may be prepared as disclosed in U.S. Pat. No. 4,452,811; dilazep, which may be prepared as disclosed in U.S. Pat. No. 3,532,685; dipyridamole, which may be prepared as disclosed in British Patent No. 807,826; droprenilamine, which may be prepared as disclosed in German Patent No. 2,521,113; efloxate, which may be prepared as disclosed in British Patent Nos. 803,372 and 824,547; erythrityl tetranitrate, which may be prepared by nitration of erythritol according to methods well-known to those skilled in the art; etafenone, which may be prepared as disclosed in German Patent No. 1,265,758; fendiline, which may be prepared as disclosed in U.S. Pat. No. 3,262,977; floredil, which may be prepared as disclosed in German Patent No. 2,020,464; ganglefene, which may be prepared as disclosed in U.S.S.R. Patent No. 115,905; hexestrol, which may be prepared as disclosed in U.S. Pat. No. 2,357,985; hexobendine, which may be prepared as disclosed in U.S. Pat. No. 3,267,103; itramin tosylate, which may be prepared as disclosed in Swedish Patent No. 168,308; khellin, which may be prepared as disclosed in Baxter et al., Journal of the Chemical Society, 1949, S 30; lidoflazine, which may be prepared as disclosed in U.S. Pat. No. 3,267,104; mannitol hexanitrate, which may be prepared by the nitration of mannitol according to methods well-known to those skilled in the art; medibazine, which may be prepared as disclosed in U.S. Pat. No. 3,119,826; nitroglycerin; pentaerythritol tetranitrate, which may be prepared by the nitration of pentaerythritol according to methods well-known to those skilled in the art; pentrinitrol, which may be prepared as disclosed in German Patent No. 638,422-3; perhexilline, which may be prepared as disclosed above; pimefylline, which may be prepared as disclosed in U.S. Pat. No. 3,350,400; prenylamine, which may be prepared as disclosed in U.S. Pat. No. 3,152,173; propatyl nitrate, which may be prepared as disclosed in French Patent No. 1,103,113; trapidil, which may be prepared as disclosed in East German Patent No. 55,956; tricromyl, which may be prepared as disclosed in U.S. Pat. No. 2,769,015; trimetazidine, which may be prepared as disclosed in U.S. Pat. No. 3,262,852; trolnitrate phosphate, which may be prepared by nitration of triethanolamine followed by precipitation with phosphoric acid according to methods well-known to those skilled in the art; visnadine, which may be prepared as disclosed in U.S. Pat. Nos. 2,816,118 and 2,980,699. The disclosures of all such U.S. patents are incorporated herein by reference.

Peripheral vasodilators within the scope of this invention include, but are not limited to: aluminum nicotinate, which may be prepared as disclosed in U.S. Pat. No. 2,970,082; bamethan, which may be prepared as disclosed in Corrigan et al., Journal of the American Chemical Society, 1945, 67, 1894; bencyclane, which may be prepared as disclosed above; betahistine, which may be prepared as disclosed in Walter et al.; Journal of the American Chemical Society, 1941, 63, 2771; bradykinin, which may be prepared as disclosed in Hamburg et al., Arch. Biochem. Biophys., 1958, 76, 252; brovincamine, which may be prepared as disclosed in U.S. Pat. No. 4,146,643; bufeniode, which may be prepared as disclosed in U.S. Pat. No. 3,542,870; buflomedil, which may be prepared as disclosed in U.S. Pat. No. 3,895,030; butalamine, which may be prepared as disclosed in U.S. Pat. No. 3,338,899; cetiedil, which may be prepared as disclosed in French Patent Nos. 1,460,571; ciclonicate, which may be prepared as disclosed in German Patent No. 1,910,481; cinepazide, which may be prepared as disclosed in Belgian Patent No. 730,345; cinnarizine, which may be prepared as disclosed above; cyclandelate, which may be prepared as disclosed above; diisopropylamine dichloroacetate, which may be prepared as disclosed above; eledoisin, which may be prepared as disclosed in British Patent No. 984,810; fenoxedil, which may be prepared as disclosed above; flunarizine, which may be prepared as disclosed above; hepronicate, which may be prepared as disclosed in U.S. Pat. No. 3,384,642; ifenprodil, which may be prepared as disclosed above; iloprost, which may be prepared as disclosed in U.S. Pat. No. 4,692,464; inositol niacinate, which may be prepared as disclosed in Badgett et al., Journal of the American Chemical Society, 1947, 69, 2907; isoxsuprine, which may be prepared as disclosed in U.S. Pat. No. 3,056,836; kallidin, which may be prepared as disclosed in Biochem. Biophys. Res. Commun., 1961, 6, 210; kallikrein, which may be prepared as disclosed in German Patent No. 1,102,973; moxisylyte, which may be prepared as disclosed in German Patent No. 905,738; nafronyl, which may be prepared as disclosed above; nicametate, which may be prepared as disclosed above; nicergoline, which may be prepared as disclosed above; nicofuranose, which may be prepared as disclosed in Swiss Patent No. 366,523; nylidrin, which may be prepared as disclosed in U.S. Pat. Nos. 2,661,372 and 2,661,373; pentifylline, which may be prepared as disclosed above; pentoxifylline, which may be prepared as disclosed in U.S. Pat. No. 3,422,107; piribedil, which may be prepared as disclosed in U.S. Pat. No. 3,299,067; prostaglandin E₁, which may be prepared by any of the methods referenced in the Merck Index, Twelfth Edition, Budaveri, Ed., New Jersey, 1996, p. 1353; suloctidil, which may be prepared as disclosed in German Patent No. 2,334,404; tolazoline, which may be prepared as disclosed in U.S. Pat. No. 2,161,938; and xanthinol niacinate, which may be prepared as disclosed in German Patent No. 1,102,750 or Korbonits et al., Acta. Pharm. Hung., 1968, 38, 98. The disclosures of all such U.S. patents are incorporated herein by reference.

The term “diuretic,” within the scope of this invention, is meant to include diuretic benzothiadiazine derivatives, diuretic organomercurials, diuretic purines, diuretic steroids, diuretic sulfonamide derivatives, diuretic uracils and other diuretics such as amanozine, which may be prepared as disclosed in Austrian Patent No. 168,063; amiloride, which may be prepared as disclosed in Belgian Patent No. 639,386; arbutin, which may be prepared as disclosed in Tschitschibabin, Annalen, 1930, 479, 303; chlorazanil, which may be prepared as disclosed in Austrian Patent No. 168,063; ethacrynic acid, which may be prepared as disclosed in U.S. Pat. No. 3,255,241; etozolin, which may be prepared as disclosed in U.S. Pat. No. 3,072,653; hydracarbazine, which may be prepared as disclosed in British Patent No. 856,409; isosorbide, which may be prepared as disclosed in U.S. Pat. No. 3,160,641; mannitol; metochalcone, which may be prepared as disclosed in Freudenberg et al., Ber., 1957, 90, 957; muzolimine, which may be prepared as disclosed in U.S. Pat. No. 4,018,890; perhexiline, which may be prepared as disclosed above; ticrynafen, which may be prepared as disclosed in U.S. Pat. No. 3,758,506; triamterene which may be prepared as disclosed in U.S. Pat. No. 3,081,230; and urea. The disclosures of all such U.S. patents are incorporated herein by reference.

Diuretic benzothiadiazine derivatives within the scope of this invention include, but are not limited to: althiazide, which may be prepared as disclosed in British Patent No. 902,658; bendroflumethiazide, which may be prepared as disclosed in U.S. Pat. No. 3,265,573; benzthiazide, McManus et al., 136th Am. Soc. Meeting (Atlantic City, September 1959), Abstract of papers, pp 13-0; benzylhydrochlorothiazide, which may be prepared as disclosed in U.S. Pat. No. 3,108,097; buthiazide, which may be prepared as disclosed in British Patent Nos. 861,367 and 885,078; chlorothiazide, which may be prepared as disclosed in U.S. Pat. Nos. 2,809,194 and 2,937,169; chlorthalidone, which may be prepared as disclosed in U.S. Pat. No. 3,055,904; cyclopenthiazide, which may be prepared as disclosed in Belgian Patent No. 587,225; cyclothiazide, which may be prepared as disclosed in Whitehead et al., Journal of Organic Chemistry, 1961, 26, 2814; epithiazide, which may be prepared as disclosed in U.S. Pat. No. 3,009,911; ethiazide, which may be prepared as disclosed in British Patent No. 861,367; fenquizone, which may be prepared as disclosed in U.S. Pat. No. 3,870,720; indapamide, which may be prepared as disclosed in U.S. Pat. No. 3,565,911; hydrochlorothiazide, which may be prepared as disclosed in U.S. Pat. No. 3,164,588; hydroflumethiazide, which may be prepared as disclosed in U.S. Pat. No. 3,254,076; methyclothiazide, which may be prepared as disclosed in Close et al., Journal of the American Chemical Society, 1960, 82, 1132; meticrane, which may be prepared as disclosed in French Patent Nos. M2790 and 1,365,504; metolazone, which may be prepared as disclosed in U.S. Pat. No. 3,360,518; paraflutizide, which may be prepared as disclosed in Belgian Patent No. 620,829; polythiazide, which may be prepared as disclosed in U.S. Pat. No. 3,009,911; quinethazone, which may be prepared as disclosed in U.S. Pat. No. 2,976,289; teclothiazide, which may be prepared as disclosed in Close et al., Journal of the American Chemical Society, 1960, 82, 1132; and trichlormethiazide, which may be prepared as disclosed in deStevens et al., Experientia, 1960, 16, 113. The disclosures of all such U.S. patents are incorporated herein by reference.

Diuretic sulfonamide derivatives within the scope of this invention include, but are not limited to: acetazolamide, which may be prepared as disclosed in U.S. Pat. No. 2,980,679; ambuside, which may be prepared as disclosed in U.S. Pat. No. 3,188,329; azosemide, which may be prepared as disclosed in U.S. Pat. No. 3,665,002; bumetanide, which may be prepared as disclosed in U.S. Pat. No. 3,634,583; butazolamide, which may be prepared as disclosed in British Patent No. 769,757; chloraminophenamide, which may be prepared as disclosed in U.S. Pat. Nos. 2,809,194, 2,965,655 and 2,965,656; clofenamide, which may be prepared as disclosed in Olivier, Rec. Tray. Chim., 1918, 37, 307; clopamide, which may be prepared as disclosed in U.S. Pat. No. 3,459,756; clorexolone, which may be prepared as disclosed in U.S. Pat. No. 3,183,243; disulfamide, which may be prepared as disclosed in British Patent No. 851,287; ethoxolamide, which may be prepared as disclosed in British Patent No. 795,174; furosemide, which may be prepared as disclosed in U.S. Pat. No. 3,058,882; mefruside, which may be prepared as disclosed in U.S. Pat. No. 3,356,692; methazolamide, which may be prepared as disclosed in U.S. Pat. No. 2,783,241; piretanide, which may be prepared as disclosed in U.S. Pat. No. 4,010,273; torasemide, which may be prepared as disclosed in U.S. Pat. No. 4,018,929; tripamide, which may be prepared as disclosed in Japanese Patent No. 73 05,585; and xipamide, which may be prepared as disclosed in U.S. Pat. No. 3,567,777. The disclosures of all such U.S. patents are incorporated herein by reference.

The starting materials and reagents for the above-described compounds of the present invention and combination agents are also readily available or can be easily synthesized by those skilled in the art using conventional methods of organic synthesis. For example, many of the compounds used herein, are related to, or are derived from compounds in which there is a large scientific interest and commercial need, and accordingly many such compounds are commercially available or are reported in the literature or are easily prepared from other commonly available substances by methods which are reported in the literature.

Some of the compounds or combination agents of the present invention or intermediates in their synthesis have asymmetric carbon atoms and therefore are enantiomers or diastereomers. Diasteromeric mixtures can be separated into their individual diastereomers on the basis of their physical chemical differences by methods known per se, for example, by chromatography and/or fractional crystallization. Enantiomers can be separated by, for example, chiral HPLC methods or converting the enantiomeric mixture into a diasteromeric mixture by reaction with an appropriate optically active compound (e.g., alcohol), separating the diastereomers and converting (e.g., hydrolyzing) the individual diastereomers to the corresponding pure enantiomers. Also, an enantiomeric mixture of the compounds or an intermediate in their synthesis which contain an acidic or basic moiety may be separated into their compounding pure enantiomers by forming a diastereomeric salt with an optically pure chiral base or acid (e.g., 1-phenyl-ethyl amine or tartaric acid) and separating the diasteromers by fractional crystallization followed by neutralization to break the salt, thus providing the corresponding pure enantiomers. All such isomers, including diastereomers, enantiomers and mixtures thereof are considered as part of the present invention. Also, some of the compounds of the present invention are atropisomers (e.g., substituted biaryls) and are considered as part of the present invention.

More specifically, the compounds or combination agents of the present invention can be obtained by fractional crystallization of the basic intermediate with an optically pure chiral acid to form a diastereomeric salt. Neutralization techniques are used to remove the salt and provide the enantiomerically pure compounds. Alternatively, the compounds of the present invention may be obtained in enantiomerically enriched form by resolving the racemate of the final compound or an intermediate in its synthesis (preferably the final compound) employing chromatography (preferably high pressure liquid chromatography [HPLC]) on an asymmetric resin (preferably Chiralcel™ AD or OD (obtained from Chiral Technologies, Exton, Pa.)) with a mobile phase consisting of a hydrocarbon (preferably heptane or hexane) containing between 0 and 50% isopropanol (preferably between 2 and 20%) and between 0 and 5% of an alkyl amine (preferably 0.1% of diethylamine). Concentration of the product containing fractions affords the desired materials.

Some of the compounds of this invention or combination agents are basic or zwitterionic and form salts with pharmaceutically acceptable anions. All such salts are within the scope of this invention and they can be prepared by conventional methods such as combining the acidic and basic entities, usually in a stoichiometric ratio, in either an aqueous, non-aqueous or partially aqueous medium, as appropriate. The salts are recovered either by filtration, by precipitation with a non-solvent followed by filtration, by evaporation of the solvent, or, in the case of aqueous solutions, by lyophilization, as appropriate. The compounds are obtained in crystalline form according to procedures known in the art, such as by dissolution in an appropriate solvent(s) such as ethanol, hexanes or water/ethanol mixtures.

Some of the combination agents of the present invention are acidic and they form a salt with a pharmaceutically acceptable cation. All such salts are within the scope of the present invention and they can be prepared by conventional methods such as combining the acidic and basic entities, usually in a stoichiometric ratio, in either an aqueous, non-aqueous or partially aqueous medium, as appropriate. The salts are recovered either by filtration, by precipitation with a non-solvent followed by filtration, by evaporation of the solvent, or, in the case of aqueous solutions, by lyophilization, as appropriate. The compounds can be obtained in crystalline form by dissolution in an appropriate solvent(s) such as ethanol, hexanes or water/ethanol mixtures.

Certain compounds of the present invention or combination agents may exist in more than one crystal form (generally referred to as “polymorphs”). Polymorphs may be prepared by crystallization under various conditions, for example, using different solvents or different solvent mixtures for recrystallization; crystallization at different temperatures; and/or various modes of cooling, ranging from very fast to very slow cooling during crystallization. Polymorphs may also be obtained by heating or melting the compound of the present invention followed by gradual or fast cooling. The presence of polymorphs may be determined by solid probe NMR spectroscopy, IR spectroscopy, differential scanning calorimetry, powder X-ray diffraction or such other techniques.

Isotopically-labelled compounds of Formula I or combination agents can generally be prepared by conventional techniques known to those skilled in the art or by processes analogous to those described in the accompanying Examples and Preparations using an appropriate isotopically-labelled reagents in place of the non-labelled reagent previously employed.

Proprotein convertase subtilisin/kexin type 9, also known as PCSK9, is an enzyme that in humans is encoded by the PCSK9 gene. As defined herein, and typically known to those skilled in the art, the definition of PCSK9 also includes greater than 50 gain and loss of function mutations, GOF and LOF, respectively, thereof.

(http://www.ucl.ac.uk/ldlr/LOVDv.1.1.0/search.php?select_db=PCSK9&srch=all).

The compounds of this invention preferably inhibit the translation of PCSK9 mRNA to PCSK9 protein.

As defined herein inhibition of translation of PCSK9 mRNA to PCSK9 protein is determined by the “Cell Free PCSK9 Assay” provided herein in the specification. This “Cell Free PCSK9 Assay” is specific to the production of PCSK9 protein from PCSK9 mRNA and therefore detects inhibitors of this translational process rather than other mechanisms by which PCSK9 protein may be reduced. Any compound (whose active moiety or compound itself) that presents an IC₅₀ (μM) below about 50 μM in the “Cell Free PCSK9 Assay” is considered as inhibiting PCSK9 translation. It is preferred that the 10₅₀ of the compound is less than about 30 μM and especially preferred that the IC₅₀ of the compound is less than about 20 μM.

It is preferred that the compound “selectively” inhibits translation of PCSK9 mRNA to PCSK9 protein. The term “selective” is defined as “inhibiting” translation of less than 1 percentage of proteins in a typical global proteomic assay. It is preferred that the level is below about 0.5% of proteins and especially preferred that the level is below about 0.1% of proteins. Typically in a standard assay the 1% level equates to about 40 non-PCSK9 proteins out of about 4000 proteins.

Inhibition of the target protein is defined as percent translational reduction of the target protein, in increasing preference in the order given, of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99%—in relation to translation of the target protein in a control cell not exposed to the agent. The amount of translational reduction needed in connection with treating a condition may depend upon whether additional types of agents are being co-administered with the agents of the invention for treatment of the condition. This definition of “inhibition” related to the Global Proteomic Assay is not to be confused with the previous definition of “inhibition” related to the Cell Free PCSK9 Assay.

Selectivity of the agent for inhibiting the target gene in relation to the total measurable proteome can be assessed using ribosomal foot printing or ribosome profiling techniques known in the art, such as those disclosed in U.S. Pat. No. 8,486,865 to Weissman et al, the disclosure of which is incorporated by reference. The abundance of protected RNA can be correlated to the rate of translation of the RNA or the relative rate of translation compared to other RNAs. The nucleic acid amplification and sequencing methodology (including “deep sequencing”) associated with these techniques are known to those skilled in the art.

The compounds of the present invention, their prodrugs and the salts of such compounds and prodrugs are all adapted to therapeutic use as agents that antagonize extracellular proprotein convertase subtilisin kexin type 9 (PCSK9) activity, including its interaction with the low density lipoprotein (LDL) receptor (LDLR), in mammals, particularly humans. Thus, it is believed as has been demonstrated in human individuals with loss of function (LOF) PCSK9 mutations (e.g. Hobbs et. al. NEJM, 2006 and Hobbs et. al. Am. J. Hum. Gen., 2006) the compounds of the present invention, by decreasing PCSK9 levels, increase the cell surface expression of the LDL receptor and accordingly reduce LDL cholesterol. Hence, these compounds are useful for the treatment and correction of the various dyslipidemias observed to be associated with the development and incidence of atherosclerosis and cardiovascular disease, including hypoalphalipoproteinemia and hypertriglyceridemia.

Given the positive correlation between LDL cholesterol, and their associated apolipoproteins in blood with the development of cardiovascular, cerebral vascular and peripheral vascular diseases, the compounds of the present invention and the salts of such compounds, by virtue of their pharmacologic action, are useful for the prevention, arrestment and/or regression of atherosclerosis and its associated disease states. These include cardiovascular disorders (e.g., coronary artery disease, cerebrovascular disease, coronary artery disease, ventricular dysfunction, cardiac arrhythmia, pulmonary vascular disease, vascular hemostatic disease, cardiac ischemia and myocardial infarction), complications due to cardiovascular disease, transient cerebral ischemic attacks).

The utility of the compounds of the present invention and the salts of such compounds as medical agents in the treatment of the above described disease/conditions in mammals (e.g. humans, male or female) is demonstrated by the activity of the compounds of the present invention in one or more of the conventional assays and in vivo assays described below. The in vivo assays (with appropriate modifications within the skill in the art) can be used to determine the activity of other lipid or triglyceride controlling agents as well as the compounds of the present invention. Thus, the protocols described below can also be used to demonstrate the utility of the combinations of the agents (i.e., the compounds of the present invention) described herein. In addition, such assays provide a means whereby the activities of the compounds of the present invention and the salts of such compounds (or the other agents described herein) can be compared to each other and with the activities of other known compounds. The results of these comparisons are useful for determining dosage levels in mammals, including humans, for the treatment of such diseases. The following protocols can of course be varied by those skilled in the art.

In particular, the human intestinal S9 fraction in vitro stability assay (H_{Int}) and human hepatocyte in vitro liver metabolism assay (H_{Hep}) provide important information regarding the clearance and metabolic activation of these compounds. The human intestinal S9 fraction in vitro stability assay provides a surrogate measure of compound metabolism as it travels across the gut wall; compounds with low CLint values more likely to enter the portal vein and be exposed to the liver. Likewise, the human hepatocyte in vitro liver metabolism assay provides a surrogate measure of compound metabolism when exposed to liver; compounds with high CLᵢₙₜ values are more likely to be metabolically activated. For compounds such as prodrugs that release an active species on metabolic activation, high CLᵢₙₜ values in human hepatocytes are desirable. Active compounds released in this way inhibit PCSK9 and show improved atherosclerotic properties by increased exposure of the active metabolite in the liver. These data are shown in Table I.

### Human Intestinal S9 Fraction In Vitro Stability Assay (Hᵢₙₜ)

In vitro stability of test compounds in human intestinal S9 fraction was determined by a substrate depletion approach. Frozen PMSF-free human intestinal S9 (BD Gentest) was thawed on wet ice and diluted to the test concentration of 0.1 mg/mL in 100 mM potassium phosphate buffer pH 7.4. Aliquots of diluted intestinal S9 (495 μL, n=2) were added to tubes in a dry heat bath and pre-warmed for 5 min at 37° C. Test compounds were dissolved in DMSO at 30 mM, ordered from the TekCel at 10 mM, and further diluted to 0.1 mM in DMSO. To initiate the reaction, 5 μL of 0.1 mM DMSO stock solution was added to the pre-warmed intestinal S9. The final test compound concentration in the incubation was 1 μM. At each time point (0.25, 5, 10, 20, 40, and 60 min) a 50 μL sample of incubate was removed and transferred to a plate containing 200 μL acetonitrile with internal standard (2 ng/mL terfenadine). After collection of the final time point, sample plates were capped, vortexed, and centrifuged for 5 minutes at approximately 2000×g. 150 μL of supernatant was removed and transferred to a clean storage plate for direct LC-MS/MS analysis. LC-MS/MS analysis was conducted on a Triple Quad 5500 (AB Sciex) with two LC-20AD pumps and CBM-20 controller (Shimadzu) and CTC PAL autosampler (LEAP Technologies). The MS was operated in multiple reaction monitoring mode with simultaneous monitoring for test compound and internal standard. 5 μL of sample was injected on a Kinetex C18 30×2.1 mm column (Phenomenex) and eluted at 0.5 ml/min under the following conditions, where solvent A was water containing 0.1% formic acid and solvent B was acetonitrile containing 0.1% formic acid: hold initial conditions 90% A and 10% B for 0.8 min, ramp to 30% A and 70% B over 1 min, step to 5% A and 95% B over 0.05 min, hold at 5% A and 95% B for 0.15 min, return to initial conditions over 0.1 min, and hold for 0.4 min. Peak areas of test compound and internal standard were quantitated using Analyst 1.5 (AB Sciex) and the ratios of test compound peak area to internal standard peak area (area ratio) were calculated. The natural log of area ratio was plotted versus time and the portion of the curve representing the initial linear rate of test compound depletion was fit using linear regression (IDBS E-Workbook 9.4). The slope of this line was converted to half-life (t_{1/2}=−LN2/slope). Half-life was used to calculate intrinsic apparent clearance (CLint=LN2/(t_{1/2}*(mg protein/ml incubation))).

### Human Hepatocyte In Vitro Liver Metabolism Assay (H_{Hep})

In order to determine the rate of metabolism leading to conversion of prodrug into active drug form, experiments utilizing human hepatocytes were performed. Hepatocytes are an ideal in vitro system to monitor hepatic metabolism since these intact cells contain all the hepatic enzymes found in vivo, including phase I enzymes (such as CYPs, aldehyde oxidases, esterases and MAOs) and phase II enzymes (such as UDP-glucuronyltransferases and sulfotransferases). The assay utilizes isolated hepatocytes from human donors incubated with the compound of interest in conditions mimicking physiological conditions where the metabolic stability of the compound is investigated. The experimental protocol is as follows. Vials of cryopreserved human hepatocytes (stored in liquid nitrogen until used for testing) were thawed in a water bath (37 to 40° C.) until nearly thawed, transferred to a conical tube, resuspended by inversion and subsequently centrifuged at 50-90 g at room temperature for 5 min. The supernatant was then discarded and the pellet loosened by gently tapping the end of the conical tube. William's E media was then added to achieve the desired final cell density (0.5 million viable cells per mL), and the hepatocytes were then resuspended in this fresh media. The viable cell count was then determined using the trypan blue exclusion method where a minimum viability of 70% was obtained. At this point, new molecular entities (NME's) were prepared for testing. In brief, the NME was diluted with DMSO such that final incubation concentration of NME was 1 and final DMSO content was 0.1%. Assays were conducted in a 384-well format at 37° C. in an incubator held at 95% air to 5% CO₂ at 95% relative humidity. The per-well incubation total incubation volume was 20 μL including hepatocytes and NME. The assay was performed using 7 hepatocyte plates where the plates were designated as sampling times 0, 15, 30, 60, 120 and 240 min and include hepatocytes and NME, and a no NME control plate with hepatocytes that was taken at 240 min. Two additional no hepatocyte containing control plates were prepared and subsequently sampled at 0 and 240 min, respectively, and were identical to the hepatocyte containing plates with respect to NME and media composition. The incubations were stopped using acetonitrile and prepared for analytical testing using liquid chromatography mass-spectrometry (LC/MS) detection. Each NME was optimized for LC/MS analytical conditions. A disappearance curve was generated from the sample time point analytical peak areas and compared to control plate results (control plates allow artifacts such as non-hepatocyte mediated decline (e.g., media/condition instability for the NME) to be determined). The slope of the disappearance curve was used to determine metabolic stability expressed CLᵢₙₜ. Performance of the assay with regards to expected metabolic activity was monitored in separate well using positive controls including propranolol, midazolam and naloxone (each probes for specific enzymatic activity).

### PCSK9 AlphaLISA Assay

### PCSK9 Lowering in WT7 Cells

An in-vitro AlphaLISA assay (Perkin Elmer) was developed in order to quantitate the level of PCSK9 secreted into the cell culture media following compound treatment. To detect and measure PCSK9 protein a mouse monoclonal anti-human PCSK9 antibody was coupled to AlphaLISA acceptor beads by an external vendor (Perkin Elmer) and a second rabbit monoclonal anti-human PCSK9 antibody with an epitope distinct from that of the acceptor beads was biotinylated using the EZ link NHS-LC-LC-Biotin kit (Life Technologies #21338). Streptavidin coated-donor beads (Perkin Elmer) are also included in the assay mixture which then binds the biotinylated anti-PCSK9 antibody and in the presence of PCSK9 this donor complex and acceptor beads are brought into close proximity. Upon excitation of the donor beads at 680 nm singlet oxygen molecules are released that trigger an energy transfer cascade within the acceptor beads resolving as a single peak of light emitted at 615 nm. The ability of compound to modulate PCSK9 protein levels in conditioned media by AlphaLISA was assessed in the human hepatocellular carcinoma cell line Huh7, stably over-expressing human PCSK9. This cell line, termed WT7, was established by transfecting Huh7 cells with an in-house modified pcDNA 3.1 (+) Zeo expression vector (Life Technologies) containing the full-length human PCSK9 sequence (NCBI reference identifier, NM_174936.3, where coding sequence start annotated at position 363) and a c-terminal V5 and 6×-His tag. Following plasmid transfection the stable WT7 clone was identified and maintained under Zeocin selection. Compound screening was performed in 384-well plates where WT7 cells were plated at a density of 7500 cells per well in 20 μL of tissue culture media containing compound in an eleven point, 0.5 log dilution format at a high treatment concentration of 20 μM in a final volume of 0.5% DMSO. In additional to these test compound conditions each screening plate also included wells that contained 20 μM puromycin as a positive assay control defined as high percent effect, HPE, as well as wells containing media in 0.5% DMSO as a negative treatment control defined as zero percent effect, ZPE. After overnight compound incubation (16-24 h) the tissue culture media was collected and an aliquot from each sample was transferred to individual wells of a 384-well white Optiplate (Perkin Elmer). The coupled antibodies and donor beads were added to the assay plates in a buffer composed of 30 mM Tris pH 7.4, 0.02% Tween-20 and 0.02% Casein. Anti-PCSK9 acceptor beads (final concentration of 10 μg/mL) and anti-PCSK9 biotinylated antibody (final concentration of 3 nM) were added and incubated for 30 minutes at room temperature followed by the addition of the streptavidin donor beads (final concentration 40 μg/mL) for an additional 60 minutes. Additionally a standard curve was generated where AlphaLISA reagents were incubated in wells spiked with recombinant human PCSK9 diluted in tissue culture media from 5000 ng/mL to 0.6 ng/mL. Following incubation with AlphaLISA reagents plates were read on an EnVision (Perkin Elmer) plate reader at an excitation wavelength of 615 nM and emission/detection wavelength of 610 nM. To determine compound IC₅₀ the data for HPEand ZPEcontrol wells were first analyzed and the mean, standard deviation and Z prime calculated for each plate. The test compound data were converted into percent effect, using the ZPE and HPE controls as 0% and 100% activity, respectively. The equation used for converting each well reading into percent effect was:

### Equation 1:

$$\frac{( Test well activity value - ZPE activity value )}{( HPE activity value - ZPE activity value )} \times 100$$

IC₅₀ was then calculated and reported as the midpoint in the percent effect curve in molar units and the values are reported under the Cell Based PCSK9 IC₅₀ (μM) column header within Table 2 Biological Data. Additionally, to monitor the selectivity of compound response for PCSK9 the level of a second secreted protein, Transferrin, was measured from the same conditioned media treated with test compound by AlphaLISA. The anti-Transferrin AlphaLISA bead conjugated by PerkinElmer is a mouse monoclonal IgG1 to human transferrin (clone M10021521; cat #10-T34C; Fitzgerald). The biotinylated labeled antibody is an affinity purified goat anti-human polyclonal antibody (Cat #A80-128A; Bethyl Laboratories). To detect and quantify effects on Transferrin 0.01 mL of the culture media was transferred to a 384-well white Optiplate and 0.01 mL of media was added to bring the volume to 0.02 mL. Anti-Transferrin acceptor beads were added to a final concentration of 10 μg/mL, biotinylated anti-Transferrin at 3 nM and streptavidin donor beads at 40 μg/mL. Percent effect and IC₅₀ for Transferrin was computed in a similar manner as that described for PCSK9.

In order to eliminate the permeability barrier inherent to the WT7 cell-based assays a cell-free system was also established to assess compound activitiy. A sequence containing the full length human PCSK9 (NCBI reference identifier, NM_174936.3, where coding sequence start annotated at position 363) along with 84 additional 3′ nucleotides, comprising a V5 tag and polylinker followed by an in frame modified firefly luciferase reporter (corresponding to nucleotide positions 283-1929 of pGL3, GenBank reference identifier JN542721.1) was cloned into the pT7CFE1 expression vector (ThermoScientific). The construct was then in-vitro transcribed using the MEGAscript T7 Kit (Life Technologies) and RNA subsequently purified incorporating the MEGAclear Kit (Life Technologies) according to manufacturer's protocols. HeLa cell lysates were prepared following the protocols described by Mikami (reference is Cell-Free Protein Synthesis Systems with Extracts from Cultured Human Cells, S. Mikami, T. Kobayashi and H. Imataka; from Methods in Molecular Biology, vol. 607, pages 43-52, Y. Endo et al. (eds.), Humana Press, 2010) with the following modifications. Cells were grown in a 20 L volume of CD293 medium (Gibco 11765-054) with Glutamax increased to 4 mM, penicillin at 100 U/mL and other additions as previously described by Mikami. Growth was in a 50 L wavebag at a rocker speed of 25 rpm and angle 6.1 with 5% CO₂ and 0.2 LPM flow rate with cells harvested at a density of 2-2.5e6/mL. Lysates additionally contained 1 tablet of Roche cOmplete-EDTA protease inhibitors per 50 mL with tris(2-carboxyethyl)phosphine (Biovectra) substituted for dithiothreitol, and were clarified by an additional final centrifugation at 10,000 rpm in a Sorvall SS34 rotor at 4° C. for 10 minutes. Compound screening was performed in 384-well plates in an eleven point, 0.5 log dilution format at a top test compound concentration of 100 μM in a final volume of 0.5% DMSO. In additional to these test compound conditions each screening plate also included wells that contained 100 μM of compound example 16 (as depicted in WO2014170786; N-(3-chloropyridin-2-yl)-N-[(3R)-piperidin-3-yl]-4-(3H-[1,2,3]triazolo[4,5-b]pyridin-3-yl)benzamide) as a positive assay control defined as high percent effect, HPE, as well as wells containing media in 0.5% DMSO as a negative treatment control defined as zero percent effect, ZPE. Compounds were incubated at 30° C. for 45 minutes in a solution containing 0.1 μg of purified, in-vitro transcribed RNA together with the cell-free reaction mixture (consisting of 1.6 mM Mg and 112 mM K salts, 4.6 mM tris(2-carboxyethyl)phosphine (Biovectra), 5.0 μL HeLa lysate, 0.2 μL RNAsin (Promega) and 1.0 μL energy mix (containing 1.25 mM ATP (Sigma), 0.12 mM GTP (Sigma), 20 mM creatine phosphate (Santa Cruz), 60 μg/mL creatine phosphokinase (Sigma), 90 μg/mL tRNA (Sigma) and the 20 amino acids (Life Technologies) at final concentrations described by Mikami) and brought up in water to a final volume of 10 μL in water. Upon assay completion 1 μL from each reaction solution was removed and transferred to a second 384-well Optiplate (Perkin Elmer) containing 24 μL of SteadyGlo (Promega) and signal intensity was measured on the Envision (Perkin Elmer) using the enhanced luminescence protocol. To determine compound IC₅₀ the data for HPE and ZPE control wells were first analyzed and the mean, standard deviation and Z prime calculated for each plate. The test compound data were converted into percent effect, using the ZPE and HPE controls as 0% and 100% activity, respectively, applying Equation 1 above. IC₅₀ was then calculated and reported as the midpoint in the percent effect curve in molar units and the values are reported under the Cell Free PCSK9 IC₅₀ (μM) column header within Table 2 Biological Data.

### PCSK9 Lowering and Compound Concentration Determination in Sandwich Culture Human Hepatocytes (SCHH)

Test compound in-vitro pharmacokinetic and pharmacodynamic relationships were measured in sandwich culture primary cryopreserved human hepatocytes. Within these studies SCHH cells (BD Biosciences IVT) were thawed at 37° C. then placed on ice, after which the cells were added to pre-warmed (37° C.) In VitroGRO-HT media and centrifuged at 50×g for 3 min. The cell pellet was re-suspended to 0.8×10⁶ cells/mL in InVitroGRO-CP plating medium and cell viability determined by trypan blue exclusion. On day 1, hepatocyte suspensions were plated in BioCoat 96-well plates at a density of 80000 cells/well in a volume of 0.1 mL/well. After 18 to 24 hours of incubation at 37° C. in 5% CO₂, cells were overlaid with ice-cold 0.25 mg/mL BD Matrigel Matrix Phenol Red-Free in incubation medium at 0.1 mL/well. Cultures were maintained at 37° C. in 5% CO₂ in InVitroGRO-HI (FBS-free media), which was replaced every 24 hours and time course treatments were initiated on day 5. Prior to compound treatment cell plates were washed 3 times with 0.1 mL/well InVitroGRO-HI and 0.09 mL of media was added back in preparation for the compound additions. 1 μL of either DMSO or compound DMSO stocks at 30 mM, 10 mM, 3 mM and 1 mM were stamped into 96 well V bottom polypropylene plates. 0.099 mL of media was added to the compound plate and mixed thoroughly before the addition of 0.010 mL from the interim compound plate to the cell plate. This resulted in a final concentration of 0.1% DMSO where compounds were evaluated at 30 μM, 10 μM, 3 μM and 1 μM (in some instances compound concentrations were increased to 300 μM). Cells were incubated with compound for 5, 15, 30, 60, 180, 360, 480 and 1440 minutes at 37° C. in 5% CO₂. At the indicated time, 0.08 mL of media was removed from the cell plates and frozen for subsequent analysis of secreted PCSK9 by AlphaLISA and for determination of drug levels in the media by liquid chromatography-tandem mass spectrometry (LC-MS/MS). The remaining media was then aspirated and the cell layers were washed 3× with ice cold Hanks Balanced Salt Solution (HBSS) under shaking conditions to remove the matrigel overlay and plates were then stored at −20° C. for subsequent determination of drug levels in the cells by LC-MS/MS. AlphaLISA determination of PCSK9 protein levels within the conditioned media was performed utilizing the identical reagents and detection protocols described above for the WT7 cells. Percent PCSK9 lowering versus vehicle treated cells was then determined for each time point and the maximum response (and the corresponding concentration and time when observed) is reported under the Sandwich Culture Hepatocyte (SCHH) PCSK9 lowering summarized in Table 3.

Media samples used for test compound level determination were processed by adding 20 μL of the conditioned media to 180 μL of MeOH-IS solution or 20 μL of media matrix containing known concentrations of analyte (0-5 μM) to 180 μL of MeOH-IS. Samples were then dried under a stream of nitrogen and re-suspended in 200 μL of 50/50 MeOH/H₂O. LC-MS/MS analyses were conducted on an API-4000 triple quadrupole mass spectrometer with an atmospheric pressure electrospray ionization source (MDS SCIEX, Concord, Ontario, Canada) coupled to two Shimadzu LC-20AD pumps with a CBM-20A controller. A 10 μL sample was injected onto a Kinetex C18 column (2.6 μm, 100 Å, 30×2.1 mm, Phenomenex, Torrance, Calif.) and eluted by a mobile phase at a flow rate of 0.5 mL/min with initial conditions of 10% solvent B for 0.2 min, followed by a gradient of 10% solvent B to 90% solvent B over 1 min (solvent A: 100% H₂O with 0.1% formic acid; solvent B: 100% acetonitrile with 0.1% formic acid), with 90% solvent B held for 0.5 min, followed by a return to initial conditions that was maintained for 0.75 min.

To determine the levels of test compound within the SCHH cells, cell plates were removed from the freezer and cell layers lysed in 0.1 mL of methanol containing the internal standard (MeOH-IS), carbamazepine, by shaking for 20 min at room temperature. The lysate (90 μL) was then transferred to a new 96-well plate, dried under a stream of nitrogen, and re-suspended in 90 uL of 50/50 MeOH/H₂O. Standard curves were constructed by adding 0.1 mL of MeOH-IS with known concentrations of analyte (0-500 nM) to vehicle-treated cell layers (matrix blanks). All standards were then processed in the same manner as the unknown samples. For LC-MS/MS analysis the multiple reaction monitoring (MRM) acquisition methods were constructed with tuned transitions for each analyte and the optimal declustering potentials, collision energies, and collision cell exit potentials determined for each analyte with a 4.5 kV spray voltage, 10 eV entrance potential, and 550° C. source temperature. The peak areas of the analyte and internal standard were quantified using Analyst 1.5.2 (MDS SCIEX, Ontario, Canada). The resulting drug levels were then normalized to the hepatocyte protein content in a well as determined by the BCA Protein Assay Kit (Pierce Biotechnology). The data are shown in Table 3.

A humanized PCSK9 mouse model was developed to assess compound activity in vivo. This model was established by first generating a transgenic mouse containing the full-length human PCSK9 gene and its promoter through pronuclear injection of the bacterial artificial chromosome (BAC), RP11-627J9, into C57B16J mice. Mice containing the human PCSK9 transgene were then bred with PCSK9 knockout mice on a 129/C57BL6J background (Rashid S, Curtis D E, Garuti R, et al. Decreased plasma cholesterol and hypersensitivity to statins in mice lacking Pcsk9. Proc Natl Acad Sci USA 2005; 102(15):5374-9). Animals expressing the human transgene that were null for the mouse isoform were put on C57BL6J background by speed congenics. Male mice genotype confirmed to contain the human PCSK9 transgene absent mouse PCSK9 were utilized to profile compounds. These animals are herein referred to as hPCSK9 mice. Animals were maintained on a standard chow diet prior to and during the study in an environment with a 12-hour (h) light-dark cycle and free access to food. To evaluate the ability of compounds to lower plasma PCSK9, the parent compounds were formulated as a solution in a vehicle of 0.5% methylcellulose and administered by oral gavage at doses of 100, 300 and 500 mg/kg. Plasma samples were taken at hour zero (baseline), prior to compound administration and then at 0.5, 1, 2, 4, 8 and 24 h following the single dose for determination of circulating plasma PCSK9 levels as well as measurement of the corresponding concentration of the hydrolyzed active metabolite by mass spectroscopy (MS). In addition to the group of animals used to measure plasma compound and PCSK9 concentrations, a satellite cohort of hPCSK9 transgenic mice were dosed orally at 300 mg/kg and liver samples were collected at 0.5, 1, 2, 4 and 8 h post-gavage to assess liver concentration of the corresponding hydrolyzed active metabolite by MS (the 24 h terminal samples from the plasma arm at all 3 doses were used to source the 24 h time point and to assess dose proportionality exposure within the liver). For example, ethyl (S)-1-{5-[4-(4-{(3-chloropyridin-2-yl)[(3R)-piperidin-3-yl]carbamoyl}-2-fluorophenyl)-1-methyl-1H-pyrazol-5-yl]-1H-tetrazol-1-yl}ethyl carbonate (the parent molecule) was dosed orally and plasma and liver concentrations were measured for the metabolite, N-(3-chloropyridin-2-yl)-3-fluoro-4-[1-methyl-5-(2H-tetrazol-5-yl)-1H-pyrazol-4-yl]-N-[(3R)-piperidin-3-yl]benzamide. Quantitation of human plasma PCSK9 was performed using a commercially available sandwich ELISA kit (R&D Systems, DPC900) incorporating a horse radish peroxidase (HRP) conjugated secondary antibody (R&D Systems, DPC900) to generate a colorimetric signal proportional to PCSK9 concentration per the manufacturer's protocol. Plasma samples taken from the humanized mice were diluted 1:60 placing all samples within the assay's linear range of detection (0.312 to 20 pg/mL). Samples were measured as at least duplicate technical replicates at an absorbance of 450 nm with a reference wavelength of 540 nm on a Spectramax M5e (Molecular Devices). Reduction in plasma PCSK9, attributed to concentrations of the liberated active metabolite, was dose proportional and maximum lowering was observed 4 hours following dosing of the parent compound. Data for the 500 mg/kg treatment groups are summarized in Table 4.

TABLE 1
Human Enterocyte and Hepatocyte Stability Data

| | Hᵢₙₜ CLᵢₙₜ | H_{Hep} CLᵢₙₜ |
|---|---|---|
| Example | (μL/min/mg) | (μL/min/mil) |
| 5a | <57.8 | 57.2 |
| 5b | 86.9 | 85.0 |
| 6 | <57.8 | 71.2 |
| 7 | <82.9 | 97.6 |
| 8 | 116 | 51.3 |
| 9 | <57.8 | 7.0 |
| 10 | <57.8 | 11.8 |
| 11 | 620 | >170 |

TABLE 2
Biological Data

| | Cell Based | Cell Free |
|---|---|---|
| | PCSK9 IC₅₀ | PCSK9 IC₅₀ |
| Example | (μM) | (μM) |
| 1 | >20 | 5.8 |
| 2 | >20 | 10.5 |
| 3 | >20 | 2.8 |
| 4 | >20 | 15.3 |
| 5a | >20 | 8.2 |
| 5b | >20 | 6.4 |
| 6 | >20 | 10.3 |
| 7 | >20 | 13.4 |
| 8 | >20 | 11.0 |
| 9 | >20 | 58.9 |
| 10 | >20 | >74 |
| 11 | 16.1 | |
| 12 | 17.3 | 15.4 |

TABLE 3
Sandwich Culture Human Hepatocyte Biological Data

| | SCHH PCSK9 |
|---|---|
| Example | IC₅₀ (μM) |
| 3 | 63.4 |

TABLE 4
In Vivo PCSK9 Lowering in Humanized PCSK9 Mice

| | Oral Dose | Percent Plasma PCSK9 |
|---|---|---|
| Example | (mg/kg) | Lowering at 4 hours* |
| 6 | 500 | 71 |
| 7 | 500 | 72 |

*Relative to hour zero (baseline) levels

### Global Proteomic Assay-Stable Isotope Labeling of Amino Acids in Cell Culture (SILAC) Assay:

Compound selectivity for the inhibition of translation of PCSK9 mRNA to PCSK9 protein is determined by a global proteomics assay (e.g. SILAC). Human hepatocarcinoma Huh7 cells for stable isotope labeling by amino acids (SILAC) are grown in RPMI media (minus lysine and arginine) in 10% dialyzed fetal bovine serum supplemented with either unlabeled lysine and arginine (light label), L-arginine:HCl U-13C6 99% and L-lysine:2HCl 4,4,5,5-D4, 96-98% (medium label) or L-arginine:HCl U13C6, 99%; U-15N4, 99% and L-lysine:2HCl U13C6, 99%; U-15N2, 99% (heavy label). Cells are passaged for 5-6 doublings with an incorporation efficiency for labeling of >95% achieved. Prior to the start of the experiment, cells are cultured to full confluence to facilitate a synchronized cell population in G0/G1 phase (cell cycle analysis with propidium iodide showed that 75% of cells were in G0/G1 phase). Cells are then re-plated in fresh media supplemented with 0.5% dialyzed fetal bovine serum containing either light, medium or heavy lysine (Lys) and arginine (Arg) and vehicle (light) or test PCSK9 compound 0.25 uM (medium) or 1.30 μM (heavy) for either 1, 4 or 16 hours. At the end of the indicated time points, media is removed and protease/phosphatase inhibitors added prior to freezing at −80° C. Cell layers are rinsed with PBS before adding cell dissociation buffer to detach the cells, cells are collected by rinsing with PBS and spun at 1000 rpm for 5 minutes. The cell pellet is resuspended in PBS for washing, spun at 1000 rpm for 5 minutes and the supernatant aspirated. The cell layer is then frozen at −80° C. and both the media and cell pellet are then subjected to proteomic analysis.

For proteomic analysis of secreted proteins, equal volume of the conditioned media from light, medium, and heavy cells is mixed, followed by depletion of bovine serum albumin by anti-BSA agarose beads. The resulting proteins are then concentrated using 3KDa MWCO spin columns, reduced with dithiothreitol and alkylated with iodoacetamide.

For the analysis of cellular proteins, cell pellets are lysed in SDS-PAGE loading buffer in the presence of protease/phosphatase inhibitor cocktails. Cell lysates are centrifuged at 12 000×g at 4° C. for 10 min. The resulting supernatants are then collected, and protein concentrations measured by BCA assay. Equal amount proteins in the light, medium, and heavy cell lysates are combined, reduced with dithiothreitol and alkylated with iodoacetamide.

The proteins derived from conditioned media and cell pellets are subsequently fractionated by SDS-PAGE. The gels are stained with Coomassie blue and following destaining the gels are cut into 12-15 bands. Proteins are in-gel digested by trypsin overnight, after which peptides are extracted with CH₃CN:1% formic acid (1:1, v/v). The resulting peptide mixtures are then desalted with C₁₈ Stage-Tips, dried in speedvac and stored at −20° C. until further analysis.

The peptide mixtures are reconstituted in 0.1% formic acid. An aliquot of each sample is loaded onto a C₁₈ PicoFrit column (75 μm×10 cm) coupled to an LTQ Orbitrap Velos mass spectrometer. Peptides are separated using a 2-hour linear gradient. The instrumental method consists of a full MS scan followed by data-dependent CID scans of the 20 most intense precursor ions, and dynamic exclusion is activated to maximize the number of ions subjected to fragmentation. Peptide identification and relative protein quantification are carried out by searching the mass spectra against the human IPI database using Mascot search engine on Proteome Discoverer 1.3. The mass spectra for peptides derived from the conditioned media are also searched against bovine IPI database to discern proteins carried over from fetal bovine serum. The search parameters take into account static modification of S-carboxamidomethylation at Cys, and variable modifications of oxidation on Met and stable isotopic labeling on Lys and Arg. Peptide spectrum matches (PSMs) at 1% false discovery rate are used for protein identifications. Changes in protein expression upon compound treatment are calculated from the relative intensity of isotope-labeled and unlabeled peptides derived from that protein. The protein candidates thus identified by the software with altered expression (<=2-fold or 50% decrease) are further validated for accuracy by manual inspection of the MS and MS/MS spectra of the respective peptides and those meeting this criteria are determined to be significantly decreased upon compound treatment.

For administration to human patients, an oral daily dose of the compounds herein may be in the range 1 mg to 5000 mg depending, of course, on the mode of and frequency of administration, the disease state, and the age and condition of the patient, etc. By patient is meant a human, either male or female. The patient may be of any age group including infants (under the age of 2), children (under the age of 12), teenagers (between the ages of 13-19), adults (between the ages of 20-65), pre-menopausal females, post menopausal females and the elderly (over the age of 65). A therapeutically effective amount is about 1 mg to about 4000 mg per day. Preferably the therapeutically effective amount is about 1 mg to about 2000 mg per day. It is especially preferred that a therapeutically effective amount is about 50 mg to about 500 mg per day. An oral daily dose is in the range of 3 mg to 2000 mg may be used. A further oral daily dose is in the range of 5 mg to 1000 mg. For convenience, the compounds of the present invention can be administered in a unit dosage form. If desired, multiple doses per day of the unit dosage form can be used to increase the total daily dose. The unit dosage form, for example, may be a tablet or capsule containing about 0.1, 0.5, 1, 5, 10, 15, 20, 25, 30, 35, 40, 45, 50, 55, 60, 65, 70, 75, 80, 85, 90, 95, 100, 125, 150, 175, 200, 250, 500, 1000 or 2000 mg of the compound of the present invention. The total daily dose may be administered in single or divided doses and may, at the physician's discretion, fall outside of the typical ranges given herein.

For administration to human patients, an infusion daily dose of the compounds herein may be in the range 1 mg to 2000 mg depending, of course, on the mode of and frequency of administration, the disease state, and the age and condition of the patient, etc. A further infusion daily dose is in the range of 5 mg to 1000 mg. The total daily dose may be administered in single or divided doses and may, at the physician's discretion, fall outside of the typical ranges given herein.

These compounds may also be administered to animals other than humans, for example, for the indications detailed above. The precise dosage administered of each active ingredient will vary depending upon any number of factors, including but not limited to, the type of animal and type of disease state being treated, the age of the animal, and the route(s) of administration.

A dosage of the combination pharmaceutical agents to be used in conjunction with the Formula I compounds is used that is effective for the indication being treated. Such dosages can be determined by standard assays such as those referenced above and provided herein. The combination agents may be administered simultaneously or sequentially in any order.

These dosages are based on an average human subject having a weight of about 60 kg to 70 kg. The physician will readily be able to determine doses for subjects whose weight falls outside this range, such as infants and the elderly.

Dosage regimens may be adjusted to provide the optimum desired response. For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form, as used herein, refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the chemotherapeutic agent and the particular therapeutic or prophylactic effect to be achieved, and (b) the limitations inherent in the art of compounding such an active compound for the treatment of sensitivity in individuals.

Thus, one of skill in the art would appreciate, based upon the disclosure provided herein, that the dose and dosing regimen is adjusted in accordance with methods well-known in the therapeutic arts. That is, the maximum tolerable dose can be readily established, and the effective amount providing a detectable therapeutic benefit to a patient may also be determined, as can the temporal requirements for administering each agent to provide a detectable therapeutic benefit to the patient. Accordingly, while certain dose and administration regimens are exemplified herein, these examples in no way limit the dose and administration regimen that may be provided to a patient in practicing the present invention.

It is to be noted that dosage values may vary with the type and severity of the condition to be alleviated, and may include single or multiple doses. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition. For example, doses may be adjusted based on pharmacokinetic or pharmacodynamic parameters, which may include clinical effects such as toxic effects and/or laboratory values. Thus, the present invention encompasses intra-patient dose-escalation as determined by the skilled artisan. Determining appropriate dosages and regiments for administration of the chemotherapeutic agent are well-known in the relevant art and would be understood to be encompassed by the skilled artisan once provided the teachings disclosed herein.

The present invention further comprises use of a compound of Formula I for use as a medicament (such as a unit dosage tablet or unit dosage capsule). In another embodiment, the present invention comprises the use of a compound of Formula I for the manufacture of a medicament (such as a unit dosage tablet or unit dosage capsule) to treat one or more of the conditions previously identified in the above sections discussing methods of treatment.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The compounds described herein may be administered as a formulation comprising a pharmaceutically effective amount of a compound of Formula I, in association with one or more pharmaceutically acceptable excipients including carriers, vehicles and diluents. The term “excipient” herein means any substance, not itself a therapeutic agent, used as a diluent, adjuvant, or vehicle for delivery of a therapeutic agent to a subject or added to a pharmaceutical composition to improve its handling or storage properties or to permit or facilitate formation of a solid dosage form such as a tablet, capsule, or a solution or suspension suitable for oral, parenteral, intradermal, subcutaneous, or topical application. Excipients can include, by way of illustration and not limitation, diluents, disintegrants, binding agents, adhesives, wetting agents, polymers, lubricants, glidants, stabilizers, and substances added to mask or counteract a disagreeable taste or odor, flavors, dyes, fragrances, and substances added to improve appearance of the composition. Acceptable excipients include (but are not limited to) stearic acid, magnesium stearate, magnesium oxide, sodium and calcium salts of phosphoric and sulfuric acids, magnesium carbonate, talc, gelatin, acacia gum, sodium alginate, pectin, dextrin, mannitol, sorbitol, lactose, sucrose, starches, gelatin, cellulosic materials, such as cellulose esters of alkanoic acids and cellulose alkyl esters, low melting wax, cocoa butter or powder, polymers such as polyvinyl-pyrrolidone, polyvinyl alcohol, and polyethylene glycols, and other pharmaceutically acceptable materials. Examples of excipients and their use may be found in Remington's Pharmaceutical Sciences, 20th Edition (Lippincott Williams & Wilkins, 2000). The choice of excipient will to a large extent depend on factors such as the particular mode of administration, the effect of the excipient on solubility and stability, and the nature of the dosage form.

The compounds herein may be formulated for oral, buccal, intranasal, parenteral (e.g., intravenous, intramuscular or subcutaneous) or rectal administration or in a form suitable for administration by inhalation. The compounds of the invention may also be formulated for sustained delivery.

Methods of preparing various pharmaceutical compositions with a certain amount of active ingredient are known, or will be apparent in light of this disclosure, to those skilled in this art. For examples of methods of preparing pharmaceutical compositions see Remington's Pharmaceutical Sciences, 20th Edition (Lippincott Williams & Wilkins, 2000).

Pharmaceutical compositions according to the invention may contain 0.1%-95% of the compound(s) of this invention, preferably 1%-70%. In any event, the composition to be administered will contain a quantity of a compound(s) according to the invention in an amount effective to treat the disease/condition of the subject being treated.

Since the present invention has an aspect that relates to the treatment of the disease/conditions described herein with a combination of active ingredients which may be administered separately, the invention also relates to combining separate pharmaceutical compositions in kit form. The kit comprises two separate pharmaceutical compositions: a compound of Formula I a prodrug thereof or a salt of such compound or prodrug and a second compound as described above. The kit comprises a means for containing the separate compositions such as a container, a divided bottle or a divided foil packet. Typically the kit comprises directions for the administration of the separate components. The kit form is particularly advantageous when the separate components are preferably administered in different dosage forms (e.g., oral and parenteral), are administered at different dosage intervals, or when titration of the individual components of the combination is desired by the prescribing physician.

An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, etc. . . . Second Week, Monday, Tuesday, . . . ” etc. Other variations of memory aids will be readily apparent. A “daily dose” can be a single tablet or capsule or several pills or capsules to be taken on a given day. Also, a daily dose of Formula I compound can consist of one tablet or capsule while a daily dose of the second compound can consist of several tablets or capsules and vice versa. The memory aid should reflect this.

In another specific embodiment of the invention, a dispenser designed to dispense the daily doses one at a time in the order of their intended use is provided. Preferably, the dispenser is equipped with a memory-aid, so as to further facilitate compliance with the regimen. An example of such a memory-aid is a mechanical counter which indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

Also, as the present invention has an aspect that relates to the treatment of the disease/conditions described herein with a combination of active ingredients which may be administered jointly, the invention also relates to combining separate pharmaceutical compositions in a single dosage form, such as (but not limited to) a single tablet or capsule, a bilayer or multilayer tablet or capsule, or through the use of segregated components or compartments within a tablet or capsule.

The active ingredient may be delivered as a solution in an aqueous or non-aqueous vehicle, with or without additional solvents, co-solvents, excipients, or complexation agents selected from pharmaceutically acceptable diluents, excipients, vehicles, or carriers.

The active ingredient may be formulated as an immediate release or modified release tablet or capsule. Alternatively, the active ingredient may be delivered as the active ingredient alone within a capsule shell, without additional excipients.

### General Experimental Procedures

The following examples are put forth so as to provide those of ordinary skill in the art with a disclosure and description of how the compounds, compositions, and methods claimed herein are made and evaluated, and are intended to be purely exemplary of the invention and are not intended to limit the scope of what the inventors regard as their invention. Unless indicated otherwise, percent is percent by weight given the component and the total weight of the composition, temperature is in ° C. or is at ambient temperature, and pressure is at or near atmospheric. Commercial reagents were utilized without further purification. Room or ambient temperature refers to 18-25° C. All non-aqueous reactions were run under a nitrogen atmosphere for convenience and to maximize yields. Concentration in vacuo means that a rotary evaporator was used. The names for the compounds of the invention were created by the Autonom 2.0 PC-batch version from Beilstein Informationssysteme GmbH (ISBN 3-89536-976-4). “DMSO” means dimethyl sulfoxide.

Proton nuclear magnetic spectroscopy (¹H NMR) was recorded with 400 and 500 MHz spectrometers. Chemical shifts are expressed in parts per million downfield from tetramethylsilane. The peak shapes are denoted as follows: s, singlet; d, doublet; t, triplet; q, quartet; m, multiplet; br s, broad singlet; br m, broad multiplet. Mass spectrometry (MS) was performed via atmospheric pressure chemical ionization (APCI) or electron scatter (ES) ionization sources. Silica gel chromatography was performed primarily using a medium pressure system using columns pre-packaged by various commercial vendors. Microanalyses were performed by Quantitative Technologies Inc. and were within 0.4% of the calculated values. The terms “concentrated” and “evaporated” refer to the removal of solvent at reduced pressure on a rotary evaporator with a bath temperature less than 60° C. The abbreviation “min” and “h” stand for “minutes” and “hours” respectively. The abbreviation “g” stands for grams. The abbreviation “μl” or “μL” or “uL” stand for microliters.

The powder X-ray diffraction was carried out on a Bruker AXS-D4 diffractometer using copper radiation (wavelength: 1.54056 Å). The tube voltage and amperage were set to 40 kV and 40 mA, respectively. The divergence and scattering slits were set at 1 mm, and the receiving slit was set at 0.6 mm. Diffracted radiation was detected by a PSD-Lynx Eye detector. A step size of 0.02° and a step time of 0.3 sec from 3.0 to 40° 2θ were used. Data were collected and analyzed using Bruker Diffrac Plus software (Version 2.6). Samples were prepared by placing them in a customized holder and rotated during collection.

To perform an X-ray diffraction measurement on a Bragg-Brentano instrument like the Bruker system used for measurements reported herein, the sample is typically placed into a holder which has a cavity. The sample powder is pressed by a glass slide or equivalent to ensure a random surface and proper sample height. The sample holder is then placed into the instrument. The incident X-ray beam is directed at the sample, initially at a small angle relative to the plane of the holder, and then moved through an arc that continuously increases the angle between the incident beam and the plane of the holder. Measurement differences associated with such X-ray powder analyses result from a variety of factors including: (a) errors in sample preparation (e.g., sample height), (b) instrument errors (e.g. flat sample errors), (c) calibration errors, (d) operator errors (including those errors present when determining the peak locations), and (e) the nature of the material (e.g. preferred orientation and transparency errors). Calibration errors and sample height errors often result in a shift of all the peaks in the same direction. Small differences in sample height when using a flat holder will lead to large displacements in XRPD peak positions. A systematic study showed that, using a Shimadzu XRD-6000 in the typical Bragg-Brentano configuration, sample height difference of 1 mm lead to peak shifts as high as 1°2θ (Chen et al.; J Pharmaceutical and Biomedical Analysis, 2001; 26, 63). These shifts can be identified from the X-ray Diffractogram and can be eliminated by compensating for the shift (applying a systematic correction factor to all peak position values) or recalibrating the instrument. As mentioned above, it is possible to rectify measurements from the various machines by applying a systematic correction factor to bring the peak positions into agreement. In general, this correction factor will bring the measured peak positions from the Bruker into agreement with the expected peak positions and may be in the range of 0 to 0.2° 2θ.

### Analytical UPLC-MS Method 1:

### Column: Waters Acquity HSS T3, C₁₈ 2.1×5 0 mm, 1.7 μm; Column T=60° C.

Gradient: Initial conditions: A-95%:B-5%; hold at initial from 0.0-0.1 min; Linear Ramp to A-5%:B-95% over 0.1-1.0 min; hold at A-5%:B-95% from 1.0-1.1 min; return to initial conditions 1.1-1.5 min
Mobile Phase A: 0.1% formic acid in water (v/v)
Mobile Phase B: 0.1% formic acid in acetonitrile (v/v)
Flow rate: 1.25 mL/min

### Analytical UPLC-MS Method 2:

### Column: Waters Acquity HSS T3, C₁₈ 2.1×5 0 mm, 1.7 μm; Column T=60° C.

Gradient: Initial conditions: A-95%:B-5%; hold at initial from 0.0-0.1 min; Linear Ramp to A-5%:B-95% over 0.1-2.6 min; hold at A-5%:B-95% from 2.6-2.95 min; return to initial conditions 2.95-3.0 min
Mobile Phase A: 0.1% formic acid in water (v/v)
Mobile Phase B: 0.1% formic acid in acetonitrile (v/v)
Flow rate: 1.25 mL/min

### Analytical LC-MS Method 3:

Column: Welch Materials Xtimate 2.1 mm×30 mm, 3 μm
Gradient: 0-60% (solvent B) over 2.0 min
Mobile Phase A: 0.0375% TFA in water
Mobile Phase B: 0.01875% TFA in acetonitrile
Flow rate: 1.2 mL/min

### Chiral Preparative Chromatography Method 1:

Column: Chiralpak IC 2.1 cm×25 cm, 5 μm

### Mobile Phase: 85/15 CO₂/methanol

Flow Rate: 65 mL/min

### Column Temp: Ambient

### Wavelength: 280 nm

### Injection Volume: 2.0 mL

### Feed Concentration: 125 g/L

### Chiral Preparative Chromatography Method 2:

Column: Chiral Tech AD-H 250 mm×21.2 mm, 5 μm; Column T=ambient
Mobile Phase: 80% CO₂/20% methanol; isocratic conditions
Flow Rate: 80.0 mL/min

### Chiral Preparative Chromatography Method 3:

Column: ChiralPak AD 5 cm×25 cm, 5 μm

### Mobile Phase: 90/10 CO₂/methanol

Flow Rate: 250 mL/min

### Column Temp: 35° C.

### Wavelength: 254 nm

### Injection Volume: 4.5 mL

### Feed Concentration: 100 g/L

### Chiral Analytical Chromatography Method 1

Column: Chiral Tech AD-H 250 mm×4.6 mm, 5 μm
Gradient: Initial conditions: A-95%:B-5%; linear ramp to A-40%:B-60% over 1.0-9.0 min; hold at A-40%:B-60% from 9.0-9.5 min; linear ramp to A-95%:B-5% over 9.5-10.0 min.

### Mobile Phase A: CO₂

Mobile Phase B: methanol
Flow rate: 3.0 mL/min

### Detection: UV-210 nm

### PREPARATIONS

### Preparation 1: tert-butyl (3R)-3-[(3-chloropyridin-2-yl)amino]piperidine-1-carboxylate

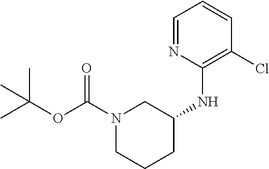 [SMILES: CC(C)(C)OC(=O)N1CCC[C@@H](Nc2ncccc2Cl)C1]

A mixture of 2-bromo-3-chloropyridine (203.8 g, 1.06 moles), sodium tert-amylate (147 g, 1.27 moles), tert-butyl (3R)-3-aminopiperidine-1-carboxylate (249.5 g, 1.25 moles) in toluene (2 L) was heated to 80° C. To this solution was added chloro(di-2-norbornylphosphino)(2-dimethylaminoferrocen-1-yl)palladium (II) (6.1 g, 10.06 mmol) followed by heating to 105° C. and holding for 3 h. The reaction mixture was cooled to room temperature, 1 L of water was added, then the biphasic mixture was filtered through Celite®. After layer separation, the organic phase was washed with 1 L of water followed by treatment with 60 g of Darco® G-60 at 50° C. The mixture was filtered through Celite®, and concentrated to a final total volume 450 mL, resulting in the precipitation of solids. To the slurry of solids was added 1 L of heptane. The solids were collected via filtration and then dried to afford the title compound as a dull orange solid (240.9 g, 73% yield).

¹H NMR (CDCl₃) δ: 8.03 (m, 1H), 7.45 (m, 1H), 6.54 (m, 1H), 5.08 (br s, 1H), 4.14 (br s, 1H), 3.85-3.30 (m, 4H), 2.00-1.90 (m, 1H), 1.80-1.55 (m, 4H), 1.43 (br s, 9H).

UPLC (UPLC-MS Method 1): t_{R}=0.72 min.

MS (ES+) 312.0 (M+H)⁺

### Preparation 2: tert-butyl (3R)-3-[(3-methylpyridin-2-yl)amino]piperidine-1-carboxylate

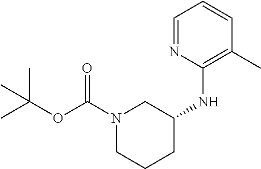 [SMILES: Cc1cccnc1N[C@@H]1CCCN(C(=O)OC(C)(C)C)C1]

To a solution of 2-bromo-3-methylpyridine (75.0 g, 436 mmol) and tert-butyl (3R)-3-aminopiperidine-1-carboxylate (87.3 g, 436 mmol) in toluene (1.2 L) were added Cs₂CO₃ (426 g, 1.31 mol), 2-(dimethylaminomethyl)ferrocen-1-yl-palladium(II) chloride dinorbornylphosphine (MFCD05861622) (1.56 g, 4.36 mmol) and Pd(OAc)₂ (0.490 g, 2.18 mmol) under N₂ atmosphere. The mixture was stirred at 110° C. for 48 h. The mixture was cooled to room temperature then poured into water (500 mL) and extracted with EtOAc (3×300 mL). The organic layers were dried over Na₂SO₄, filtered, and the filtrate was concentrated in vacuo. The residue was purified by silica gel column chromatography to give the title compound as a yellow solid (65 g, 60%).

¹H NMR (CDCl₃) δ: 8.00 (d, 1H), 7.20 (d, 1H), 6.51 (dd, 1H), 4.36 (br s, 1H), 4.16 (br s, 1H), 3.63 (d, 1H), 3.52 (br s, 2H), 3.36-3.30 (m, 1H), 2.06 (s, 3H), 1.90 (br s, 1H), 1.73 (br s 2H), 1.59 (br s, 1H), 1.38 (br s, 9H).

### Preparation 3: tert-butyl (3R)-3-[(4-bromobenzoyl)(3-chloropyridin-2-yl)amino]piperidine-1-carboxylate

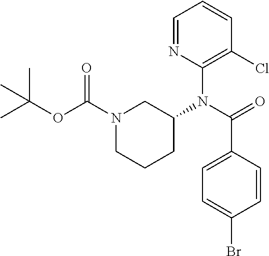 [SMILES: CC(C)(C)OC(=O)N1CCC[C@@H](N(C(=O)c2ccc(Br)cc2)c2ncccc2Cl)C1]

Preparation 1 tert-Butyl (3R)-3-[(3-chloropyridin-2-yl)amino]piperidine-1-carboxylate (214.4 g, 687.7 mmol) was dissolved in 260 mL of THF and the resulting suspension was cooled to −10° C. Lithium bis(trimethylsilyl)amide (1 mol/L in THF, 687.7 mL, 687.1 mmol) was added over 25 min followed by warming to 20° C. and stirring for 1 h before cooling back to −10° C. 4-Bromobenzoyl chloride (140.0 g, 625.2 mmol) was added as a solution in 230 mL of THF over 1.5 h, maintaining the internal temperature at less than −7° C. After complete addition, the reaction mixture was warmed to 0° C. at which point HPLC indicated the reaction was complete. MeOH was added (101 mL), then the reaction was warmed to room temperature and concentrated in vacuo to a low volume. The solvent was then exchanged to 2-MeTHF. The crude product solution (700 mL in 2-MeTHF) was washed with 700 mL of half-saturated aqueous NaHCO₃, followed by 200 mL of half-saturated brine. The 2-MeTHF solution was concentrated to a low volume followed by addition of 400 mL of heptane resulting in precipitation of solids which were collected via filtration. The collected solids were dried to afford the title compound as a tan powder (244 g, 79% yield).

¹H NMR (acetonitrile-d₃) δ: 8.57-8.41 (m, 1H), 7.85-7.62 (m, 1H), 7.37 (d, 2H), 7.31 (dd, 1H), 7.23 (d, 2H), 4.63-4.17 (m, 2H), 4.06-3.89 (m, 1H), 3.35-3.08 (br s, 0.5H), 2.67-2.46 (m, 1H), 2.26-2.10 (br s, 0.5H), 1.92-1.51 (m, 3H), 1.46 (s, 9H), 1.37-1.21 (m, 1H).

UPLC (UPLC Method 3): t_{R}=7.03 min.

### Alternative Method for Preparation 3:

To a solution of Preparation 1 (R)-tert-butyl 3-((3-chloropyridin-2-yl)amino)piperidine-1-carboxylate (100 g, 321 mmol) and 4-bromobenzoyl chloride (73.7 g, 336 mmol) in dry THF (500 mL) was added 1 M lithium bis(trimethylsilyl)amide (362 mL, 362 mmol) dropwise at 0° C. The reaction mixture was warmed and stirred at room temperature overnight. The reaction was quenched with water and extracted with EtOAc (3×1000 mL). The combined organic layers were washed with brine, dried over Na₂SO₄, filtered and the filtrate was concentrated in vacuo. The residue was purified by chromatography on silica gel to give afford the title compound as a yellow solid (100 g, 63%).

¹H NMR (CDCl₃) δ: 8.43 (br s, 1H), 7.56 (br s, 1H), 7.28-7.14 (m, 5H), 4.48 (br s, 2H), 4.24 (br s, 1H), 4.09 (br s, 1H), 3.28 (br s, 1H), 2.54 (br s, 1H), 2.27 (br s, 1H), 1.63-1.54 (br m, 1H), 1.46 (br s, 10H).

### Preparation 4: tert-butyl (3R)-3-[(4-bromobenzoyl)(3-methylpyridin-2-yl)amino]piperidine-1-carboxylate

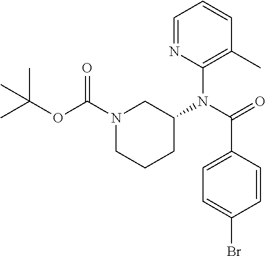 [SMILES: Cc1cccnc1N(C(=O)c1ccc(Br)cc1)[C@@H]1CCCN(C(=O)OC(C)(C)C)C1]

To a solution of Preparation 2 (R)-tert-butyl 3-((3-methylpyridin-2-yl)amino)piperidine-1-carboxylate (33.3 g, 114 mmol) and 4-bromobenzoyl chloride (26.3 g, 120 mmol) in dry THF (300 mL) was added 1 M lithium bis(trimethylsilyl)amide (137 mL, 137 mmol) dropwise at 0° C. The reaction mixture was warmed and stirred at room temperature for 16 h. The reaction was quenched with water and extracted with EtOAc (3×1000 mL). The combined organic layers were washed with brine, dried over Na₂SO₄, filtered, and the filtrate was concentrated in vacuo. The residue was purified by silica gel chromatography to afford the title compound as a yellow solid (27 g, 50%).

¹H NMR (CDCl₃) δ: 8.41 (br s, 1H), 7.34 (br s, 1H), 7.25 (d, 2H), 7.16-7.14 (m, 3H), 4.65 (br s, 1H), 4.48 (br d, 1H), 4.15-4.04 (br m, 2H), 3.39 (br s, 1H), 2.55 (br s, 1H), 2.37 (br s, 1H), 2.01-1.98 (br d, 3H), 1.74 (br s, 1H), 1.47-1.43 (br d, 10H).

### Preparation 5: tert-butyl (3R)-3-[(4-bromo-3-fluorobenzoyl)(3-methylpyridin-2-yl)amino]piperidine-1-carboxylate

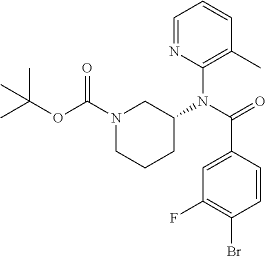 [SMILES: Cc1cccnc1N(C(=O)c1ccc(Br)c(F)c1)[C@@H]1CCCN(C(=O)OC(C)(C)C)C1]

To a solution of Preparation 2 (R)-tert-butyl 3-((3-methylpyridin-2-yl)amino)piperidine-1-carboxylate (30 g, 100 mmol) in dry THF (150 mL) was added 1 M lithium bis(trimethylsilyl)amide (129 mL, 129 mmol) dropwise at 0° C. A solution of and 4-bromo-3-fluorobenzoyl chloride (31.8 g, 134 mmol) in dry THF (100 mL) was added dropwise at 0° C. After 2 h, the reaction mixture was warmed and stirred at room temperature for 1 h. The reaction was cooled to 0° C., quenched with water and extracted with EtOAc (3×500 mL). The combined organic layers were washed with brine, dried over Na₂SO₄, filtered, and the filtrate was concentrated in vacuo. The residue was purified by silica gel chromatography to afford the title compound as a white solid (40 g, 79%).

¹H NMR (MeOH-d4, mixture of rotomers) δ: 8.5-8.4 (br s, 1H), 7.68-7.53 (br s, 1H), 7.45 (dd, 1H), 7.29 (dd, 1H), 7.12 (d, 1H), 7.00 (d, 1H), 4.60-4.45 (br s, 2H), 4.25-3.95 (br m, 2H), 3.44-3.34 (br m, 1H), 2.75-2.55 (br m, 1H), 2.35-2.05 (br m, 1H), 2.16 and 2.07 (s, 3H), 1.85-1.65 (br m, 1H), 1.65-1.35 (br m, 1H), 1.50 and 1.42 (br s, 9H).

### Preparation 6: tert-butyl (3R)-3-[(4-bromo-3-fluorobenzoyl)(3-chloropyridin-2-yl)amino]piperidine-1-carboxylate

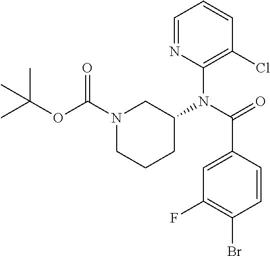 [SMILES: CC(C)(C)OC(=O)N1CCC[C@@H](N(C(=O)c2ccc(Br)c(F)c2)c2ncccc2Cl)C1]

To a solution of Preparation 1 (R)-tert-butyl 3-((3-chloropyridin-2-yl)amino)piperidine-1-carboxylate (35 g, 112 mmol) in dry THF (500 mL) was added 1 M lithium bis(trimethylsilyl)amide (140 mL, 140 mmol) dropwise at 0° C. A solution of and 4-bromo-3-fluorobenzoyl chloride (35 g, 147 mmol) in dichloromethane (100 mL) was added dropwise at 0° C. After 20 min, the reaction mixture was warmed and stirred at room temperature for 18 h. The reaction was quenched with saturated NH₄Cl, poured into water (300 mL) and extracted with EtOAc (2×200 mL). The combined organic layers were washed with brine, dried over Na₂SO₄, filtered, and the filtrate was concentrated in vacuo. The residue was purified by silica gel chromatography to afford the title compound as a yellow solid (44 g, 76%).

¹H NMR (CDCl₃) δ: 8.46 (br s, 1H), 7.61 (br s, 1H), 7.37-7.30 (m, 1H), 7.24-7.18 (m, 1H), 7.12 (d, 1H), 6.97 (d, 1H), 4.65-4.39 (br m, 5H), 3.35-3.22 (br m, 1H), 2.70-1.90 (br m, 3H), 1.47 (br s, 9H).

### Preparation 7

### tert-butyl (3R)-3-{[(5-bromopyridin-2-yl)carbonyl](3-chloropyridin-2-yl)amino}piperidine-1-carboxylate

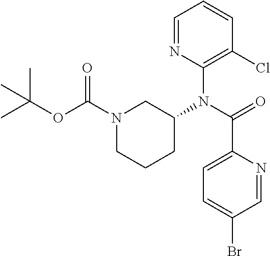 [SMILES: CC(C)(C)OC(=O)N1CCC[C@@H](N(C(=O)c2ccc(Br)cn2)c2ncccc2Cl)C1]

Two equivalent batches were run in parallel and combined for work-up and purification. To a solution of Preparation 1 (R)-tert-butyl 3-((3-chloropyridin-2-yl)amino)piperidine-1-carboxylate (70 g, 224.5 mmol) in dry toluene (1300 mL) was added MeMgCl in THF (3M, 89.8 mL, 269 mmol). After 1 h, methyl 5-bromopicolinate (48.5 g, 224 mmol, MFCD04112493) was added in portions. The reaction mixture was warmed and stirred at room temperature for 64 h. The reaction was quenched with water and combined with the second batch. The mixture of combined batches was extracted with EtOAc (3×300 mL). The combined organic layers were washed with brine, dried over Na₂SO₄, filtered, and the filtrate was concentrated in vacuo. The residue was purified by silica gel chromatography to afford the title compound as a yellow solid (126 g, 57%).

¹H NMR (MeOH-d4, mixture of rotomers) δ: 8.40-8.30 (br m, 1H), 8.25-8.20 (br s, 1H), 8.05-7.95 (m, 2H), 7.90-7.65 (m, 1H), 7.35 (dd, 1H), 4.55-4.45 (br m, 2H), 4.40-4.20 (br m, 1H), 4.10-3.95 (br m, 2H), 3.00-2.50 (br m, 1H), 2.30-1.50 (br m, 3H), 1.50 and 1.45 (br s, 9H).

### Preparation 8

### tert-butyl (3R)-3-{[(5-bromopyridin-2-yl)carbonyl](3-methylpyridin-2-yl)amino}piperidine-1-carboxylate

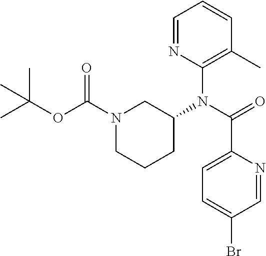 [SMILES: Cc1cccnc1N(C(=O)c1ccc(Br)cn1)[C@@H]1CCCN(C(=O)OC(C)(C)C)C1]

Two equivalent batches were run in parallel and combined for work-up and purification. To a solution of Preparation 2 (R)-tert-butyl 3-((3-methylpyridin-2-yl)amino)piperidine-1-carboxylate (68 g, 233.4 mmol) in dry toluene (750 mL) was added MeMgCl in THF (3M, 93.3 mL, 280 mmol). After 30 min, methyl 5-bromopicolinate (50.4 g, 233 mmol, MFCD04112493) was added in portions. The reaction mixture was stirred at 30-40° C. for 4 h then room temperature for 15 h. The reaction was quenched at 0° C. with water and extracted with EtOAc (2×300 mL). The combined organic layers were washed with brine, dried over Na₂SO₄, filtered, and the filtrate was concentrated in vacuo. The residue was purified by silica gel chromatography to afford the title compound as a yellow solid (130.5 g, 58.5%).

¹H NMR (MeOH-d4, mixtures of rotomers) δ: 8.3-8.20 (br m, 2H), 8.00-7.90 (br s, 1H), 7.65-7.45 (m, 2H), 7.35-7.25 (m, 1H), 4.50 (br d, 1H), 4.45-4.25 (br m, 2H), 4.15-3.95 (br m, 2H), 3.45-3.40 (m, 0.5H), 2.75-2.50 (m, 0.5H), 2.35 and 2.20 (br s, 3H), 2.00-1.40 (br m, 3H), 1.50 and 1.45 (br s, 9H).

### Preparation 9: tert-butyl (3R)-3-{(3-chloropyridin-2-yl)[4-(4,4,5,5-tetramethyl-1,3,2-dioxaborolan-2-yl)benzoyl]amino}piperidine-1-carboxylate

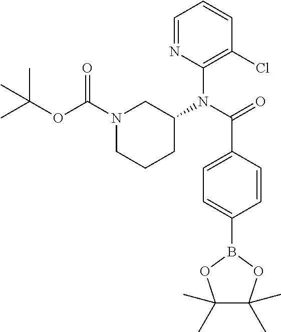 [SMILES: CC(C)(C)OC(=O)N1CCC[C@@H](N(C(=O)c2ccc(B3OC(C)(C)C(C)(C)O3)cc2)c2ncccc2Cl)C1]

To a solution of Preparation 3 (R)-tert-butyl 3-(4-bromo-N-(3-chloropyridin-2-yl)benzamido)piperidine-1-carboxylate (40.0 g, 80.8 mmol) in 1,4-dioxane (250 mL) were added bis(pinacolato)diboron (41.1 g, 162 mmol), KOAc (23.8 g, 244 mmol) and PdCl₂(dppf) (5.9 g, 8.1 mmol). The resulting mixture was purged with N₂ and stirred at 80-90° C. for 10 h. The reaction was cooled and filtered. The organic solution was concentrated in vacuo. The residue was purified by silica gel column chromatography, eluting with a gradient of 2-25% EtOAc/petroleum ether to give the title compound as a yellow gum. The yellow gum was triturated with petroleum ether to afford the title compound as a white solid (30 g, 69%).

¹H NMR (MeOH-d₄) δ: 8.52 (br s, 1H), 7.74 (br s, 1H), 7.55 (br s, 2H), 7.31 (br s, 3H), 4.53 (br s, 1H), 4.30 (br s, 1H), 4.05-4.02 (br m, 1H), 2.80-2.29 (br m, 2H), 1.95-1.68 (m, 3H), 1.50 (br s, 10H), 1.32 (br s, 12H).

### Preparation 10: tert-butyl (3R)-3-{(3-methylpyridin-2-yl)[4-(4,4,5,5-tetramethyl-1,3,2-dioxaborolan-2-yl)benzoyl]amino}piperidine-1-carboxylate

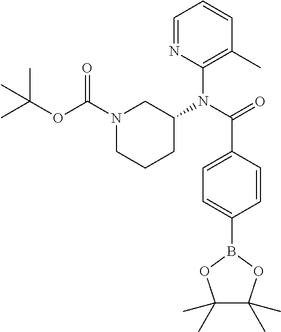 [SMILES: Cc1cccnc1N(C(=O)c1ccc(B2OC(C)(C)C(C)(C)O2)cc1)[C@@H]1CCCN(C(=O)OC(C)(C)C)C1]

A round-bottom flask was charged with Preparation 4, tert-butyl (3R)-3-[(4-bromobenzoyl)(3-methylpyridin-2-yl)amino]piperidine-1-carboxylate (150 g, 317 mmol), bis(pinacolato)diboron (97.8 g, 381 mmol), potassium acetate (100 g, 1.01 mol, and 2-methyltetrahydrofuran (750 mL). The reaction mixture was warmed to 75° C. 1,1′-bis(diphenylphosphino)ferrocene-palladium(II)dichloride dichloromethane complex (Pd(dppf)Cl₂CH₂Cl₂) (5.12 g, 6.21 mmol) was added and the reaction mixture was heated under reflux for 19 h. The reaction mixture was cooled to room temperature and H₂O was added. The reaction mixture was passed through a pad of Celite and the layers separated. The organic layer was concentrated in vacuo. The brown residue was purified by column chromatography on silica gel, eluting with a gradient of 30-50% EtOAc in heptane. The product-containing fractions were concentrated in vacuo. The residue was filtered through a pad of Celite using warm heptane and DCM to solubilize product. The reaction mixture was concentrated in vacuo until product started to crystallize. The solids were granulated for 16 h at room temperature, collected via filtration and dried in a vacuum oven to afford tert-butyl (3R)-3-{(3-methylpyridin-2-yl)[4-(4,4,5,5-tetramethyl-1,3,2-dioxaborolan-2-yl)benzoyl]amino}piperidine-1-carboxylate as a light pink solid (142 g, 86%).

¹H NMR (CDCl₃) δ: 8.40 (m, 1H), 7.53-7.27 (m, 5H), 7.14-6.92 (m, 1H), 4.75-4.45 (m, 2H), 4.20-3.90 (m, 1H), 3.63-3.21 (m, 1H), 2.84-2.10 (m, 3H), 2.06-1.88 (m, 3H), 1.81-1.56 (m, 2H), 1.53-1.37 (m, 9H), 1.31 (s, 12H).

UPLC (UPLC-MS Method 1): t_{R}=1.08 min.

MS (ES+): 522.4 (M+H)⁺.

### Preparation 11: 4-iodo-1-methyl-1H-pyrazole-5-carboxamide

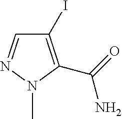

A round-bottom flash was charged with 4-iodo-1-methyl-1H-pyrazole-5-carboxylic acid (297 g, 1.18 mol), DCM (2.97 L), and 1,1′-carbonyldiimidazole (CU) (207 g, 97% by mass, 1.24 mol). The reaction mixture was stirred at room temperature for 45 min. Ammonium chloride (189 g, 3.53 mol) and triethylamine (498 mL, 3.53 mol) were added and the reaction mixture was stirred at room temperature overnight. The reaction mixture was concentrated in vacuo and the residue was suspended in H₂O (˜3 L) and granulated at room temperature for 1 h. The solid was collected via filtration, washed with H₂O, and dried in a vacuum oven to afford 4-iodo-1-methyl-1H-pyrazole-5-carboxamide as a colorless solid (222 g, 75% yield).

¹H NMR (CDCl₃) δ: 7.53 (s, 1H), 6.56 (br s, 1H), 6.01 (br s, 1H), 4.21 (s, 3H).

UPLC (UPLC-MS Method 1): t_{R}=0.15 min.

MS (ES+): 251.1 (M+H)⁺.

### Preparation 12: 4-iodo-1-methyl-1H-pyrazole-5-carbonitrile

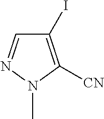

A round-bottom flash was charged with Preparation 11, 4-iodo-1-methyl-1H-pyrazole-5-carboxamide (222 g, 886 mmol) and DCM (2.22 L) and the reaction mixture was cooled to 0° C. 2,6-Lutidine (310 mL, 2.66 mol) and trifluoroacetic anhydride (253 mL, 1.77 mol) were added. After reaction was complete, saturated aqueous sodium bicarbonate (800 mL) was added and the layers separated. The aqueous layer was washed with DCM (800 mL). The organic layers were combined and washed with saturated aqueous ammonium chloride (800 mL), 1N HCl (800 mL), and brine (800 mL). The organic layer was dried over magnesium sulfate, filtered, and concentrated in vacuo. The residue was suspended in heptanes (˜2 L) and granulated at 0-5° C. for 30 min. The solid was collected via filtration and dried in a vacuum oven to afford 4-iodo-1-methyl-1H-pyrazole-5-carbonitrile as a colorless solid (196 g, 95% yield).

¹H NMR (CDCl₃) δ: 7.60 (s, 1H), 4.09 (s, 3H).

UPLC (UPLC-MS Method 1): t_{R}=0.70 min.

MS (ES+): 233.8 (M+H)⁺.

### Preparation 13: 5-(4-iodo-1-methyl-1H-pyrazol-5-yl)-2H-tetrazole

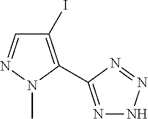

Caution: This reaction generates hydrazoic acid and requires appropriate safety measures.

A reaction vessel was charged with DMF (1.225 L), Preparation 12, 4-iodo-1-methyl-1H-pyrazole-5-carbonitrile (175 g, 751 mmol), sodium azide (147 g, 2.25 mol), and ammonium chloride (121 g, 2.25 mol). H₂O (525 mL) was added slowly to minimize exotherm. The reaction mixture was heated at 100° C. overnight. The reaction mixture was cooled to room temperature and poured into a mixture of H₂O (2 L) and ice (1 kg). An aqueous solution of NaNO₂ (600 mL, 120 g NaNO₂, 20% by weight) was added followed by the slow addition of aqueous H₂SO₄ until the pH of the reaction mixture was 1. The precipitate was collected via filtration, washed with H₂O and dried in vacuo to afford 5-(4-iodo-1-methyl-1H-pyrazol-5-yl)-2H-tetrazole as a colorless solid (187 g, 90%).

### Alternative Method for Preparation 13:

To a solution of Preparation 12, 4-iodo-1-methyl-1H-pyrazole-5-carbonitrile (500 mg, 2.15 mmol) in 2-methyl tetrahydrofuran (4 mL) was added P₂S₅ (24 mg, 0.11 mmol) followed by hydrazine monohydrate (523 μL, 10.7 mmol). The reaction mixture was heated in a sealed vial at 70° C. for 17 h. The reaction mixture was added slowly to heptane with vigorous stirring until an oily precipitate formed. The mother liquor was decanted away and the residue triturated with heptane and dried under vacuum to afford a light yellow solid (520 mg). The residue was dissolved in EtOH (5 mL). HCl (2.0 mL, 3.0 M aqueous solution) was added followed by NaNO₂ (405 mg, 5.88 mmol) dissolved in H₂O (1.5 mL) dropwise to control exotherm and gas evolution. The reaction mixture was concentrated in vacuo to a volume of ˜3 mL. H₂O (20 mL) and DCM (15 mL) were added, followed by saturated aqueous NaHCO₃ (5 mL) to make the pH of the solution >7. The reaction mixture was partitioned and the organic layer discarded. The aqueous layer was acidified to pH 1 with 6M HCl. The reaction mixture was extracted with EtOAc (2×40 mL). The combined organic layers were dried with MgSO₄ and concentrated in vacuo to afford 5-(4-iodo-1-methyl-1H-pyrazol-5-yl)-2H-tetrazole as an off-white solid (390 mg, 66%).

¹H NMR (MeOH-d₄) δ: 7.69 (s, 1H), 4.08 (s, 3H).

UPLC (UPLC-MS Method 1): t_{R}=0.52 min.

MS (ES+): 276.9 (M+H)⁺.

### Preparation 14: ethyl 1-[5-(4-iodo-1-methyl-1H-pyrazol-5-yl)-2H-tetrazol-2-yl]ethyl carbonate

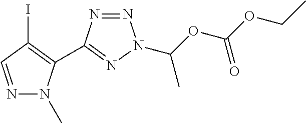 [SMILES: CCOC(=O)OC(C)n1nnc(-c2c(I)cnn2C)n1]

A round-bottom flask was charged with Preparation 13, 5-(4-iodo-1-methyl-1H-pyrazol-5-yl)-2H-tetrazole (191 g, 692 mmol), 4-dimethylaminopyridine (4.27 g, 34.6 mmol), THF (1.72 L), acetaldehyde (43 mL, 760 mmol), and triethylamine (107 mL, 762 mmol). The reaction solution was stirred and then ethyl chloroformate (86.2 mL, 97% by mass, 692 mmol) was added. The reaction mixture was stirred overnight at room temperature. The reaction mixture was diluted with EtOAc (965 mL) and H₂O (965 mL). The layers were separated. The aqueous layer was extracted with EtOAc (965 mL). The combined organic layers were dried over magnesium sulfate and concentrated in vacuo to afford ethyl 1-[5-(4-iodo-1-methyl-1H-pyrazol-5-yl)-2H-tetrazol-2-yl]ethyl carbonate as a colorless oil (261 g, 96% yield).

### Preparation 14a and 14b

### 14a: (S)-ethyl 1-[5-(4-iodo-1-methyl-1H-pyrazol-5-yl)-2H-tetrazol-2-yl]ethyl carbonate

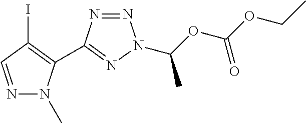 [SMILES: CCOC(=O)O[C@@H](C)n1nnc(-c2c(I)cnn2C)n1]

### 14b: (R)-ethyl 1-[5-(4-iodo-1-methyl-1H-pyrazol-5-yl)-2H-tetrazol-2-yl]ethyl carbonate

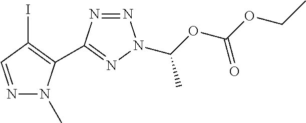 [SMILES: CCOC(=O)O[C@H](C)n1nnc(-c2c(I)cnn2C)n1]

407.5 g of Preparation 14, ethyl 1-[5-(4-iodo-1-methyl-1H-pyrazol-5-yl)-2H-tetrazol-2-yl]ethyl carbonate was processed according to Chiral Preparative Chromatography Method 1, followed by concentration of each enantiomer to dryness in vacuo to give isomer 14a (177.4 g, 99.22%, 99.79% e.e.; t_{R}=2.12 min) and isomer 14b (177.74 g, 98.83%, 98.46% e.e; t_{R}=2.59 min).

¹H NMR (MeOH-d₄) δ: 7.63 (s, 1H), 7.28 (q, 1H), 4.32-4.24 (m, 2H), 4.23 (s, 3H), 2.10 (d, 3), 1.33 (t, 3H).

UPLC (UPLC-MS Method 1): t_{R}=0.87 min.

MS (ES+): 393.0 (M+H)⁺.

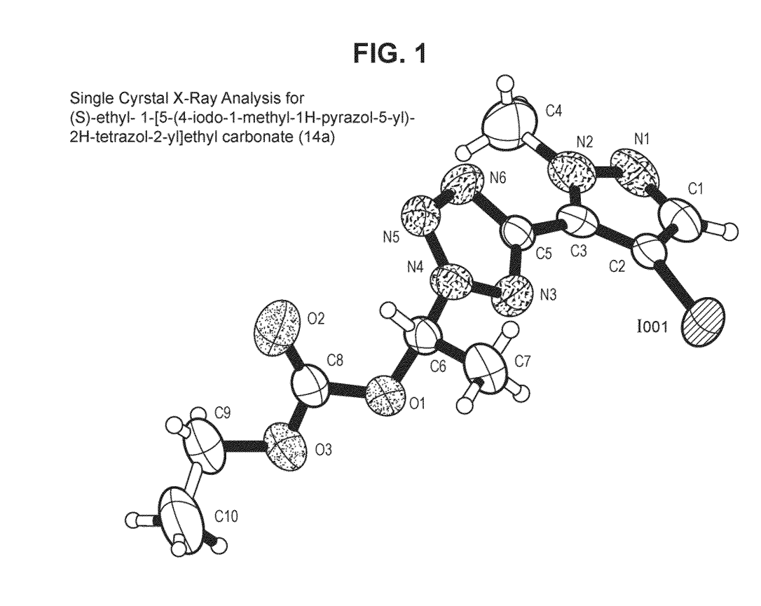

FIG. 1 is an ORTEP drawing of (S)-ethyl 1-[5-(4-iodo-1-methyl-1H-pyrazol-5-yl)-2H-tetrazol-2-yl]ethyl carbonate (14a).

Single Crystal X-Ray Analysis for (S)-ethyl 1-[5-(4-iodo-1-methyl-1H-pyrazol-5-yl)-2H-tetrazol-2-yl]ethyl carbonate (14a): Data collection was performed on a Bruker APEX diffractometer at room temperature. Data collection consisted of omega and phi scans. The structure was solved by direct methods using SHELX software suite in the space group P2₁. The structure was subsequently refined by the full-matrix least squares method. All non-hydrogen atoms were found and refined using anisotropic displacement parameters.

All hydrogen atoms were placed in calculated positions and were allowed to ride on their carrier atoms. The final refinement included isotropic displacement parameters for all hydrogen atoms. Absolute configuration was determined be examination of the Flack parameter. In this case, the parameter=0.0396 with an esd of 0.003. These values are within range for absolute configuration determination.

The final R-index was 3.5%. A final difference Fourier revealed no missing or misplaced electron density.

Pertinent crystal, data collection and refinement are summarized in Table 5.

TABLE 5
Crystal data and structure refinement for (S)-ethyl 1-[5-(4-
iodo-1-methyl-1H-pyrazol-5-yl)-2H-tetrazol-2-yl]ethyl carbonate.

| Empirical formula | C10 H13 I N6 O3 | |
|---|---|---|
| Formula weight | 392.16 | |
| Temperature | 293(2) K | |
| Wavelength | 1.54178 Å | |
| Crystal system | Monoclinic | |
| Space group | P2(1) | |
| Unit cell dimensions | a = 4.5885(4) Å | α = 90°. |
| | b = 10.0115(9) Å | β = 90.413(5)°. |
| | c = 16.2053(13) Å | γ = 90°. |

| | |
|---|---|
| Volume | 744.42(11) Å³ |
| Z | 2 |
| Density (calculated) | 1.750 Mg/m³ |
| Absorption coefficient | 17.076 mm⁻¹ |
| F(000) | 384 |
| Crystal size | 0.31 × 0.1 × 0.08 mm³ |
| Theta range for data collection | 5.19 to 70.22°. |
| Index ranges | −5 <= h <= 5, −12 <= k <= 11, −18 <= |
| | l <= 18 |
| Reflections collected | 12126 |
| Independent reflections | 2625 [R(int) = 0.0527] |
| Completeness to theta = 70.22° | 95.5% |
| Absorption correction | None |
| Refinement method | Full-matrix least-squares on F² |
| Data/restraints/parameters | 2625/1/184 |
| Goodness-of-fit on F² | 1.039 |
| Final R indices [I > 2sigma(I)] | R1 = 0.0355, wR2 = 0.0787 |
| R indices (all data) | R1 = 0.0511, wR2 = 0.0864 |
| Absolute structure parameter | 0.040(10) |
| Largest diff. peak and hole | 0.727 and −0.373 e.Å⁻³ |

### Preparation 15: ethyl 1-[5-(4-iodo-1-methyl-1H-pyrazol-5-yl)-1H-tetrazol-2-yl]ethyl carbonate

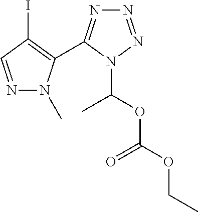 [SMILES: CCOC(=O)OC(C)n1nnnc1-c1c(I)cnn1C]

### Small Scale:

A round-bottom flask was charged with Preparation 13, 5-(4-iodo-1-methyl-1H-pyrazol-5-yl)-2H-tetrazole (790 mg, 2.86 mmol), DMF (15 mL), 1-chloroethyl ethylcarbonate (2.3 mL, 17 mmol), and diisopropylethylamine (5 mL, 29 mmol). The reaction was heated at 60° C. overnight, cooled and concentrated in vacuo. The residue was dissolved in EtOAc, washed 3×4% MgSO₄ solution then 1× brine. The organic layer was dried over magnesium sulfate and concentrated in vacuo. The residue was purified by MPLC with a 0-30% EtOAc/heptane gradient to afford ethyl 1-[5-(4-iodo-1-methyl-1H-pyrazol-5-yl)-1H-tetrazol-2-yl]ethyl carbonate as a white solid (135 mg, 12% yield).

### Alternative Method for Preparation 15:

A round-bottom flask was charged with Preparation 13, 5-(3-iodo-1-methyl-1H-pyrazol-5-yl)-1H-tetrazole (15.0 g, 54.3 mmol) and methyl tert-butyl ether (75 mL). Bis(tributyltin) oxide (16.2 g, 27.2 mmol) was added and the resulting mixture heated to reflux for 1 h, then cooled to room temperature and concentrated to a minimal volume. 1-Bromoethyl ethylcarbonate (18.0 g, 81.5 mmol) was charged in methyl tert-butyl ether (7.5 mL) and the reaction was allowed to stir at room temperature for 40 h. Upon completion, acetonitrile (105 mL) was added. The acetonitrile solution was washed with heptane (5×45 mL). The combined heptane layers were back extracted with acetonitrile (45 mL). The combined acetonitrile layers were then treated with potassium fluoride (3.16 g) in water (7.4 mL) and stirred at room temperature for 1 h. The resulting suspension was filtered and washed with methyl tert-butyl ether (75 mL). The organic layer was separated and concentrated to a minimal volume. Acetonitrile (75 mL) was added to precipitate a large amount of solids. The slurry was warmed until all solids dissolved, then allowed to cool slowly to room temperature and stirred overnight. The slurry was filtered and rinsed with acetonitrile to yield the white solid product (12.4 g, 58% yield) as a single regioisomer.

### Large Scale:

Preparation 13 (2.63 kg, 9.53 mol) and acetonitrile (7.9 L) were charged to a reactor. Triethylamine (1.59 L, 11.43 mol) and chloroethyl ethyl carbonate (1.53 L, 11.43 mol) were then added. The reactor contents were heated to reflux. After 5 h, the reactor contents were cooled and were filtered to remove solids. The filtrate which contains product was charged back into the reactor. The acetonitrile was removed and displaced with toluene.

The crude mixture, as a solution in toluene, was purified by chromatography (40-60μ SiO₂, 60×25 cm column) eluting with 95/5 toluene/acetonitrile to afford ethyl 1-[5-(4-iodo-1-methyl-1H-pyrazol-5-yl)-1H-tetrazol-2-yl]ethyl carbonate as a solid (920 g, 25% yield).

### Preparation 15a, 15b, and Derivative 15c

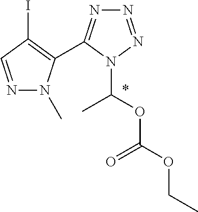 [SMILES: CCOC(=O)OC(C)n1nnnc1-c1c(I)cnn1C]

The small scale Preparation 15 (135 mg) was processed according to Chiral Preparative Chromatography Method 2, followed by concentration of each enantiomer to dryness in vacuo to give Preparation 15a (>99% e.e., t_{R}=4.80 min (Chiral Analytical Chromatography Method 1)) and Preparation 15b (90% e.e., t_{R}=5.28 min (Chiral Analytical Chromatography Method 1)).

The large scale Preparation 15 (907.2 g) was processed according to Chiral Preparative Chromatography Method 3, followed by concentration of each enantiomer to dryness in vacuo to give Preparation 15a (441.3 g, 99.6% e.e., t_{R}=4.80 min (Chiral Analytical Chromatography Method 1)) and Preparation 15b (435.6 g, 98.5% e.e., t_{R}=5.28 min (Chiral Analytical Chromatography Method 1)).

### Enzymatic Method for Preparation 15a:

To a jacketed 100 mL reactor (equipped with pH probe, overhead stirrer and burette) charged 42.5 mL of phosphate buffer (pH 7.5, 100 mM) and heated to 35° C. using water circulating bath. The reactor was then charged with 2.5 mL of liquid Candida Antarctica Lipase B enzyme solution, followed by 5 mL of substrate solution in acetonitrile (2.5 g of Prepartion 15 in 2.5 mL acetonitrile). The reaction was stirred at 35° C., while maintaining the reaction pH at 7.0, by titration with 1N NaOH solution. After 70 h, reaction was stopped and the gummy solids were allowed to settle and were collected by decanting off the liquid. The gummy solids were dissolved in ethanol and crystallized to provide Preparation 15a as a white solid (195 mg, 7.8%, >98% e.e.).

### Alternative Method for Preparation 15 and 15a:

### Step 1: 1-(1H-tetrazol-1-yl)ethyl ethyl carbonate

A 100 mL reactor was charged with tetrazole in acetonitrile (15.8 mL of 0.45 M solution, 7.14 mmol), acetaldehyde (0.80 mL, 14.3 mmol), 4-(dimethylamino)pyridine (45.0 mg, 0.357 mmol), and triethylamine (2.09 mL, 15.0 mmol). The reaction was cooled to 0° C. and ethyl chloroformate (1.37 mL, 14.3 mmol) was added via syringe pump, maintaining the reaction temperature below 5° C. The slurry was stirred for 1 h at 0° C., then warmed to 20° C. over 20 minutes and allowed to stir overnight. The reaction was quenched by addition of 10 mL water and 10 mL saturated NaCl solution and the organic layer was separated. The aqueous layer was extracted with EtOAc (10 mL). The combined organic layers were dried over MgSO₄, filtered and concentrated to produce an orange oil (6:1 ratio of regioisomeric products by proton NMR). The crude material was concentrated on silica gel and purified by column chromatography using 20-60% EtOAc/heptane as eluent to afford 1-(1H-tetrazol-1-yl)ethyl ethyl carbonate as an orange oil (0.964 g, 73% yield). Regiosiomeric assignment of the major product as the N1 regioisomer was confirmed by NOESY.

TLC: R_{f} of title compound (N1 regioisomer): 0.23 in 50% EtOAc/heptane; R_{f} of N2 regioisomer: 0.51 in 50% EtOAc/heptane

¹H NMR (CDCl₃) δ 8.87 (s, 1H), 6.90 (q, 1H), 4.29-4.19 (m, 2H), 2.07 (d, 3H), 1.32 (t, 3H).

### Step 2: 1-(5-bromo-1H-tetrazol-1-yl)ethyl ethyl carbonate

A 25 mL reaction vessel was charged with the compound from Step 1, 1-(1H-tetrazol-1-yl)ethyl ethyl carbonate (1.20 g, 6.45 mmol), 1,3-dibromo-5,5-dimethylhydantoin (2.10 g, 7.09 mmol) and acetic acid (12 mL) and placed under nitrogen. The reaction was warmed to 60° C. and stirred overnight. The reaction was cooled and poured over water (12 mL), then extracted with EtOAc (25 mL). The organic layer was washed with 10% NaHSO₃ (2×20 mL), followed by saturated NaHCO₃ (3×20 mL), then water (1×20 mL). The organic layer was dried over MgSO₄, filtered and concentrated, maintaining water bath below 30° C., to furnish 1-(5-bromo-1H-tetrazol-1-yl)ethyl ethyl carbonate as a clear oil (1.63 g, 95% yield).

¹H NMR (CDCl₃) δ 6.86 (q, 1H), 4.29-4.20 (m, 2H), 2.02 (d, 3H), 1.33 (t, 3H).

¹³C NMR (CDCl₃) δ 152.8, 133.0, 79.5, 65.5, 19.7, 14.0.

### Step 2a: (S)-1-(5-bromo-1H-tetrazol-1-yl)ethyl ethyl carbonate

To a jacketed 100 mL reactor (equipped with pH probe, overhead stirrer and burette) charged 50 mL of phosphate buffer (pH 7.0, 100 mM) and heated to 30° C. using a water circulating bath. The reactor was then charged with 1 mL of Candida Antarctica Lipase B enzyme solution, followed by 9 mL of substrate stock solution (prepared by dissolving 6.5 g of the compound from Step 2, 1-(5-bromo-1H-tetrazol-1-yl)ethyl ethyl carbonate, in 2.5 mL of acetonitrile). The reaction mixture stirred at 30° C., while maintain the reaction pH at 7.0 by titrating with 1N sodium hydroxide solution. After 6 h, reaction was stopped, transferred to a separating funnel and extracted with 70 mL of methyl tert butyl ether. The organic layer was collected, washed with water, dried over anhydrous sodium sulfate and concentrated under vacuum to give 2.75 g of liquid product (yield 42.3%, 97.5% e.e.).

### Step 3: ethyl (1-(5-(1-methyl-1H-pyrazol-5-yl)-1H-tetrazol-1-yl)ethyl)carbonate

A microwave vial was charged with the compound from Step 2, 1-(5-bromo-1H-tetrazol-1-yl)ethyl ethyl carbonate (300 mg, 1.13 mmol), 1-methyl-5-(tributylstannyl)-1H-pyrazole (504 mg, 1.36 mmol), dimethylformamide (5.7 mL), and tetrakis(triphenylphosphine)palladium(0) (65.4 mg, 0.0566 mmol). The vial was sealed with a septum cap and nitrogen gas was bubbled through the reaction mixture for 2 min. The reaction mixture was heated at 80° C. overnight. The reaction mixture was cooled, poured into H₂O (25 mL) and extracted with Et₂O (3×25 mL). The combined organic layers were dried over MgSO₄ and concentrated in vacuo. The residue was purified by silica gel chromatography, eluting with a 0-50% EtOAc/heptane gradient to afford ethyl (1-(5-(1-methyl-1H-pyrazol-5-yl)-1H-tetrazol-1-yl)ethyl)carbonate as a colorless solid (108 mg, 36% yield).

¹H NMR (CDCl₃) δ: 7.67 (d, 1H), 6.84 (q, 1H), 6.75 (d, 1H), 4.22-4.14 (m, 2H), 4.10 (s, 3H), 2.01 (d, 3H), 1.29 (t, 3H).

UPLC (UPLC-MS Method 1): t_{R}=0.73 min.

MS (ES+): 267.1 (M+H)⁺.

### Step 4: ethyl 1-[5-(4-iodo-1-methyl-1H-pyrazol-5-yl)-1H-tetrazol-2-yl]ethyl carbonate

A vial was charged with the compound from Step 3, ethyl (1-(5-(1-methyl-1H-pyrazol-5-yl)-1H-tetrazol-1-yl)ethyl)carbonate (103 mg, 0.387 mmol), MeCN (0.4 mL), iodine (49.1 mg, 0.193 mmol), iodic acid (13.6 mg, 0.0774 mmol), AcOH (0.1 mL), and H₂O (0.1 mL). The vial was sealed and the reaction mixture was heated at 50° C. overnight. The reaction mixture was cooled so that an additional portion of iodine (49.1 mg, 0.193 mmol) and iodic acid (13.6 mg, 0.0774 mmol) could be added, and then the reaction mixture was heated at 50° C. for 24 h. The reaction mixture was cooled and then diluted with EtOAc (20 mL). The organic layer was washed with aqueous Na₂SO₃ (20 mL) and brine (20 mL). The organic layer was dried over MgSO₄ and concentrated in vacuo to afford ethyl 1-[5-(4-iodo-1-methyl-1H-pyrazol-5-yl)-1H-tetrazol-2-yl]ethyl carbonate as a colorless solid (106 mg, 70% yield).

One skilled in the art will recognize that inclusion of Step 2a, followed by Steps 3 and 4 will allow for an alternative synthesis of Preparation 15a.

¹H NMR (CDCl₃) δ: 7.70 (s, 1H), 6.47 (q, 1H), 4.14-4.02 (m, 2H), 3.89 (s, 3H), 2.20 (d, 3), 1.24 (t, 3H).

UPLC (UPLC-MS Method 1): t_{R}=0.81 min.

MS (ES+): 393.3 (M+H)⁺.

The absolute configuration of the enantiomer 15a was determined by X-ray crystallography of a suitably derivatized molecule. Thus, a mixture of p-nitrophenyl boronic acid (300 mg, 1.8 mmol), Preparation 15a (705 mg, 1.8 mmol), Pd(dppf)₂Cl₂ (74 mg, 0.09 mmol) and CsF (1 N solution in water, 5.4 mL, 5.4 mmol) in dioxane (6 mL) was degassed by sparging with nitrogen for 10 min then sealed in a pressure bottle. The mixture was then heated at 95° C. After 2 h, the mixture was cooled, diluted with water (20 mL) and extracted with ethyl acetate (2×20 mL), dried over sodium sulfate, filtered and concentrated. The residue was purified by chromatography to provide product 15c.

¹H NMR (DMSO-d₆) δ: 8.26 (s, 1H), 8.22 (d, 2H), 7.27-7.32 (d, 2H), 6.31 (br. s., 1H), 3.84-4.03 (m, 2H), 3.81 (s, 3H), 1.43 (br. s., 3H), 1.07 (t, 3H)

UPLC (UPLC-MS Method 1): t_{R}=0.86 min.

MS (ES+): 388.3 (M+H)⁺.

A portion of the material was crystallized from ethyl acetate to give (S)-ethyl (1-(5-(1-methyl-4-(4-nitrophenyl)-1H-pyrazol-5-yl)-1H-tetrazol-1-yl)ethyl)carbonate.

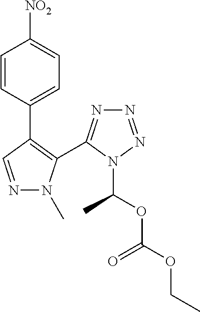 [SMILES: CCOC(=O)O[C@@H](C)n1nnnc1-c1c(-c2ccc([N+](=O)[O-])cc2)cnn1C]

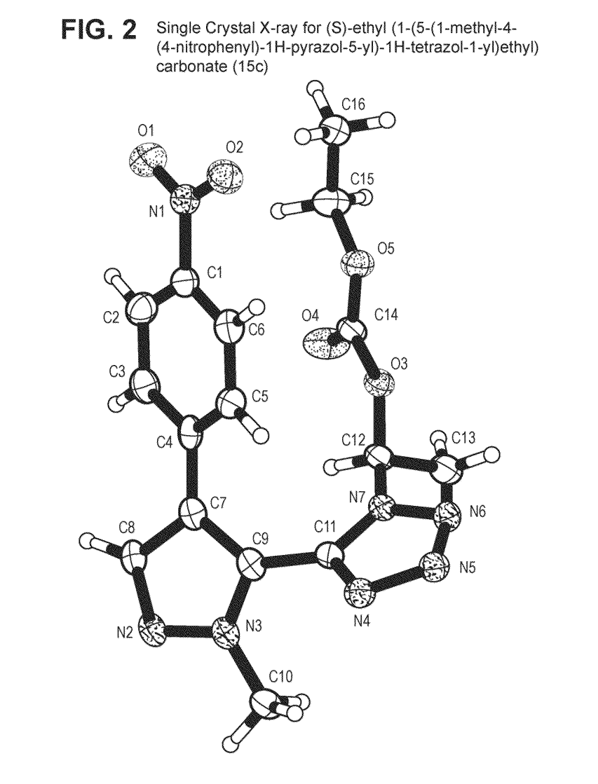

FIG. 2 is an ORTEP drawing of (S)-ethyl (1-(5-(1-methyl-4-(4-nitrophenyl)-1H-pyrazol-5-yl)-1H-tetrazol-1-yl)ethyl)carbonate (15c).

Single Crystal X-Ray Analysis for (S)-ethyl (1-(5-(1-methyl-4-(4-nitrophenyl)-1H-pyrazol-5-yl)-1H-tetrazol-1-yl)ethyl)carbonate (15c): Data collection was performed on a Bruker APEX diffractometer at a temperature of −150° C. Data collection consisted of omega and phi scans. The structure was solved by direct methods using SHELX software suite in the space group P2₁. The structure was subsequently refined by the full-matrix least squares method. All non-hydrogen atoms were found and refined using anisotropic displacement parameters. All hydrogen atoms were placed in calculated positions and were allowed to ride on their carrier atoms. The final refinement included isotropic displacement parameters for all hydrogen atoms. Analysis of the absolute structure using likelihood methods (Hooft 2008) was performed using PLATON (Spek 2010). The results indicate that the absolute structure has been correctly assigned. The method calculates that the probability that the structure is correct is 100.0. The Hooft parameter is reported as 0.01 with an esd of 0.012. The final R-index was 3.3%. A final difference Fourier revealed no missing or misplaced electron density.

Pertinent crystal, data collection and refinement for 15c are summarized in Table 6.

TABLE 6
Crystal data and structure refinement for (S)-ethyl (1-(5-(1-methyl-
4-(4-nitrophenyl)-1H-pyrazol-5-yl)-1H-tetrazol-1-yl)ethyl) carbonate.

| Empirical formula | C16 H17 N7 O5 | |
|---|---|---|
| Formula weight | 387.37 | |
| Temperature | 123(2) K | |
| Wavelength | 1.54178 Å | |
| Crystal system | Monoclinic | |
| Space group | P2(1) | |
| Unit cell dimensions | a = 9.1284(8) Å | α = 90°. |
| | b = 7.4486(7) Å | β = 107.149(6)°. |
| | c = 13.8629(11) Å | γ = 90°. |

| | |
|---|---|
| Volume | 900.68(14) Å³ |
| Z | 2 |
| Density (calculated) | 1.428 Mg/m³ |
| Absorption coefficient | 0.928 mm⁻¹ |
| F(000) | 404 |
| Crystal size | 0.50 × 0.16 × 0.10 mm³ |
| Theta range for data collection | 3.34 to 67.62°. |
| Index ranges | −10 <= h <= 10, −7 <= k <= 8, −16 <= |
| | l <= 16 |
| Reflections collected | 10283 |
| Independent reflections | 2827 [R(int) = 0.0382] |
| Completeness to theta = 67.62° | 97.2% |
| Absorption correction | Empirical |
| Max. and min. transmission | 0.9129 and 0.6540 |
| Refinement method | Full-matrix least-squares on F² |
| Data/restraints/parameters | 2827/1/256 |
| Goodness-of-fit on F² | 1.006 |
| Final R indices [I > 2sigma(I)] | R1 = 0.0333, wR2 = 0.0866 |
| R indices (all data) | R1 = 0.0347, wR2 = 0.0878 |
| Absolute structure parameter | 0.0(2) |
| Largest diff. peak and hole | 0.183 and −0.176 e.Å⁻³ |

### Example 1

### N-(3-methylpyridin-2-yl)-5-[1-methyl-5-(2H-tetrazol-5-yl)-1H-pyrazol-4-yl]-N-[(3R)-piperidin-3-yl]pyridine-2-carboxamide

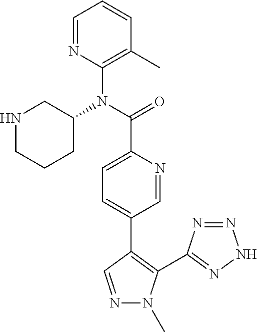 [SMILES: Cc1cccnc1N(C(=O)c1ccc(-c2cnn(C)c2-c2nn[nH]n2)cn1)[C@@H]1CCCNC1]

### Step 1:

Preparation 8, tert-butyl (R)-3-(5-bromo-N-(3-methylpyridin-2-yl)picolinamido)piperidine-1-carboxylate (1.85 g, 3.73 mmol), bis(pinacolato)diboron (1.42 g, 5.60 mmol), KOAc (1.10 g, 11.2 mmol) and PdCl₂(dppf) (76.2 mg, 0.0933 mmol) were dissolved in dioxane (10 mL). The reaction mixture was purged with N₂ and heated at 80° C. for 16 h. The reaction mixture was cooled and poured into water and extracted twice with ethyl acetate. The combined organic layers were washed with brine, dried over Na₂SO₄, and concentrated in vacuo. The crude material containing the desired aryl pinacol boronic ester and aryl boronic acid was used without further manipulation in the next reaction.

UPLC (UPLC-MS Method 1): t_{R}=0.78 min (boronic acid); 1.08 min (boronic ester).

MS (ES+): 440.2 (M+H)⁺ (boronic acid); 523.5 (M+H)⁺ (boronic ester).

### Step 2:

The crude product from Step 1 (282 mg, ˜0.640 mmol, based on aryl boronic acid), and Preparation 14a, (S)-ethyl 1-[5-(4-iodo-1-methyl-1H-pyrazol-5-yl)-2H-tetrazol-2-yl]ethyl carbonate (251 mg, 0.640 mmol), and PdCl₂(dppf) (26.1 mg, 0.0320 mmol) were dissolved in dioxane (5 mL) and aqueous 1 M CsF solution (1.92 mL, 1.92 mmol CsF). The reaction mixture was purged with N₂ and heated at 80° C. for 4 h. The reaction mixture was cooled and poured into sat NH₄Cl aqueous solution and extracted twice with ethyl acetate. The combined organic layers were washed with brine, dried over Na₂SO₄, and concentrated in vacuo. The residue was purified by silica gel column chromatography, eluting with a gradient of 25-80% EtOAc/heptane to afford the desired product (300 mg, 71%).

UPLC (UPLC-MS Method 1): t_{R}=1.00 min.

MS (ES+): 661.1 (M+H)⁺.

### Step 3:

The product of Step 2 (230 mg, 0.348 mmol) was dissolved in MeOH (2 mL). A solution of NaOH (145 mg, 3.64 mmol) in water (1 mL) was added and the reaction mixture was stirred at ambient temperature for 1 h. The pH reaction mixture was adjusted to 2 by the addition of aqueous 1N HCl and then extracted twice with ethyl acetate. The combined organic layers were washed with brine, dried over Na₂SO₄, and concentrated in vacuo. The crude material (180 mg, 95%) was used without further manipulation in the next reaction.

UPLC (UPLC-MS Method 1): t_{R}=0.80 min.

MS (ES+): 545.3 (M+H)⁺.

### Step 4:

The product of Step 3 (180 mg, 0.331 mmol) was dissolved in MeOH (1 mL). HCl (0.50 mL, 2.0 mmol, 4M solution in dioxane) was added. The reaction mixture was stirred at ambient temperature for 2 h. The reaction mixture was concentrated in vacuo to afford N-(3-methylpyridin-2-yl)-5-[1-methyl-5-(2H-tetrazol-5-yl)-1H-pyrazol-4-yl]-N-[(3R)-piperidin-3-yl]pyridine-2-carboxamide (146 mg, 92%).

¹H NMR (DMSO-d₆) δ: 9.37-9.00 (m, 2H), 8.97-8.61 (m, 1H), 8.27 (d, 1H), 8.20-8.10 (m, 1H), 8.06 (br s, 1H), 7.97 (s, 1H), 7.88-7.35 (m, 3H), 7.23 (m, 1H), 4.80-4.70 (m, 1H), 4.55-4.24 (m, 1H), 3.90 (s, 3H), 3.54-3.30 (m, 1H), 3.27-3.10 (m, 1H), 2.86-2.62 (m, 1H), 2.34-2.19 (m, 1H), 2.16-2.03 (m, 3H), 1.93-1.65 (m, 2H), 1.42-1.37 (m, 1H).

UPLC (UPLC-MS Method 1): t_{R}=0.48 min.

MS (ES+): 446.5 (M+H)⁺.

### Example 2

### N-(3-chloropyridin-2-yl)-5-[1-methyl-5-(2H-tetrazol-5-yl)-1H-pyrazol-4-yl]-N-[(3R)-piperidin-3-yl]pyridine-2-carboxamide

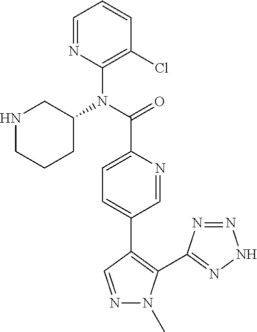 [SMILES: Cn1ncc(-c2ccc(C(=O)N(c3ncccc3Cl)[C@@H]3CCCNC3)nc2)c1-c1nn[nH]n1]

The title compound was made in an analogous manner to Example 1 starting from Preparation 7 and Preparation 14b.

¹H NMR (DMSO-d6) δ: 9.33 (br s, 1H), 8.95 (br s, 1H), 8.49 (s, 1H), 8.09-7.69 (m, 5H), 7.40 (dd, 1H), 4.93 (br s, 1H), 4.67-4.50 (m, 1H), 3.92 (s, 3H), 3.47-3.31 (m, 1H), 3.19 (d, 1H), 2.84-2.60 (m, 1H), 2.07-2.02 (m, 1H), 1.92-1.71 (m, 2H), 1.49-1.28 (m, 1H)

UPLC (UPLC-MS Method 1): t_{R}=0.50 min.

MS (ES+): 465.3 (M+H)⁺.

### Example 3

### N-(3-chloropyridin-2-yl)-3-fluoro-4-[1-methyl-5-(2H-tetrazol-5-yl)-1H-pyrazol-4-yl]-N-[(3R)-piperidin-3-yl]benzamide

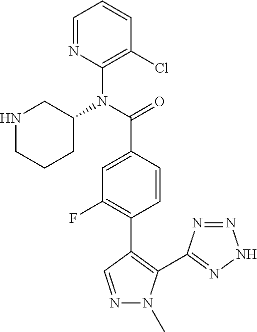 [SMILES: Cn1ncc(-c2ccc(C(=O)N(c3ncccc3Cl)[C@@H]3CCCNC3)cc2F)c1-c1nn[nH]n1]

The title compound was made in an analogous manner to Example 1 starting from Preparation 6 and Preparation 14a.

¹H NMR (DMSO-d6) δ: 8.91 (br s, 1H), 8.59 (br s, 1H), 7.96 (d, 1H), 7.81 (s, 1H), 7.47 (dd, 1H), 7.16 (dd, 1H), 7.03-6.99 (m, 2H), 4.96 (br s, 1H), 3.95 (s, 3H), 3.71-3.46 (m, 2H), 3.31-3.24 (m, 1H), 2.76-2.67 (m, 1H), 1.91-1.70 (m, 3H) 1.29-1.23 (m, 1H).

UPLC (UPLC-MS Method 1): t_{R}=0.53 min.

MS (ES+): 482.2 (M+H)⁺.

### Example 4

### N-(3-methylpyridin-2-yl)-3-fluoro-4-[1-methyl-5-(2H-tetrazol-5-yl)-1H-pyrazol-4-yl]-N-[(3R)-piperidin-3-yl]benzamide

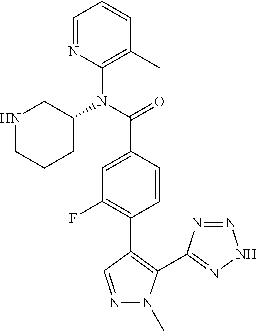 [SMILES: Cc1cccnc1N(C(=O)c1ccc(-c2cnn(C)c2-c2nn[nH]n2)c(F)c1)[C@@H]1CCCNC1]

The title compound was made in an analogous manner to Example 1 starting from Preparation 5 and Preparation 14b.

¹H NMR (DMSO-d6) δ: 8.43 (br s, 1H), 7.79 (s, 1H), 7.65 (d, 1H), 7.45 (s, 1H), 7.32 (s, 1H), 7.19 (s, 1H), 7.12 (dd, 1H), 6.94 (dd, 1H), 4.89 (br s, 1H), 3.95 (s, 3H), 3.55-3.46 (m, 1H), 3.40-3.33 (m, 1H), 3.18-3.15 (m, 1H), 2.14-2.09 (m, 1H), 2.02 (br s, 3H), 1.78 (br s, 3H), 1.26-1.22 (br s, 1H).

UPLC (UPLC-MS Method 1): t_{R}=0.50 min.

MS (ES+): 462.2 (M+H)⁺.

### Example 5a

### ethyl (S)-1-{5-[1-methyl-4-(4-{(3-methylpyridin-2-yl)[(3R)-piperidin-3-yl]carbamoyl}phenyl)-1H-pyrazol-5-yl]-1H-tetrazol-1-yl}ethyl carbonate

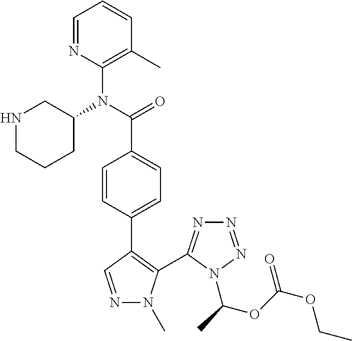 [SMILES: CCOC(=O)O[C@@H](C)n1nnnc1-c1c(-c2ccc(C(=O)N(c3ncccc3C)[C@@H]3CCCNC3)cc2)cnn1C]

The title compound 5a was made in an analogous manner to Example 1, Steps 2 and 4 starting from Preparation 10 and Preparation 15a.

¹H NMR (ACETONITRILE-d₃) δ: 9.71 (br s, 1H), 9.57-9.15 (m, 2H), 8.41 (br s, 1H), 8.07-7.87 (m, 2H), 7.81 (br s, 1H), 7.58-7.28 (m, 2H), 6.88 (br s, 2H), 5.97-5.87 (m, 1H), 5.05-4.06 (m, 1H), 4.04-3.95 (m, 2H), 3.80 (s, 3H), 3.62 (br s, 1H), 3.31 (d, 1H), 2.83 (br s, 1H), 2.30-2.12 (m, 3H), 2.05-1.83 (m, 4H), 1.52 (br s, 1H), 1.44 (t, 3H), 1.03 (br s, 3H).

UPLC (UPLC-MS Method 1): t_{R}=0.63 min.

MS (ES+): 560.3 (M+H)⁺.

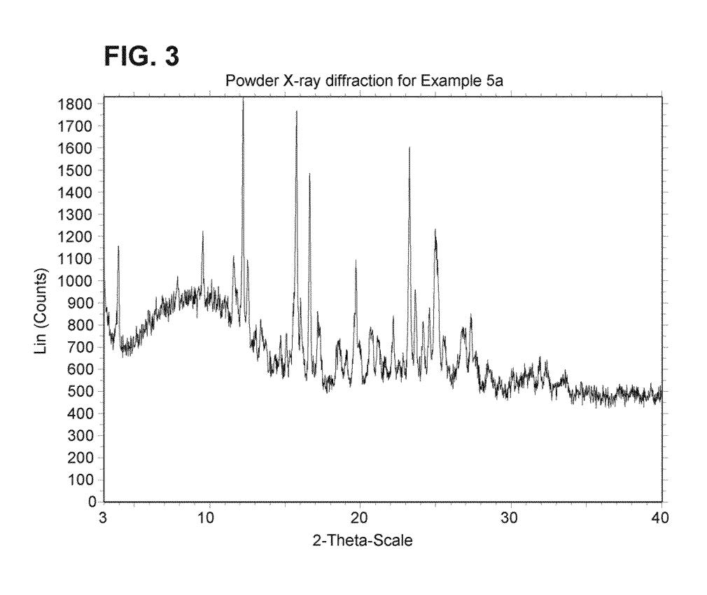

FIG. 3 shows the powder X-ray diffractogram.

### Example 5b

### ethyl (R)-1-{5-[1-methyl-4-(4-{(3-methylpyridin-2-yl)[(3R)-piperidin-3-yl]carbamoyl}phenyl)-1H-pyrazol-5-yl]-1H-tetrazol-1-yl}ethyl carbonate

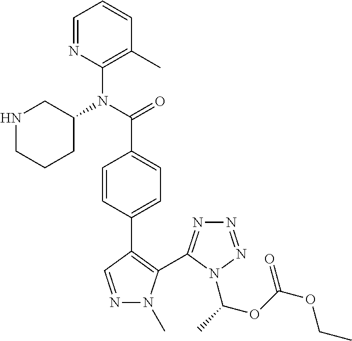 [SMILES: CCOC(=O)O[C@H](C)n1nnnc1-c1c(-c2ccc(C(=O)N(c3ncccc3C)[C@@H]3CCCNC3)cc2)cnn1C]

The title compound 5b was made in an analogous manner to Example 1, Steps 2 and 4 starting from Preparation 10 and Preparation 15b.

¹H NMR (ACETONITRILE-d3) δ: 8.39 (br s, 1H), 7.80 (s, 1H), 7.71 (br s, 1H), 7.41 (br s, 1H), 7.30 (br s, 2H), 6.86 (d, 2H), 5.92 (d, 1H), 5.02 (br s, 1H), 4.05-3.91 (m, 2H), 3.78 (s, 3H), 3.72-3.47 (m, 1H), 3.40-3.22 (m, 1H), 2.79 (br s, 1H), 2.25-2.10 (br m, 5H), 1.90-1.77 (m, 3H), 1.15-1.09 (m, 6H).

UPLC (UPLC-MS Method 2): t_{R}=0.63 min.

MS (ES+): 560.3 (M+H)⁺.

### Example 6

### ethyl (S)-1-{5-[1-methyl-4-(4-{(3-chloropyridin-2-yl)[(3R)-piperidin-3-yl]carbamoyl}phenyl)-1H-pyrazol-5-yl]-1H-tetrazol-1-yl}ethyl carbonate

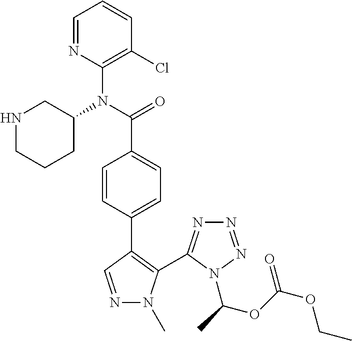 [SMILES: CCOC(=O)O[C@@H](C)n1nnnc1-c1c(-c2ccc(C(=O)N(c3ncccc3Cl)[C@@H]3CCCNC3)cc2)cnn1C]

The title compound was made in an analogous manner to Example 1, Steps 2 and 4 starting from Preparation 9 and Preparation 15a.

¹H NMR (ACETONITRILE-d₃) δ: 9.65-9.12 (br s, 1H), 8.50 (br s, 1H), 7.88-7.75 (m, 1H), 7.66 (d, 1H), 7.39-7.18 (m, 3H), 6.93-6.67 (m, 2H), 5.88 (q, 1H), 5.15-4.64 (m, 1H), 4.11-3.87 (m, 2H), 3.79 (s, 3H), 3.69-2.96 (m, 3H), 2.73-2.69 (m, 1H), 2.24-2.17 (m, 1H), 2.08-2.02 (m, 1H), 1.86-1.78 (m, 1H), 1.36-1.30 (m, 1H), 1.12 (t, 3H), 1.06 (d, 3H), 0.96 (br s, 1H).

UPLC (UPLC-MS Method 1): t_{R}=0.64 min.

MS (ES+): 580.3 (M+H)⁺.

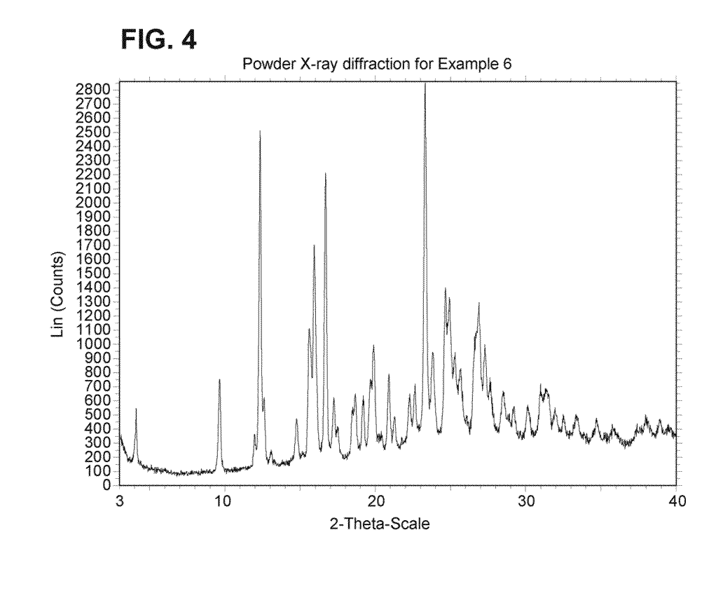

FIG. 4 shows the powder X-ray diffractogram for Example 6.

### Example 7

### ethyl (S)-1-{5-[4-(4-{(3-chloropyridin-2-yl)[(3R)-piperidin-3-yl]carbamoyl}-2-fluorophenyl)-1-methyl-1H-pyrazol-5-yl]-1H-tetrazol-1-yl}ethyl carbonate

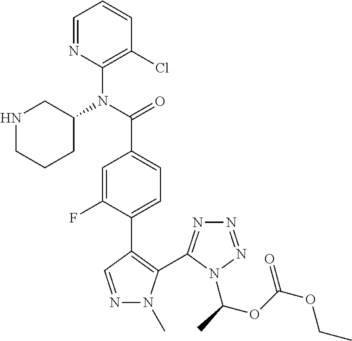 [SMILES: CCOC(=O)O[C@@H](C)n1nnnc1-c1c(-c2ccc(C(=O)N(c3ncccc3Cl)[C@@H]3CCCNC3)cc2F)cnn1C]

The title compound was made in an analogous manner to Example 1, Steps 1, 2, and 4 starting from Preparation 6 and Preparation 15a.

¹H NMR (ACETONITRILE-d3) δ: 8.55 (br s, 1H), 7.82 (br s, 1H), 7.74 (d, 1H), 7.36 (dd, 1H), 7.14 (d, 1H), 7.05 (d, 1H), 6.88 (dd, 1H), 5.93 (d, 1H), 5.19 (br s, 1H), 4.09-3.94 (m, 2H), 3.85 (s, 3H), 3.80-3.68 (m, 1H), 3.45 (br s, 1H), 3.33 (br s, 1H), 2.76 (br s, 1H), 2.04-1.85 (br m, 5H), 1.32 (br s, 2H), 1.16 (t, 3H).

UPLC (UPLC-MS Method 1): t_{R}=0.66 min.

MS (ES+): 598.2 (M+H)⁺.

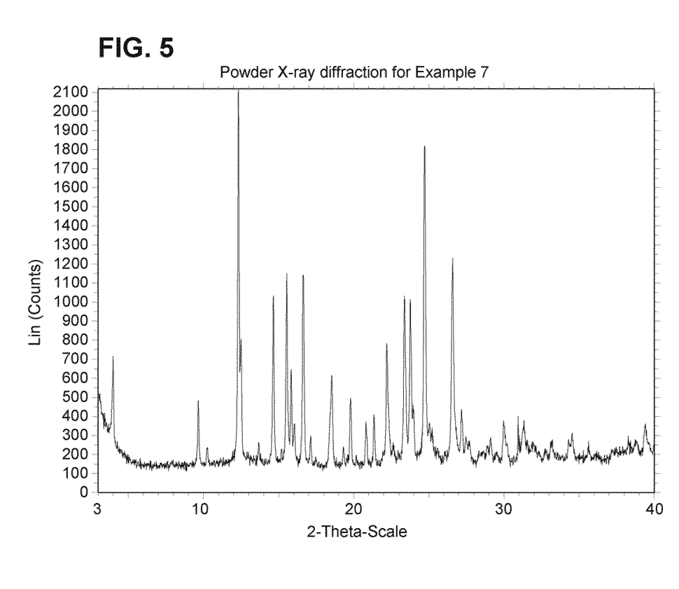

FIG. 5 shows the powder X-ray diffractogram for Example 7.

### Example 8

### ethyl (S)-1-{5-[4-(4-{(3-methylpyridin-2-yl)[(3R)-piperidin-3-yl]carbamoyl}-2-fluorophenyl)-1-methyl-1H-pyrazol-5-yl]-1H-tetrazol-1-yl}ethyl carbonate

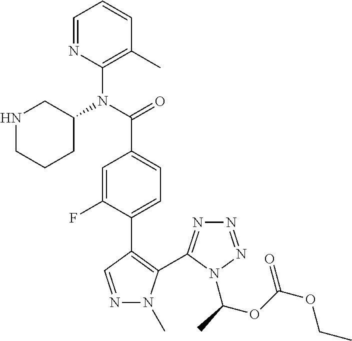 [SMILES: CCOC(=O)O[C@@H](C)n1nnnc1-c1c(-c2ccc(C(=O)N(c3ncccc3C)[C@@H]3CCCNC3)cc2F)cnn1C]

The title compound was made in an analogous manner to Example 1, Steps 1, 2, and 4 starting from Preparation 5 and Preparation 15a.

¹H NMR (ACETONITRILE-d3) δ: 8.42 (br s, 1H), 7.80 (s, 1H), 7.61-7.45 (m, 1H), 7.28 (br s, 1H), 7.13 (br s, 1H), 6.88 (br s, 1H), 5.92 (br s, 1H), 5.01-4.90 (m, 1H), 4.02-3.92 (m, 2H), 3.81 (s, 3H), 3.60 (br s, 1H), 3.29 (br s, 1H), 2.83 (br s, 1H), 2.22 (br s, 4H), 1.88-1.75 (m, 2H), 1.51 (br s, 1H), 1.12 (t, 3H), 1.06 (br s, 3H).

UPLC (UPLC-MS Method 1): t_{R}=0.63 min.

MS (ES+): 578.0 (M+H)⁺.

### Example 9

### ethyl (S)-1-{5-[1-methyl-4-(6-{(3-methylpyridin-2-yl)[(3R)-piperidin-3-yl]carbamoyl}pyridin-3-yl)-1H-pyrazol-5-yl]-1H-tetrazol-1-yl}ethyl carbonate

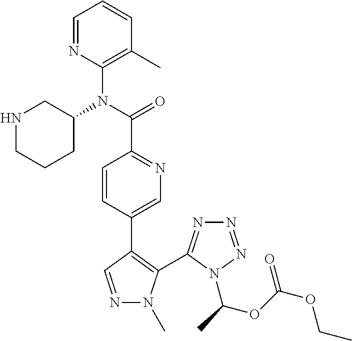 [SMILES: CCOC(=O)O[C@@H](C)n1nnnc1-c1c(-c2ccc(C(=O)N(c3ncccc3C)[C@@H]3CCCNC3)nc2)cnn1C]

The title compound was made in an analogous manner to Example 1, Steps 1, 2, and 4 starting from Preparation 8 and Preparation 15a.

¹H NMR (ACETONITRILE-d3) δ: 10.05-9.81 (br s, 1H), 9.68-9.28 (br m, 2H), 8.28 (br s, 1H), 8.06-7.85 (m, 2H), 7.85-7.69 (m, 2H), 7.55-7.30 (m, 2H), 6.02 (br s, 1H), 4.90-4.61 (m, 1H), 3.99 (q, 2H), 3.81 (s, 3H), 3.63 (br s, 1H), 3.34 (br s, 1H), 2.83 (br s, 1H), 2.45-2.14 (m, 4H), 1.88 (s, 3H), 1.79-1.58 (m, 1H), 1.34-1.19 (m, 3H), 1.15 (m, 3H).

UPLC (UPLC-MS Method 1): t_{R}=0.64 min.

MS (ES+): 561.3 (M+H)⁺.

### Example 10

### ethyl (S)-1-{5-[4-(6-{(3-chloropyridin-2-yl)[(3R)-piperidin-3-yl]carbamoyl}pyridin-3-yl)-1-methyl-1H-pyrazol-5-yl]-1H-tetrazol-1-yl}ethyl carbonate

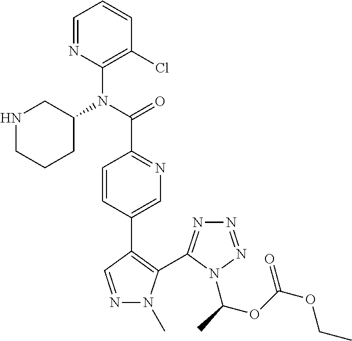 [SMILES: CCOC(=O)O[C@@H](C)n1nnnc1-c1c(-c2ccc(C(=O)N(c3ncccc3Cl)[C@@H]3CCCNC3)nc2)cnn1C]

The title compound was made in an analogous manner to Example 1, Steps 1, 2, and 4 starting from Preparation 7 and Preparation 15a.

¹H NMR (ACETONITRILE-d3) δ: 9.73-8.98 (br m, 2H), 8.45 (br s, 1H), 7.88 (br s, 2H), 7.81-7.65 (m, 2H), 7.48-7.23 (m, 2H), 6.04 (br s, 1H), 5.25-4.74 (m, 1H), 4.00 (q, 2H), 3.82 (s, 3H), 3.77-3.66 (m, 1H), 3.56 (d, 1H), 3.31 (d, 1H), 2.79 (br s, 1H), 2.26-2.09 (m, 1H), 1.91-1.82 (m, 2H), 1.56-1.41 (m, 1H), 1.26 (d, 3H), 1.15 (t, 3H).

UPLC (UPLC-MS Method 1): t_{R}=0.64 min.

MS (ES+): 581.2 (M+H)⁺.

### Example 11

### ethyl (R)-1-{5-[4-(4-{(3-chloropyridin-2-yl)[(3R)-piperidin-3-yl]carbamoyl}-2-fluorophenyl)-1-methyl-1H-pyrazol-5-yl]-1H-tetrazol-1-yl}ethyl carbonate

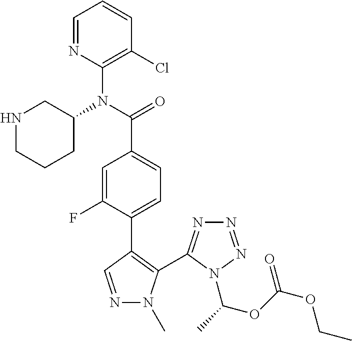 [SMILES: CCOC(=O)O[C@H](C)n1nnnc1-c1c(-c2ccc(C(=O)N(c3ncccc3Cl)[C@@H]3CCCNC3)cc2F)cnn1C]

The title compound was made in an analogous manner to Example 1, Steps 1, 2, and 4 starting from Preparation 6 and Preparation 15b.

¹H NMR (ACETONITRILE-d3) δ: 8.53 (br s, 1H), 7.83 (br s, 1H), 7.74 (d, 1H), 7.39 (dd, 1H), 7.15 (d, 1H), 7.05 (d, 1H), 6.88 (dd, 1H), 5.95 (d, 1H), 5.15 (br s, 1H), 4.03-3.94 (m, 2H), 3.85 (s, 3H), 3.75-3.63 (m, 1H), 3.40 (br s, 1H), 3.25 (br s, 1H), 2.75 (br s, 1H), 2.06-1.91 (m, 5H), 1.30 (br s, 2H), 1.15 (t, 3H)

UPLC (UPLC-MS Method 1): t_{R}=0.62 min.

MS (ES+): 598.4 (M+H)+.

### Example 12

### ethyl (R)-1-{5-[1-methyl-4-(4-{(3-chloropyridin-2-yl)[(3R)-piperidin-3-yl]carbamoyl}phenyl)-1H-pyrazol-5-yl]-1H-tetrazol-1-yl}ethyl carbonate

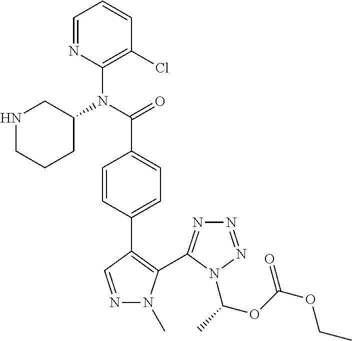 [SMILES: CCOC(=O)O[C@H](C)n1nnnc1-c1c(-c2ccc(C(=O)N(c3ncccc3Cl)[C@@H]3CCCNC3)cc2)cnn1C]

The title compound was made in an analogous manner to Example 1, Steps 2 and 4 starting from Preparation 9 and Preparation 15b.

¹H NMR (ACETONITRILE-d₃) δ: 9.45-9.06 (br d, 1H), 8.52 (d, 1H), 7.81 (s, 1H), 7.78-7.63 (m, 1H), 7.35-7.28 (m, 3H), 6.87 (d, 2H), 6.03-5.87 (m, 1H), 5.25-5.07 (m, 1H), 4.00 (q, 2H), 3.81 (s, 3H), 3.72-3.63 (m, 1H), 3.55 (br s, 1H), 3.50-3.35 (m, 1H), 3.24-3.33 (m, 1H), 2.62-2.84 (m, 1H), 2.10-2.18 (m, 4H), 1.30 (br s, 1H), 1.15 (t, 3H), 1.09 (br s, 1H)

UPLC (UPLC-MS Method 1): tR=0.72 min.

MS (ES+): 580.2 (M+H)+.

Throughout this application, various publications are referenced. The disclosures of these publications in their entireties are hereby incorporated by reference into this application for all purposes.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only.

Throughout this application, various publications are referenced. The disclosures of these publications in their entireties are hereby incorporated by reference into this application for all purposes.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only.

What is claimed is:

1.-18. (canceled)

19. The compound: ethyl (S)-1-{5-[4-(4-{(3-chloropyridin-2-yl)[(3R)-piperidin-3-yl]carbamoyl}-2-fluorophenyl)-1-methyl-1H-pyrazol-5-yl]-1H-tetrazol-1-yl}ethyl carbonate or a pharmaceutically acceptable salt thereof.

20. The compound

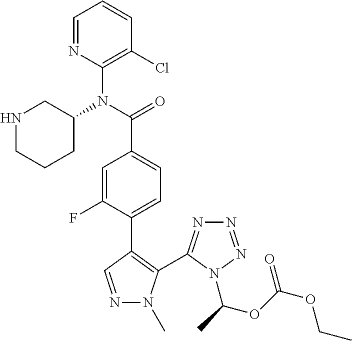 [SMILES: CCOC(=O)O[C@@H](C)n1nnnc1-c1c(-c2ccc(C(=O)N(c3ncccc3Cl)[C@@H]3CCCNC3)cc2F)cnn1C]

21.-40. (canceled)

41. A method for treating dyslipidemia, hypercholesterolemia, hypertriglyceridemia, hyperlipidemia, hypoalphalipoproteinemia, metabolic syndrome, diabetic complications, atherosclerosis, stroke, vascular dimensia, chronic kidney disease, coronary heart disease, coronary artery disease, retinopathy, inflammation, thrombosis, peripheral vascular disease or congestive heart failure in a mammal by administering to a mammal in need of such treatment a therapeutically effective amount of a compound of claim 19 or a pharmaceutically acceptable salt of said compound.

42. A pharmaceutical composition which comprises a therapeutically effective amount of a compound of claim 19 or a pharmaceutically acceptable salt of said compound and a pharmaceutically acceptable carrier, vehicle or diluent.

43. A pharmaceutical combination composition comprising: a therapeutically effective amount of a composition comprising
a first compound, said first compound being a compound of claim 19 or a pharmaceutically acceptable salt of said compound;
a second compound, said second compound being a lipase inhibitor, an HMG-CoA reductase inhibitor, an HMG-CoA synthase inhibitor, an HMG-CoA reductase gene expression inhibitor, an HMG-CoA synthase gene expression inhibitor, an MTP/Apo B secretion inhibitor, a CETP inhibitor, a bile acid absorption inhibitor, a cholesterol absorption inhibitor, a cholesterol synthesis inhibitor, a squalene synthetase inhibitor, a squalene epoxidase inhibitor, a squalene cyclase inhibitor, a combined squalene epoxidase/squalene cyclase inhibitor, a fibrate, niacin, a combination of niacin and lovastatin, an ion-exchange resin, an antioxidant, an ACAT inhibitor or a bile acid sequestrant, or a pharmaceutically acceptable salt of said compound; and
a pharmaceutically acceptable carrier, vehicle or diluents.

44. The compound: ethyl (S)-1-{5-[4-(4-{(3-chloropyridin-2-yl)[(3R)-piperidin-3-yl]carbamoyl}-2-fluorophenyl)-1-methyl-1H-pyrazol-5-yl]-1H-tetrazol-1-yl}ethyl carbonate hydrochloride.
